SEQUENCE LISTING (81 sequences)

SEQ ID NO: 1 - protein, 533 aa, Tripterygium wilfordii
MEFLLSLPTNTIAPKIFAVLLLFICLRILTNVLKPKKSKTSPPQASAAWPLIGHLLHLRGPQAPHITLGKMADKYGPIFKIKLGVHPTLVISDSEVAKECLTTHDIALAGRPATVAMEIMGYNHAMFAFSPYGPYWRHMRKLATVELLSAQRLETFKHIRESELKRSMKEMYQSWVHNKSGSGDSNHVTVDMTRILGDIIANVIYRMVVGKVYASKGEEDARWKQVVWEYIKLLSHFGVGDALPFLRWLDLGGVEKSMKKAAKELDIYVEEWLEEHKKKRSERKSDNGIVEEDFMDVMLSVFDDDDQLENFAHHSAHTINKAMCLAIILAASDTTKTTLTWALSLLLNHPDVMKKVQQELAAHIGPDKPVKESDVKSLVYLEAVVKETLRLYPPGPLGLPHESMEDCTVAGYHVPSGTRILYNLWKIQQDPQVWENPSEFKPDRFLTTHKDVDVRGRNFEYLPFGSGRRMCPGMSFALQVMEVSLANMLHGFDFATPNGKPVDMTEVNGLVTDRATPLEALITPRLPAHLYMG

SEQ ID NO: 2 - protein, 533 aa, Tripterygium wilfordii
MEFLLSLPTNTIAPKIFAVLLLFICLRILTNVLKPKKSKTSPPQASGAWPLIGHLLHLRGPQAPHITLGKMADKYGPIFKIKLGVHPTLVISDSEFAKECLTTHDIALAGRPATVAMEIMGYNHAMFAFSPYGPYCRHMRKLATVELLSAQRLETFKHIRESELKRSMKEMYQSWVHNKSGSGDSNHVTVDMTRILGDIIANVIYRMVVGKVYASKGEEDARWKQVVWEYIKLLSHFGVGDALPFLRWLDLGGVEKSMKKAAKELDIYVEEWLEEHKKKRSERKSDNGIVEEDFMDVMLSVFDDDDQLENFAHHSAHTINKAMCLAIILAASDTTKTTLTWALSLLLNHPDVMKKVQQELAAHIGPDKPVKESDVKSLVYLEAVVKETLRLYPPGPLGLPHESMEDCTVAGYHVPSGTRILYNLWKIQQDPQVWENPSEFKPDRFLTTHKDVDVRGRNFEYLPFGSGRRMCPGMSFALQVMEVSLANMLHGFDFATPNGKPVDMTEVNGLVTDRATPLEALITPRLPAHLYMG

SEQ ID NO: 3 - protein, 508 aa, Tripterygium wilfordii
MDLLQFPSVSILLGFVFFMFMVLKVWKRFEANGSTSNLPPGPWKLPIIGNLHQLGGSDPPHRALGELAKKYGPLMFLQLGEIQTLVVSSAEYAEEVLKTHDTVFASRPQMHSLEIMSYDYKDITFSPSDGSWRRRRKICVQELLSAKRVQSFRSTREKELSKLIQWIFSQAGTSINLTTKIYSSTCTLSSRMAFSDECKYQEEFISILKDLLKIASGFNIEDMFPSMKFLHLISGASSKIEKLHKQLDRIVGSIIDEHINLNTRKSEGNEDLVDVLLKYHEQGDSEFSLSMEEIKAIICDIYLAGTETSSTTVDWTMAELIKNPRVMKKAQAEVRQVFDSRGSVDETGIPELKYLKLVIKETLRLHPPGPLLLPRENAKSCEINEYVIPAKTRVMVNGWAIGRDPKYWPKEPEKFYPERFIDNPIDYKGTNFEYIPFGAGRRMCPGMAFGLANVELPLSQFLYYFDWKLADGMVPENLNMAEAFAATVCRKDDLYLIPTPYCPSPAFN

SEQ ID NO: 4 - protein, 499 aa, Tripterygium wilfordii
MDLQLPSFPILSSIILLILVVLKSVLRPSKLPPGPWKLPLIGNLHQLAQDLPHRALQKLAKKHGPLMHLHFGEVPTLVVTSPEYAKEVMKTHDITFASRPLLNAMKVMTYDHTDIAFAPYGEYWRQLRKICTIELLSVKRVQSFRPIREQETSNVIEWIGSNAGSSINLTERLYTTIYALVSKVAFGRTCGRGEHEEFIEYSKASQNRASGFNIVDVFPSLKLVHWIMGEGKKTERLHKQGDMLLGNIINQHVKKPVTGKGDDEHEDLVDVLLKFHEEGDFPLTINNIKSVIQDIFVAGGETSATTIDWAMREMMKNPRVMKKAQAEVRQVFDSRGRVDETAVPELKYLKLVLKETLRLHPPLPFLLPRINWERCEINGYEIAANTKVIVNAWAIGRDPNYWTEAERFYPERFLEKSADYKGTSFEYTPFGAGRRLCPGMSFGLANVEFPLSQLLYHFDWNLTGGMKPEDLNMIESFDVTMRAKDDLHLVPTPYRSLSG

SEQ ID NO: 5 - protein, 530 aa, Tripterygium wilfordii
MEFLLSLPTNTIATKIFAVLLLYLFLRIFTNVLKPKKSKTSPPQAGGAWPLIGHLHLLIGPQASYITLSKMADKYGPIFKIKLGVHPTLVISNSEVAKECLTTHDKVLANRPATVAMEIMGYNHAMFGWSPYGPYWRQLRKLVTVELLSNQRLKTFKHIRESEVKNSLKEMYQSWVHNKSGDSNHVSVDMTRIFGDITGNLIYRIVVGKVYARKGEGVVRWKQVVGDYMKLLTHFNVGDAMPFMRWFDLGGLEKAMKITFKELDGYVEEWLEEHKKKRSNSGGHGIVEEDFMDVMLSIFDDGGQQEYCTDNSTHTTNKAMCMALILGASETTKTTLTWSLSLLLNNLDVLKKVKQELAAHIGPETLVTESDVNSLVYLDAVITETLRLYPLGPLGLPHESIEDCTIAGYHVPARTRILFNLWKIHQDPRVWENPLEFKPERFLKEHNNIDVRGGHFELLPFGSGRRMCPGVSFALQVLKLTLANMLHGFDFATPNDEPVDMTEVNHMATTRATPLETLISPRLPSHLYMG

SEQ ID NO: 6 - protein, 533 aa, Tripterugium wilfordii
MEFLLSLPTNTIATTSFAVLLLYLCLRIFTNVLKPNKSKTSPPQAGGAWPLIGHLHLFRGPQPPHITLGKMADKHGPIFKIKLGVSPTLVISDSQIAKECFTTHDKILAGRPAYVALEIMGYNNAMFGFSPYGPYWRYIRKLATIELLSNKRLETFKHIRESEVKNAMKEMYQSWVVHNKSASGHSNHVSVDMSKILGDISSNVTYRAMVGKVYASKGEEDVRWKQVLSEYMKLLSNFSSCDALPFLRWFDFGGLEKSMKRTFKELDNYVEEWLQEHRKKRSSSGDGGIVVEDFMDVMLSIFDNVGEHENFTDYSPHTINKATCMSLLLGASDTTKSTMIWSLSLLLNHPDVLKKVQQELDAHIGPETLVNESDVKSFVYLDAVIKETLRLYSPGPLGLPHEAMEDCTVAGYHVPAGTQLLFNQWKMHQDPNVWEDPSEFKPERFLTTHKDIDFRGRHFEYLPFASGRRICPGISFAHQILMLSLANMLHGFDFTTPNGEPVDMAQVSGGTLIRATPLEALISPRLPGHVYMG

SEQ ID NO: 7 - protein, 530 aa, Tripterygium wilfordii
MEFLLSIPANTIATQIFALLLLYLCFRKFTDVLKPKQSKTSPPQVGGAWPLIGHLHRLRGPPAPHITLGKMADKYGPIFKIKLGLHPTLVISNSEIAKECLTTHDKVLAGRPATVATEIMSYNHAMFTFSSYGPYWSHTRKLVTVELLSNKRLETFKHIRESEVKNSVKEMYQSWVHNKTGDSNQVLVDMTRIFGDIIANVIYRIVVGKVYASKGEGHVRWKQVVSEYVNLLSHFGVGDALPFLRWLDLGGKEKAMKKTAKELDNYVEEWLQEHKKKRSSAGDHGIVEEDFMDVMLSIFYDDDQEESFADHSAHTINKALCLSLILAASDTTKTTLTWVLSLLLNHRDILNKVQQELIAHIGPETPVNESDIKSFVYLEAVIKETLRLYPPGPLGLPHESMEDCTIAGYHVPAGTRVLFNQWKIHHDPQVWENPSEFKPERFLRTHKEVDVRGRHFELLPFGSGRRMCPGISFALQVMELALANMLHGFDFATPNGEPVDMTEDNGFVTLRATPLEALISPRLPGHVYMG

SEQ ID NO: 8 - protein, 134 aa, Tripterygium wilfordii
MASDRKIYMFKEVETHNKTKDCWLIISGKVYDVTPFMEDHPGGDEVLLSSTGKDATNDFEDVGHSDNARDMMDQYCIGEIDGKTVPEKRNYIPAQTPAYNQDKTPEFVVKVLQFLVPLLILGLAFAVRHFTKKE

SEQ ID NO: 9 - protein, 708 aa, Tripterygium wilfordii
MQSSSNSMKASPLDLMSAIIKGKVDPSNVSSEVSGEVTSIIFENREFVMILTTSIAVLIGCVVVLIWRRSGAQKSKALVPPKPLAVKLPEPEVDDGKSKITVFYGTQTGTAEGFAKALVEEAKARYEKAVFKIVDLDDYAEDDDEYEEKLKKEKLAIFFLATYGDGEPTDNAARFYKWFLEGKERGECFQNMKFAVFGLGNRQYEHFNKVAKEVDQILSEQGATRLVPVGLGDDDQCLEDDFTAWRELVWPELDQLLRDKDGATTVSTPYTATIPEYRVKCYDTSDASVEEKSWSNANGHAVVDAQHPCRSNVAVKRELHTPASDRSCTHLEFDIAGTGLSYETGDHVGVYCENLTETVEEAVRLLGLSPDTYFSLHSDKEDGTPLSASSLPPTFPPCSLKTALARYADLLNSPKKSALLALAAHASDPTEADRLRHLASPAGKDEYAQWVIASQRSLLEIMAEFPSARPPLGVFFAAVAPRLQPRYYSISSSPRMAPSRIHVTCALVYEKTPAGRIHKGVCSTWMKNAVPLEKSHESCWAPIFVRQSNFKLPVDTKVPIIMIGPGTGFAPFRGFLQERLALKESGAELGSSILFFGCRNRRLDYIYEEELNNFVESAALSELIVAFSREGPTKEYVQHKMMEKASDIWNMINQGAYIYVCGDAKGMARDVHRTLHTIVQEQGSLDSSKAESMVKNLQTSGRYLRDVW

SEQ ID NO: 10 - DNA, 38 bp, Artificial
  Primer CO_TwCYP71BE85v1_TEF-F
  misc_feature (10)..(10): A, C, G or T
agcgatacgnaaaatggacttattgcaatttccatctg

SEQ ID NO: 11 - DNA, 29 bp, Artificial
  Primer CO_TwCYP71BE85v1_TEF-R
  misc_feature (8)..(8): A, C, G or T
cacgcgantcagttaaatgcgggtgatgg

SEQ ID NO: 12 - DNA, 34 bp, Artificial
  Primer CO_TwGA3OX1_TEF-F
  misc_feature (10)..(10): A, C, G or T
agcgatacgnaaaatgagtcctccgcctacaata

SEQ ID NO: 13 - DNA, 32 bp, Artificial
  Primer CO_TwGA3OX1_TEF-R
  misc_feature (8)..(8): A, C, G or T
cacgcganttaaatacctaaaagcgagacggg

SEQ ID NO: 14 - DNA, 38 bp, Artificial
  Primer CO_AcoUGT2_TEF-F
  misc_feature (10)..(10): A, C, G or T
agcgatacgnaaaatggctgttagcttaaaaaataccg

SEQ ID NO: 15 - DNA, 30 bp, Artificial
  Primer CO_AcoUGT2_TEF-R
  misc_feature (8)..(8): A, C, G or T
cacgcganttaacgactgatatgagcgacg

SEQ ID NO: 16 - DNA, 35 bp, Artificial
  Primer CO_TwCYP82D213_PGK-F
  misc_feature (10)..(10): A, C, G or T
atcaacgggnaaaatggaattccttctgtcattgc

SEQ ID NO: 17 - DNA, 32 bp, Artificial
  Primer CO_TwCYP82D213_PGK-R
  misc_feature (8)..(8): A, C, G or T
cgtgcganctaacccatgtaaagatgtgatgg

SEQ ID NO: 18 - DNA, 38 bp, Artificial
  Primer CO_TwCYP71BE86_PGK-F
  misc_feature (10)..(10): A, C, G or T
atcaacgggnaaaatggacttacaattacctagcttcc

SEQ ID NO: 19 - DNA, 33 bp, Artificial
  Primer CO_TwCYP71BE86_PGK-R
  misc_feature (8)..(8): A, C, G or T
cgtgcganttaaccagataaactacgatatggg

SEQ ID NO: 20 - DNA, 40 bp, Artificial
  Primer TwCYP82D217_pLife-F
  misc_feature (8)..(8): A, C, G or T
ggcttaanaagcatcttctctcctaactagctttctaaat

SEQ ID NO: 21 - DNA, 35 bp, Artificial
  Primer TwCYP82D217_pLife-R
  misc_feature (8)..(8): A, C, G or T
ggtttaanctattgcaattcaccccatgtagacaa

SEQ ID NO: 22 - DNA, 29 bp, Artificial
  Primer pLifeUP_TEF-F
  misc_feature (10)..(10): A, C, G or T
agcgatacgngacctgcaggctgaggctt

SEQ ID NO: 23 - DNA, 27 bp, Artificial
  Primer pLife_TEF-R
  misc_feature (8)..(8): A, C, G or T
cacgcgancccggggctgaggtttaat

SEQ ID NO: 24 - DNA, 35 bp, Artificial
  Primer TwCYP82D274v1_pLife-F
  misc_feature (8)..(8): A, C, G, or T
ggcttaanatggagtttcttctttcactcccaaca

SEQ ID NO: 25 - DNA, 35 bp, Artificial
  Primer TwCYP82D274v1_pLife-R
  misc_feature (8)..(8): A, C, G or T
ggtttaantcagcccatatagagatgagctgggag

SEQ ID NO: 26 - DNA, 31 bp, Artificial
  Primer pLife_TEF-F
  misc_feature (10)..(10): A, C, G or T
agcgatacgntgcaggctgaggcttaatatg

SEQ ID NO: 27 - DNA, 27 bp, Artificial
  Primer pLife_TEF-R
  misc_feature (8)..(8): A, C, G or T
cacgcgancccggggctgaggtttaat

SEQ ID NO: 28 - DNA, 36 bp, Artificial
  Primer TwCPR1_pLife-F
  misc_feature (8)..(8): A,. C, G or T
ggcttaanatgcaatcttcttcaaattctatgaagg

SEQ ID NO: 29 - DNA, 29 bp, Artificial
  Primer TwCPR1_pLife-R
  misc_feature (8)..(8): A, C, G or T
ggtttaanttaccacacatcccggagata

SEQ ID NO: 30 - DNA, 31 bp, Artificial
  Primer pLife_PGK-F
  misc_feature (10)..(10): A, C, G or T
atcaacgggntgcaggctgaggcttaatatg

SEQ ID NO: 31 - DNA, 27 bp, Artificial
  Primer pLife_PGK-R
  misc_feature (8)..(8): A, C, G or T
cgtgcgancccggggctgaggtttaat

SEQ ID NO: 32 - DNA, 29 bp, Artificial
  Primer Twb5#1_pLife-F
  misc_feature (8)..(8): A, C, G or T
ggcttaanatggcttcggatcggaagata

SEQ ID NO: 33 - DNA, 34 bp, Artificial
  Primer Twb5#1_pLife-R
  misc_feature (8)..(8): A, C, G or T
ggtttaanctattctttcttggtgaagtgacgta

SEQ ID NO: 34 - DNA, 31 bp, Artificial; identical to SEQ ID NO: 30
  Primer pLife_PGK-F
  misc_feature (10)..(10): A, C, G or T

SEQ ID NO: 35 - DNA, 27 bp, Artificial
  Primer pLife_PGK-R
  misc_feature (8)..(8): A, C, G or T
cgtgcgancccggggctgaggtttaat

SEQ ID NO: 36 - DNA, 29 bp, Artificial
  Primer Twb5#2_pLife-F
  misc_feature (8)..(8): A, C, G or T
ggcttaanatgggtggagacggaaaggtt

SEQ ID NO: 37 - DNA, 32 bp, Artificial
  Primer Twb5#2_pLife-R
  misc_feature (8)..(8): A, C, G or T
ggtttaanttaagcaggaggagctgatttggt

SEQ ID NO: 38 - DNA, 31 bp, Artificial; identical to SEQ ID NO: 30
  Primer pLife_PGK-F
  misc_feature (10)..(10): A, C, G or T

SEQ ID NO: 39 - DNA, 27 bp, Artificial
  Primer pLife_PGK-R
  misc_feature (8)..(8): A, C, G or T
cgtgcgancccggggctgaggtttaat

SEQ ID NO: 40 - DNA, 31 bp, Artificial
  Primer Twb5#3_pLife-F
  misc_feature (8)..(8): A, C, G or T
ggcttaanatggctggtcagagagttttcac

SEQ ID NO: 41 - DNA, 33 bp, Artificial
  Primer Twb5#3_pLife-R
  misc_feature (8)..(8): A, C, G, or T
ggtttaanttagaagatctgctcaggccttgta

SEQ ID NO: 42 - DNA, 31 bp, Artificial; identical to SEQ ID NO: 30
  Primer pLife_PGK-F
  misc_feature (10)..(10): A, C, G or T

SEQ ID NO: 43 - DNA, 27 bp, Artificial
  Primer pLife_PGK-R
  misc_feature (8)..(8): A, C, G or T
cgtgcgancccggggctgaggtttaat

SEQ ID NO: 44 - DNA, 40 bp, Artificial
  Primer Twb5#4_PGK-F
  misc_feature (10)..(10): A, C, G or T
atcaacgggnaaaatggctaaacttctttcatttgctgag

SEQ ID NO: 45 - DNA, 36 bp, Artificial
  Primer Twb5#4_PGK-R
  misc_feature (8)..(8): A, C, G or T
cgtgcganttagaaaaggtatcgcaaaccaaatgcc

SEQ ID NO: 46 - DNA, 38 bp, Artificial
  Primer Twb5#5_PGK-F
  misc_feature (10)..(10): A, C, G or T
atcaacgggnaaaatgattattgttgcggtggctctga

SEQ ID NO: 47 - DNA, 42 bp, Artificial
  Primer Twb5#5_PGK-R
  misc_feature (8)..(8): A, C, G or T
cgtgcganttacttctctagatccccaatgtaaaaatcatcg

SEQ ID NO: 48 - DNA, 37 bp, Artificial
  Primer Twb5#6_PGK-F
  misc_feature (10)..(10): A, C, G or T
  misc_feature (10)..(10): n is a, c, g, or t
atcaacgggnaaaatgccgactttaacgaagctgcac

SEQ ID NO: 49 - DNA, 34 bp, Artificial
  Primer Twb5#6_PGK-R
  misc_feature (8)..(8): A, C, G or T
  misc_feature (8)..(8): n is a, c, g, or t
cgtgcganctacttcttccgcaagtacaggagtc

SEQ ID NO: 50 - DNA, 27 bp, artificial
  Primer YEA85_UP_Genotyping_Fw
tctcaggtatagcatgaggtcgctcat

SEQ ID NO: 51 - DNA, 28 bp, artificial
  primer YEA86_DW_Genotyping_Fw
cctgcaggactagtgctgaggcattaat

SEQ ID NO: 52 - DNA, 20 bp, artificial
  primer YEA87_X-2_Genotyping_UP
gtttgtagttggcggtggag

SEQ ID NO: 53 - DNA, 20 bp, artificial
  primer YEA88_X-2_Genotyping_DW
gagacaagatggggcaagac

SEQ ID NO: 54 - DNA, 20 bp, artificial
  primer YEA89_X-3_Genotyping_UP
tgacgaatcgttaggcacag

SEQ ID NO: 55 - DNA, 21 bp, artificial
  primer YEA90_X-3_Genotyping_DW
ccgtgcaataccaaaatcgag

SEQ ID NO: 56 - DNA, 20 bp, artificial
  primer YEA91_X-4_Genotyping_UP
ctcacaaagggacgaatcct

SEQ ID NO: 57 - DNA, 19 bp, artificial
  primer YEA92_X-4_Genotyping_DW
gacggtacgttgaccagag

SEQ ID NO: 58 - DNA, 23 bp, artificial
  primer YEA93_XI-1_Genotyping_UP
cttaatgggtagtgcttgacacg

SEQ ID NO: 59 - DNA, 20 bp, artificial
  primer YEA94_XI-2_Genotyping_UP
gtttgtagttggcggtggag

SEQ ID NO: 60 - DNA, 20 bp, artificial
  primer YEA95_X1-2_Genotyping_DW
gagacaagatggggcaagac

SEQ ID NO: 61 - DNA, 25 bp, artificial
  primer YEA96_XI-5_Genotyping_UP
ctcaatgatcaaaatcctgaatgca

SEQ ID NO: 62 - DNA, 20 bp, artificial
  primer YEA97_X1-5_Genotyping_DW
gcatggtcaccgctatcagc

SEQ ID NO: 63 - DNA, 19 bp, artificial
  primer YEA98_XII-2_Genotyping_UP
cgaagaaggcctgcaattc

SEQ ID NO: 64 - DNA, 19 bp, Artificial
  primer YEA99_X1I-2_Genotyping_DW
ggccctgataaggttgttg

SEQ ID NO: 65 - DNA, 20 bp, Artificial
  Primer YEA100_XII-5_Genotyping_UP
ccaccgaagttgatttgctt

SEQ ID NO: 66 - DNA, 20 bp, Artificial
  Primer YEA101_X1I-5_Genotyping_DW
gtgggagtaagggatcctgt

SEQ ID NO: 67 - protein, 786 aa, Plectranthus barbatus
MGSLSTMNLNHSPMSYSGILPSSSAKAKLLLPGCFSISAWMNNGKNLNCQLTHKKISKVAEIRVATVNAPPVHDQDDSTENQCHDAVNNIEDPIEYIRTLLRTTGDGRISVSPYDTAWVALIKDLQGRDAPEFPSSLEWIIQNQLADGSWGDAKFFCVYDRLVNTIACVVALRSWDVHAEKVERGVRYINENVEKLRDGNEEHMTCGFEVVFPALLQRAKSLGIQDLPYDAPVIQEIYHSREQKSKRIPLEMMHKVPTSLLFSLEGLENLEWDKLLKLQSADGSFLTSPSSTAFAFMQTRDPKCYQFIKNTIQTFNGGAPHTYPVDVFGRLWAIDRLQRLGISRFFESEIADCIAHIHRFWTEKGVFSGRESEFCDIDDTSMGVRLMRMHGYDVDPNVLKNFKKDDKFSCYGGQMIESPSPIYNLYRASQLRFPGEQILEDANKFAYDFLQEKLAHNQILDKWVISKHLPDEIKLGLEMPWYATLPRVEARYYIQYYAGSGDVWIGKTLYRMPEISNDTYHELAKTDFKRCQAQHQFEWIYMQEWYESCNMEEFGISRKELLVAYFLATASIFELERANERIAWAKSQIISTIIASFFNNQNTSPEDKLAFLTDFKNGNSTNMALVTLTQFLEGFDRYTSHQLKNAWSVWLRKLQQGEGNGGADAELLVNTLNICAGHIAFREEILAHNDYKTLSNLTSKICRQLSQIQNEKELETEGQKTSIKNKELEEDMQRLVKLVLEKSRVGINRDMKKTFLAVVKTYYYKAYHSAQAIDNHMFKVLFEPVA

SEQ ID NO: 68 - protein, 598 aa, Plectranthus barbatus
MSSLAGNLRVIPFSGNRVQTRTGILPVHQTPMITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 69 - protein, 807 aa, Tripterygium wilfordii
MHSLLMKKVIMYSSQTTHVFPSPLHCTIPKSSSFFLDAPVARLHCLSGHGAKKKRLHFDIQQGRNAVSKTHTPDDLYAKQEYSVPEIVKDDDKEEEVVKIKEHVDIIKSMLSSMEDGEISISAYDTAWVALIQDIHNNGAPQFPSSLLWIAENQLPDGSWGDSRVFLAFDRIINTLACVVALKSWNVHPDKCERGISFLKENISMLEKDDSEHMLVGFEFGFPVLLDMARRLGIDVPDDSPFLQEIYVQRDLKLKRIPKDILHNVPTTLLHSLEAIPDLDWTKLLKLQCQDGSLLFSPSSTAMAFINTKDENCLRYLNYVVQRFNGGAPTVYPYDLFEHNWAVDRLQRLGISRFFQPEIRECMSYVYRYWTKDGIFCTRNSRVHDVDDTAMGFRLLRLHGYEVHPDAFRQFKKGCEFICYEGQSHPTVTVMYNLYRASQLMFPEEKILDEAKQFTEKFLGEKRSANKLLDKWIITKDLPGEVGFALDVPWYASLPRVEARFFIQHYGGEDDVWLDKALYRMPYVNNNVYLELAKLDYNYCQALHRTEWGRIQKWYEECKPRDFGISRECLLRAYFMAAASIFEPERSMERLAWAKTAILLEIIVSYFSEVGNSTEQRIAFTTEFSIRASPMGGYINGRKLDKIGTTQELIQMLLATIDQFSQDAFAAYGHDITRHLHNSWKMWLLKWQEEGDRWLGEAELLIQTINLMADHKIAEKLFMGHTNYEQLFSLTNKVCYSLGHHELQNNRELEHDMQRLVQLVLTNSSDGIDSDIKKTFLAVAKRFYYTAFVDPETVNVHIAKVLFERVD

SEQ ID NO: 70 - protein, 589 aa, Tripterygium wilfordii
MAPLVVSLTISHFVIQTGSTALHYSALPETRTKHCHSSRPFASINSNSLQMNQRPLTDYRPAIWNPELIDSLNTPYSYQSHGTQLDKLRQDAKRLLSSTSDPCLLLNHVESMQRLGIAYHFQEEIDYLLNTRIQPYSPDDHDLHTTALRFRILRDNNFPISSDVFGKFMSREGKFLDSLSRDVKGLLSLYEASFLGVDGEVILDEAKEFSSKNLRALLGRLESTSIDVAEQVKQSLQIPLFWRMPRVEARNFIDFYQKKDAKSSTLLELAKLDFNLVQSTYQQELKELSKWWENLGFKQKLSFTRDRLMQSYFSTTGITFKPQFSKARIAATKFINIVNTIDDIHDYYGSQDDLKLFDSAVKRWDLAAMEELPDYMKICYFAMYNLVNELAYDVLINQGIDVLPCLREAWTKFCGAAFVESQWCYTGYTPSMDDYLKNCWISIGVHGSLNFARAHQQGSRSPIANTPLHCLEDPLLYWSSVICRLNNDLATFQHESKTGEVVSFVKCYMVEKGVSQEQACDEIRELIKHAWKMLNTERRRSDLPPLMVEMCMDTPKLSQCLYQHGDGFGVAIDLTKDVMSSLIFRQIPI

SEQ ID NO: 71 - protein, 793 aa, Salvia miltiorrhiza
MASLSSTILSRSPAARRRITPASAKLHRPECFATSAWMGSSSKNLSLSYQLNHKKISVATVDAPQVHDHDGTTVHQGHDAVKNIEDPIEYIRTLLRTTGDGRISVSPYDTAWVAMIKDVEGRDGPQFPSSLEWIVQNQLEDGSWGDQKLFCVYDRLVNTIACVVALRSWNVHAHKVKRGVTYIKENVDKLMEGNEEHMTCGFEVVFPALLQKAKSLGIEDLPYDSPAVQEVYHVREQKLKRIPLEIMHKIPTSLLFSLEGLENLDWDKLLKLQSADGSFLTSPSSTAFAFMQTKDEKCYQFIKNTIDTFNGGAPHTYPVDVFGRLWAIDRLQRLGISRFFEPEIADCLSHIHKFWTDKGVFSGRESEFCDIDDTSMGMRLMRMHGYDVDPNVLRNFKQKDGKFSCYGGQMIESPSPIYNLYRASQLRFPGEEILEDAKRFAYDFLKEKLANNQILDKWVISKHLPDEIKLGLEMPWLATLPRVEAKYYIQYYAGSGDVWIGKTLYRMPEISNDTYHDLAKTDFKRCQAKHQFEWLYMQEWYESCGIEEFGISRKDLLLSYFLATASIFELERTNERIAWAKSQIIAKMITSFFNKETTSEEDKRALLNELGNINGLNDTNGAGREGGAGSIALATLTQFLEGFDRYTRHQLKNAWSVWLTQLQHGEADDAELLTNTLNICAGHIAFREEILAHNEYKALSNLTSKICRQLSFIQSEKEMGVEGEIAAKSSIKNKELEEDMQMLVKLVLEKYGGIDRNIKKAFLAVAKTYYYRAYHAADTIDTHMFKVLFEPVA

SEQ ID NO: 72 - protein, 595 aa, Salvia miltiorrhiza
  misc_feature (221)..(221): Xaa can be any naturally occurring amino acid
MSLAFNPAATAFSGNGARSRRENFPVKHVTVRGFPMITNKSSFAVKCNLTTTDLMGKIAEKFKGEDSNFPAAAAVQPAADMPSNLCIIDTLQRLGVDRYFRSEIDTILEDTYRLWQRKERAIFSDTAIHAMAFRLLRVKGYEVSSEELAPYADQEHVDLQTIEVATVIELYRAAQERTGEDESSLKKLHAWTTTFLKQKLLTNSIPDKKLHKLVEYYLKNXHGILDRMGVRQNLDLYDISYYRTSKAANRFSNLCSEDFLAFARQDFNICQAQHQKELQQLQRWYADCKLDTLKYGRDVVRVANFLTSAIIGDPELSDVRIVFAQHIVLVTRIDDFFDHRGSREESYKILELIKEWKEKPAAEYGSEEVEILFTAVYNTVNELAERAHVEQGRSVKDFLIKLWVQILSIFKRELDTWSDDTALTLDDYLSASWVSIGCRICILMSMQFIGIKLSDEMLLSEECIDLCRHVSMVDRLLNDVQTFEKERKENTGNSVTLLLAANKDDSSFTEEEAIRIAKEMAECNRRQLMQIVYKTGTIFPRQCKDMFLKVCRIGCYLYASGDEFTSPQQMMEDMKSLVYEPLTIHPLVANNVRGK

SEQ ID NO: 73 - protein, 300 aa, Synechococcus sp
MVVADAHTQGFSLAQYLQEQKTIVETALDQSLVITEPVTIYEAMRYSLLAGGKRLRPILCLAACEMLGGTAAMAMNTACALEMIHTMSLIHDDLPAMDNDDLRRGKPTNHKVYGEDIAILAGDALLSYAFEYVARTPDVPAERLLQVIVRLGQAVGAEGLVGGQVVDLESEGKTDVAVETLNFIHTHKTGALLEVCVTAGAILAGAKPEEVQLLSRYAQNIGLAFQIVDDILDITATAEELGKTAGKDLEAQKVTYPSLWGIEKSQAEAQKLVAEAIASLEPYGEKANPLKALAEYIVNR

SEQ ID NO: 74 - protein, 533 aa, Tripterygium wilfordii
MEFLLSLPTNTIAPKIFAVLLLFICLRILTNVLKPKKSKTSPPQASGAWPLIGHLLHLRGPQAPHITLGKMADKYGPIFKIKLGVHPTLVISDSEVAKECLTTHDIALAGRPATVAMEIMGYNHAMFAFSPYGPYWRHMRKLATVELLSAQRLETFKHIRESELKRSMKEMYQSWVHNKSGSGDSNHVTVDMTRILGDIIANVIYRMVVGKVYASKGEEDARWKQVVWEYIKLLSHFGVGDALPFLRWLDLGGVEKSMKKAAKELDIYVEEWLEEHKKKRSERKSDNGIVEEDFMDVMLSVFDDDDQLENFAHHSAHTINKAMCLAIILAASDTTKTTLTWALSLLLNHPDVMKKVQQELAAHIGPDKPVKESDVKSLVYLEAVVKETLRLYPPGPLGLPHESMEDCTVAGYHVPSGTRILYNLWKIQQDPQVWENPSEFKPDRFLTTHKDVDVRGRNFEYLPFGSGRRMCPGMSFALQVMEVSLANMLHGFDFATPNGKPVDMTEVNGLVTDRATPLEALITPRLPAHLYMG

SEQ ID NO: 75 - protein, 533 aa, Tripterygium wilfordii
MEFLLSLPTNTIATTSFAVLLLYLCLRIFTNVLKPKKSKTSPPQAGGAWPLIGHLHLLIGPQASYITLSKMADKYGPIFKIKLGVHPTLVISNSEVAKECLTTHDKVLANRPATVAMEIMGYNHAMFGWSPYGPYWRHMRKLATVELLSAQRLETFKHIRESELKRSMKEMYQSWVHNKSGSGDSNHVTVDMTRILGDIIANVIYRMVVGKVYASKGEEDARWKQVVWEYIKLLSHFGVGDALPFLRWLDLGGVEKSMKKAAKELDIYVEEWLEEHKKKRSERKSDNGIVEEDFMDVMLSVFDDDDQLENFAHHSAHTINKAMCLAIILAASDTTKTTLTWALSLLLNHPDVMKKVQQELAAHIGPDKPVKESDVKSLVYLEAVVKETLRLYPPGPLGLPHESMEDCTVAGYHVPSGTRILYNLWKIQQDPQVWENPSEFKPDRFLTTHKDVDVRGRNFEYLPFGSGRRMCPGMSFALQVMEVSLANMLHGFDFATPNGKPVDMTEVNGLVTDRATPLEALITPRLPAHLYMG

SEQ ID NO: 76 - protein, 530 aa, Tripterygium wilfordii
MEFLLSLPTNTIATKIFAVLLLYLFLRIFTNVLKPKKSKTSPPQAGGAWPLIGHLHLLIGPQASYITLSKMADKYGPIFKIKLGVHPTLVISNSEVAKECLTTHDKVLANRPATVAMEIMGYNHAMFGWSPYGPYWRQLRKLVTVELLSNQRLKTFKHIRESEVKNSLKEMYQSWVHNKSGDSNHVSVDMTRIFGDITGNLIYRIVVGKVYARKGEGVVRWKQVVGDYMKLLTHFNVGDAMPFMRWFDLGGLEKAMKITFKELDGYVEEWLEEHKKKRSNSGGHGIVEEDFMDVMLSIFDDGGQQEYCTDNSTHTTNKAMCMALILGASETTKTTLTWSLSLLLNNLDVLKKVKQELAAHIGPETLVTESDVNSLVYLDAVITETLRLYPLGPLGLPHESIEDCTIAGYHVPARTRILFNLWKIHQDPRVWENPLEFKPERFLKEHNNIDVRGGHFELLPFGSGRRMCPGVSFALQVLKLILANMLHGFDFATPNDEPVDMTEVNHMATTRATPLETLISPRLPSHLYMG

SEQ ID NO: 77 - protein, 749 aa, Plectranthus barbatus
MAWMNNGKNLNCQLTHKKISKVAEIRVATVNAPPVHDQDDSTENQCHDAVNNIEDPIEYIRTLLRTTGDGRISVSPYDTAWVALIKDLQGRDAPEFPSSLEWIIQNQLADGSWGDAKFFCVYDRLVNTIACVVALRSWDVHAEKVERGVRYINENVEKLRDGNEEHMTCGFEVVFPALLQRAKSLGIQDLPYDAPVIQEIYHSREQKSKRIPLEMMHKVPTSLLFSLEGLENLEWDKLLKLQSADGSFLTSPSSTAFAFMQTRDPKCYQFIKNTIQTFNGGAPHTYPVDVFGRLWAIDRLQRLGISRFFESEIADCIAHIHRFWTEKGVFSGRESEFCDIDDTSMGVRLMRMHGYDVDPNVLKNFKKDDKFSCYGGQMIESPSPIYNLYRASQLRFPGEQILEDANKFAYDFLQEKLAHNQILDKWVISKHLPDEIKLGLEMPWYATLPRVEARYYIQYYAGSGDVWIGKTLYRMPEISNDTYHELAKTDFKRCQAQHQFEWIYMQEWYESCNMEEFGISRKELLVAYFLATASIFELERANERIAWAKSQIISTIIASFFNNQNTSPEDKLAFLTDFKNGNSTNMALVTLTQFLEGFDRYTSHQLKNAWSVWLRKLQQGEGNGGADAELLVNTLNICAGHIAFREEILAHNDYKTLSNLTSKICRQLSQIQNEKELETEGQKTSIKNKELEEDMQRLVKLVLEKSRVGINRDMKKTFLAVVKTYYYKAYHSAQAIDNHMFKVLFEPVA

SEQ ID NO: 78 - protein, 567 aa, Plectranthus barbatus
MITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 79 - protein, 722 aa, Plectranthus barbatus
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 80 - protein, 525 aa, Saccharomyces cerevisiae
MDQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 81 - protein, 297 aa, Synechococcus sp
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

(19) United States
(12) Patent Application Publication
(10) Pub. No.: US 2025/0263763 A1
(43) Pub. Date: Aug. 21, 2025

(54) PRODUCTION OF OXYGENATED DITERPENOID COMPOUNDS

(71) Applicant: Københavns Universitet, Copenhagen K (DK)

(72) Inventors: Johan Andersen-Ranberg, København K (DK); Nikolaj Hansen, Farum (DK); Victor Forman, København N (DK)

(21) Appl. No.: 18/022,026

(22) PCT Filed: Aug. 26, 2021
(86) PCT No.: PCT/EP2021/073656

§ 371 Date: Feb. 17, 2023

(30) Foreign Application Priority Data
Aug. 27, 2020 (DK) PA 2020 00864

(51) Int. Cl. C12P 17/18; C07C 35/17; C07C 35/42; C07C 39/17; C07C 39/23; C07C 47/46; C07C 50/34; C07C 69/08; C07D 209/62; C07D 303/14; C07D 307/92; C07D 493/04; C12N 9/02

(52) U.S. Cl. CPC C12P 17/181; C07C 35/17; C07C 35/42; C07C 39/17; C07C 39/23; C07C 47/46; C07C 50/34; C07C 69/08; C07D 209/62; C07D 303/14; C07D 307/92; C07D 493/04; C12N 9/0071; C07C 2601/14; C07C 2602/28; C07C 2603/26

(57) ABSTRACT

Disclosed is a method for production of oxygenated diterpenoid compounds, such as triptophenolide, triptonide and triptolide, by inserting genes encoding particular cytochrome P450 enzymes and expressing the genes in selected host cells for synthesis of the compounds. Further disclosed are particular cytochrome P450 enzymes suitable for this synthesis.

### REFERENCE TO SEQUENCE LISTING

The present application contains a sequence listing in computer readable form, which is incorporated by reference.

### FIELD OF INVENTION

The invention relates to the production of oxygenated diterpenoid compounds in recombinant cells, such as yeast cells. The oxygenated diterpenoid compounds are useful as intermediate or final compounds in the synthesis of useful bioactive compounds for use in e.g. pharmaceutical treatment of diseases such as cancer. The invention further relates to genes, enzymes and cells, such as yeast cells, particularly suited for production of such compounds.

### BACKGROUND FOR THE INVENTION

Terpenes is a diverse group of compounds generated from a basic 5-carbon structure, isoprene (2-methyl-1,3-butadiene). Diterpenes are compounds having a 20-carbon structure, generated by the action of an enzyme, diterpene synthase, that converts the compound geranyl-geranyl-diphosphate (GGPP) into a diterpene structure, which can by further modified forming a vast range of diterpene or diterpenoid compounds. Diterpenes, diterpenoids, derivatives thereof are widely used, e.g. as pharmaceuticals, cosmetics, nutraceuticals, flavors, fragrances and pesticides. Methods for increasing the production of these compounds in natural or engineered cells are abundant in the art. The Chinese medicinal plant, Tripterygium wilfordii, is known to produce several sesquiterpenoids, diterpenoids and triterpenoids with potential pharmacological properties, including the diterpenoid compounds triptonide and triptolide. Triptolide, an oxygenated diterpenoid compound, and derivatives thereof, has been identified as potential valuable pharmacological compounds and is under investigation as immunosuppressant and for treatment of cancer. Triptolide may further be used in treatment against COVID-19. Triptonide may be useful as male contraceptive agent.

Using engineered microorganisms for producing valuable molecules from renewable feedstock is a desirable alternative from conventional means of production. However, achieving economically viable yield, titers and productivity is a major roadblock towards industrialisation.

N L Hansen et al (2017) in The Plant Journal 2017, 89, 429-441, described a diterpene synthase capable of converting GGPP into the dipterpene, miltiradiene, which is a precurser for triptolide. The findings were confirmed by P Su et al (2018) in The plant Journal 2018, 50-65; and by J Guo et al. PNAS 2013, 110, 12108-12113.

The complete pathway for converting miltiradiene into other diterpenoid compounds, such as triptolide, has not yet been elucidated.

Cytochrome P450 enzymes (CYPs) are involved in the biosynthesis of terpenoids, and for many cytochrome P450 enzymes nothing is known with respect to the substrate they are acting on, which compounds they are generating, or their role in the biosynthesis of specific compounds.

US20190270971A1 discloses methods for increasing productivity of microbial host cells that functionally express p450 enzymes. The document describes how P450 genes can be modified in order to improve performance in microorganisms, such as yeast, and it mentions that co-expression with cytochrome P450 reductase can be beneficial to improve the yield. It is mentioned that triptolide may be the subject of P450 chemistry, but the document does not provide any link between triptolide and any specific P450 enzymes or cytochromes.

CN 108395997A describes yeast with increased GGPP production. The yeast is transformed with different diterpene synthases and P450 enzymes to synthesize diterpenoid compounds. The scientist team behind this patent is also behind more patents and patent applications disclosing the synthesis of different di- and triterpenoid compounds using suitable terpene synthases and P450 enzymes e.g. CN 108866029 (friedelin), CN107058419 (Kauren-type) and WO 2020029564 (Fridelin and amyrins). CN 110747178A describes the P450 gene TwCYP728B70 as encoding a Cytochrome P450 enzyme having a role in triptolide synthesis.

### SHORT DESCRIPTION OF THE INVENTION

The inventors have solved the problem of providing an improved method of producing oxygenated diterpenoid compounds, such as triptophenolide, triptonide and triptolide. In a first aspect the invention relates to a method for producing an oxygenated diterpenoid compound is disclosed, the method comprising the steps of:
a. providing a host cell capable of producing miltiradiene and/or dehydroabietadiene;
b. transforming the host cell with a first gene encoding an enzyme having cytochrome P450 activity;
c. growing the transformed cell under conditions leading to expression of the transformed gene; whereby the oxygenated diterpenoid compound is formed;
   wherein:
the first gene encoding an enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 1 (TwCYP82D274v1), SEQ ID NO: 2 (TwCYP82D274v2), SEQ ID NO: 74 (TwCYP82D274v3) or SEQ ID NO: 75 (TwCYP82D274v4), or the mature polypeptide thereof.

In a second aspect, the invention relates to methods for producing oxygenated diterpenoid compound, and comprises transforming the host cell with the first gene encoding an enzyme having cytochrome P450 activity and further with a second gene encoding a second enzyme having cytochrome P450 activity and with a third gene encoding a third enzyme having cytochrome P450 activity wherein:
the second gene encoding an enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 3 (TwCYP71BE85) or the mature polypeptide thereof; and
the third gene encoding an enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 4 (TwCYP71BE85) or the mature polypeptide thereof.

In a third aspect, the invention relates to methods for producing oxygenated diterpenoid compound, wherein said method comprises that the host cell is transformed with the first, second and third genes encoding enzymes having cytochrome P450 activities, and further with a fourth gene encoding a fourth enzyme having cytochrome P450 activity;
wherein:
   the fourth gene encoding a fourth enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 5 (TwCYP82D213v1) or to SEQ ID NO: 76 (TwCYP82D213v2), or the mature polypeptide thereof.

According to the invention the useful oxygenated diterpenoid compounds, triptophenolide, triptonide or triptolide, may be provided.

The invention further relates to polypeptides, polynucleotides, plasmids and expression constructs as well as recombinant host cells useful in the methods of the invention.

### SHORT DESCRIPTION OF THE DRAWINGS

FIG. 1 shows LCMS profiles of extracts from N. benthamiana leafs expressing miltirdiene biosynthesis genes and selected T. wilfordii CYPs.

TwCYPs was co-expressed with CfDXS, CfGGPPS, CfTPS1 and CfTPS3. “3×STD” represents a LCMS run of a sample of three mixed authentic standards: Triptolide, triptonide, and triptophenolide. Solid lines represent ion chromatograms at range m/z 280-380. Dashed lines (- ---- -) represent extracted ion chromatograms at m/z 313.1800±0.015 which corresponds to the parental ion of triptophenolide [M+H]. Dashed lines (- - -) represents extracted ion chromatograms at m/z 359.1490±0.0 15 which corresponds to the parental ion of triptonide [M+H]. LC protocol 1 used. For more details see example 1.

FIG. 2 shows LCMS profiles of extracts of genetically engineered Saccharomyces cerevisiae (S. cerevisiae) strain.

In the background strain (-), genes encoding the diterpene biosynthetic enzymes SPGGPPS7, CftTPS1, CftTPS3 and TwCPR1 were integrated into genome of wild type S. cerevisiae. In the TwCYP82D274v1 strain, the diterpene biosynthetic enzymes were expressed with TwCYP82D274v1, resulting in the formation of compound (3) identified as 14-OH-dehydroabietadiene marked in grey. LC method 1 was used for the analysis. For more details see example 3.

FIG. 3 shows the ¹H NMR spectrum of 14-OH-dehydroabietadiene in CDCl₃ at 599.85 MHz. For more details see example 4.

FIG. 4 shows the ¹³C NMR spectrum of 14-OH-dehydroabietadiene in CDCl₃ at 150.83 MHz. For more details see example 4.

FIG. 5 shows LCMS profiles of extracts of yeast having denoted gene combinations genome integrated.

All yeast strains have genome integrated spGGPPs7, CftTPS1, CftTPS3 and TwCPR1. “0.5 ppm 3×STD” represents a LCMS run of a sample of three mixed authentic standards: Triptolide, triptonide and triptophenolide. Non-dashed lines represent ion chromatograms at range m/z 280-380. Dashed lines represents extracted ion chromatograms at m/z 359.1490±0.015 which corresponds to the parental ion of triptonide ([M+H]⁺). LC protocol 2 used. For more details see example 5.

FIG. 6 shows co-expression of TwCYPs and different variants of B5 proteins isolated from Tripterygium wilfordii

Levels of triptophenolide, triptonide and 14-OH-dehydroabietadiene quantified from cultures of engineered S. cerevisiae strains. Each column represents an engineered yeast strain and their output of selected compounds. The genes integrated into each of the individual strains are denoted in the lower panel. Quantification was based on the areas of the peaks representing each of the compounds of interest. Individual scales applies for each of the compounds. For more details see example 5.

FIG. 7 shows relative quantity (bars) of key intermediates in the proposed biosynthetic pathway of triptonide, when established in vivo, via heterologous gene expression in N. benthamiana (panel A and D) and S. cerevisiae (panel B and E, strains listed in table 3). Gene expression is indicated by black squares to the left, while relative quantity is indicated by bars (average of 3-4 biological replicates; black diamond squares) with white- and grey fill color distinguishing expression and no expression of Twb5 #1, respectively. Error bars represent standard deviation. “DiTPSs” reflects CftTPS1 and CftTPS3. In quantification of peak areas, the signature mass tolerance was ±0.1m/z for GCMS (miltiradiene and 14-OH-dehydroabietadiene) and ±0.005m/z for LCMS (all other compounds). Panel C: A hypothesized biosynthetic pathway from miltiradiene to triptonide in vivo in N. benthamiana and S. cerevisiae that include a Wagner-Meerwein rearrangement reaction to account for a methyl shift of C-19 or C-18 to C-3 in the abietane carbon backbone.

FIG. 8 shows accumulation over 7 days of triptophenolide and triptonide produced with yeast strain NVJ8.15 when grown in bioreactor. Level of triptonide (solid black line) and triptophenolide (dotted black line) shows absolute quantity (ppm, w/v) in samples of the culture taken each day. Biomass was quantified by absorbance at 600 nm (grey dotted line).

FIG. 9 shows yeast strains expressing the genes needed for triptonide biosynthesis, but with genes variants substituting TwCYP82D274v1 or TwCYP82D213v1, pertain the ability to produce triptophenolide (panel A) and triptonide (panel B) and results in similar LCMS profiles (panel C). Genes present in the engineered strains are represented by the black squares. Panel A and B: Bars represent the average relative quantity (2-3 biological replicates, crosses) with error bars showing std. error. From left to right, bars represent yeast strains: NVJ10-1, NVJ10-3, NVJ10-6, NVJ10-8 (see table 3). Panel C: EICs (m/z 280-360) of LCMS analyzed yeast cultures. From top and down pairs of chromatograms represent yeast strains NVJ10-1, NVJ10-3, NVJ10-6 and NVJ10-8.

### SHORT DESCRIPTION OF THE SEQUENCES

SEQ ID NO: 1 shows the amino acid sequence of a cytochrome P450 enzyme derived from T. wilfordii. The enzyme is also known as TwCYP82D274v1.

SEQ ID NO: 2 shows the amino acid sequence of a cytochrome P450 enzyme derived from T. wilfordii. The enzyme is also known as TwCYP82D274v2. SEQ ID NO: 2 differs from SEQ ID NO: 1 in only three positions, and it is therefore assumed that SEQ ID NO: 1 and SEQ ID NO: 2 represent different alleles of the same gene.

SEQ ID NO: 3 shows the amino acid sequence of a cytochrome P450 enzyme derived from T. wilfordii. The enzyme is also known as TwCYP71BE85.

SEQ ID NO: 4 shows the amino acid sequence of a cytochrome P450 enzyme derived from T. wilfordii. The enzyme is also known as TwCYP71BE86.

SEQ ID NO: 5 shows the amino acid sequence of a cytochrome P450 enzyme derived from T. wilfordii. The enzyme is also known as TwCYP82D213v1.

SEQ ID NO: 6 shows the amino acid sequence of a cytochrome P450 enzyme derived from T. wilfordii. The enzyme is also known as TwCYP82D217.

SEQ ID NO: 7 shows the amino acid sequence of a cytochrome P450 enzyme derived from T. wilfordii. The enzyme is also known as TwCYP82D275.

SEQ ID NO: 8 shows the amino acid sequence of a cytochrome B5 enzyme derived from T. wilfordii. This enzyme is also known as TwB5 #1.

SEQ ID NO: 9 shows the amino acid sequence of a cytochrome P450 reductase enzyme derived from T. wilfordii. This enzyme is also known as TwCPR1.

SEQ ID NO: 10-66 show PCR primers as further described in Example 2.

SEQ ID NO: 67 shows the amino acid sequence of a diterpene synthase TPS1 derived from Plectranthus barbatus. The enzyme is also known as CfTPS1.

SEQ ID NO: 68 shows the amino acid sequence of a diterpene synthase TPS3 derived from Plectranthus barbatus. The enzyme is also known as CfTPS3.

SEQ ID NO: 69 shows the amino acid sequence of a terpene synthase TPS9, derived from T. wilfordii. The enzyme is also known as TwTPS9.

SEQ ID NO: 70 shows the amino acid sequence of a terpene synthase derived from T. wilfordii. The enzyme is also known as TwTPS27.

SEQ ID NO: 71 shows the amino acid sequence of a copalyl diphosphate synthase CPS1 derived from Salvia miltiorrhiza. The enzyme is also known as SmCPS.

SEQ ID NO: 72 shows the amino acid sequence of a miltiradiene synthase KSL1 derived from Salvia miltiorrhiza. The enzyme is also known as SmKSL.

SEQ ID NO: 73 shows the amino acid sequence of a geranyl geranyl diphosphate synthase derived from Synechococcus sp. The enzyme is also known as SpGGPPs7v1.

SEQ ID NO: 74 shows the amino acid sequence of a cytochrome P450 enzyme derived from T. wilfordii. The enzyme is also known as TwCYP82D274v3.

SEQ ID NO: 75 shows the amino acid sequence of a cytochrome P450 enzyme derived from T. wilfordii. The enzyme is also known as TwCYP82D274v4.

SEQ ID NO: 76 shows the amino acid sequence of a cytochrome P450 enzyme derived from T. wilfordii. The enzyme is also known as TwCYP82D213v2.

SEQ ID NO: 77 shows the truncated amino acid sequence of a diterpene synthase TPS1 derived from Plectranthus barbatus. The amino acid sequence was truncated to remove a transit peptide. The enzyme is also known as CftTPS1.

SEQ ID NO: 78 shows the truncated amino acid sequence of a diterpene synthase TPS3 derived from Plectranthus barbatus. The amino acid sequence was truncated to remove a transit peptide. The enzyme is also known as CftTPS3.

SEQ ID NO: 79 shows the amino acid sequence of a DXS enzyme derived from Plectranthus barbatus. The enzyme is also known as CfDXS.

SEQ ID NO: 80 shows the amino acid sequence of a truncated HMGR enzyme derived from S. cerevisiae. The enzyme is also known as SctHMGR.

SEQ ID NO: 81 shows the amino acid sequence of a geranyl geranyl diphosphate synthase derived from Synechococcus sp. The enzyme is also known as SpGGPPs7v2.

### DETAILED DESCRIPTION OF THE INVENTION

According to a first aspect of the invention, a method for producing an oxygenated diterpenoid compound is disclosed, the method comprising the steps of:
a. providing a host cell capable of producing miltiradiene and/or dehydroabietadiene;
b. transforming the host cell with a first gene encoding an enzyme having cytochrome P450 activity;
c. growing the transformed cell under conditions leading to expression of the transformed gene; whereby the oxygenated diterpenoid compound is formed;
   wherein:
the first gene encoding an enzyme having cytochrome P450 activity encodes a polypeptide comprising or consisting of SEQ ID NO:1 or an amino acid sequence having at least 80% sequence identity, preferably at least 85% sequence identity, preferably at least 90% sequence identity, preferably at least 95% sequence identity more preferred at least 98% sequence identity to SEQ ID NO: 1 (TwCYP82D274V1) or the mature polypeptide thereof.

The polypeptide having SEQ ID NO: 1 is a preferred example of a first gene having cytochrome P450 activity, the polypeptides having SEQ ID NO: 2, SEQ ID NO: 74 and SEQ ID NO: 75 are other examples of such a polypeptide.

Thus, the enzyme encoded by the first gene has the ability to convert miltiradiene and/or dehydroabietadiene into 14-OH-dehydroabietadiene by inserting an OH group in position 14 of the diterpene skeleton of miltiradiene.

In some embodiments, the synthesis of 14-OH-dehydroabietadiene takes place via the compound 14-OH-miltiradiene that subsequently is converted into 14-OH-dehydroabietadiene. However, the invention is not limited to any particular mechanism for converting miltiradiene into 14-OH-dehydroabietadiene.

Using the method according to the first aspect of the invention leads to the formation of the oxygenated diterpenoid compound, 14-OH-dehydroabietadiene,

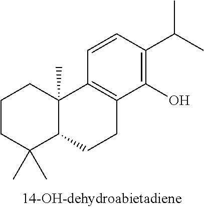 [SMILES: CC(C)c1ccc2c(c1O)CC[C@@H]1C(C)(C)CCC[C@@]21C]

that is a useful intermediate in the synthesis of oxygenated diterpenoid compounds of pharmaceutical use, including well known compounds such as triptophenolide, triptonide and triptolide.

In a second aspect of the invention, the method step b. comprises transforming the host cell with the first gene encoding an enzyme having cytochrome P450 activity and further with a second gene encoding a second enzyme having cytochrome P450 activity and a third gene encoding a third enzyme having cytochrome P450 activity
wherein:
   the second gene encoding an enzyme having cytochrome P450 activity encodes a polypeptide comprising or consisting of SEQ ID NO:4 or an amino acid sequence having at least 80% sequence identity, preferably at least 85% sequence identity, preferably at least 90% sequence identity, preferably at least 95% sequence identity, more preferred at least 98% sequence identity to SEQ ID NO: 4 (TwCYP71BE86) or the mature polypeptide thereof; and
   the third gene encoding an enzyme having cytochrome P450 activity encodes a polypeptide comprising or consisting of SEQ ID NO: 3 or an amino acid sequence having at least 80% sequence identity, preferably at least 85% sequence identity, preferably at least 90% sequence identity, preferably at least 95% sequence identity, more preferred at least 98% sequence identity to SEQ ID NO: 3 (TwCYP71BE85) or the mature polypeptide thereof.

In this second aspect, the host cell preferably further produces the oxygenated diterpenoid compound, triptophenolide, (3bR,9bS)-6-hydroxy-9b-methyl-7-propan-2-yl-3,3b,4,5,10,11-hexahydronaphtho[2,1-e]isobenzofuran-1-one.

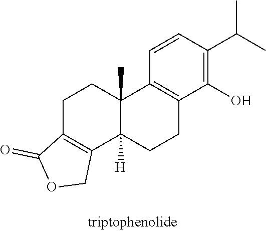 [SMILES: CC(C)c1ccc2c(c1O)CC[C@H]1C3=C(CC[C@]21C)C(=O)OC3]

Triptophenolide is a valuable compound that has been identified as an antiandrogen. In addition, it may be useful as a starting point for further modifications leading to further bioactive compounds.

In a preferred embodiment of the second aspect of the invention, the host cell is further transformed with a fifth gene encoding a polypeptide having cytochrome B5 activity and comprising or consisting of SEQ ID NO:8 or an amino acid sequence having at least 80% sequence identity, preferably at least 85% sequence identity, preferably at least 90% sequence identity, preferably at least 95% sequence identity, more preferred at least 98% sequence identity to SEQ ID NO: 8 (TwB5 #1) or the mature polypeptide thereof. It has surprisingly been found that expressing the polypeptide having cytochrome B5 activity in the same cell that expresses the first, second and third genes encoding enzymes having cytochrome P450 activity, leads to a significantly higher production of the oxygenated diterpenoid compound. The production is increased at least 50% compared with the production of a similar cell without the polypeptide having cytochrome B5 activity, preferably increased at least 100%, preferably at least 200% or even more.

In a third aspect of the invention, the host cell is transformed with the first, second and third genes encoding enzymes having cytochrome P450 activities, and further with a fourth gene encoding a fourth enzyme having cytochrome P450 activity;
wherein:
   the fourth gene encoding a fourth enzyme having cytochrome P450 activity encodes a polypeptide comprising or consisting of SEQ ID NO:5 or an amino acid sequence having at least 80% sequence identity, preferably at least 85% sequence identity, preferably at least 90% sequence identity, preferably at least 95% sequence identity, more preferred at least 98% sequence identity to SEQ ID NO: 5 (TwCYP82D213v1) or the mature polypeptide thereof. The polypeptide having SEQ ID NO: 5 is a preferred example of a fourth gene having cytochrome P450 activity; the polypeptide having SEQ ID NO: 76 is another example of such a polypeptide.

In the third aspect of the invention, the transformed eukaryotic cell preferably produces the oxygenated diterpenoid compound, triptonide.

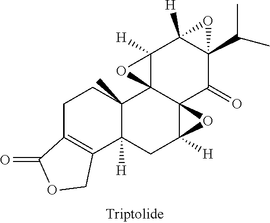 [SMILES: CC(C)[C@]12O[C@H]1[C@@H]1O[C@@]13[C@@]1(C)CCC4=C(COC4=O)[C@@H]1C[C@@H]1O[C@@]13C2=O]

The compound triptonide has been reported to have a strong inhibition activity in cancers (Fulu Dong et al 2019, The Prostate, Volume 19, issue 11, pages 1284-1293). The compound is also useful as male contraceptive agent. Further, the compound is useful as a starting point for further modifications leading to further bioactive compounds.

In a preferred embodiment of the third aspect of the invention, the host cell is further transformed with a fifth gene encoding a polypeptide having cytochrome B5 activity and comprising or consisting of SEQ ID NO:8 or an amino acid sequence having at least 80% sequence identity, preferably at least 85% sequence identity, preferably at least 90% sequence identity, preferably at least 95% sequence identity, more preferred at least 98% sequence identity to SEQ ID NO: 8 (TwB5 #1) or the mature polypeptide thereof. It has surprisingly been found that expressing the polypeptide having cytochrome B5 activity in the same cell that expresses the first, second, third and fourth genes encoding enzymes having cytochrome P450 activity, leads to a significantly higher production of the oxygenated diterpenoid compound. The production is increased at least 50% compared with the production of a similar cell without the polypeptide having cytochrome B5 activity, preferably increased at least 100%, preferably at least 200% or even more.

In a further preferred embodiment of the third aspect of the invention, the host cell is further transformed with a sixth gene encoding a fifth enzyme having cytochrome P450 activity and/or a seventh gene encoding a sixth enzyme having cytochrome P450 activity wherein:
the sixth gene encoding a fifth enzyme having cytochrome P450 activity encodes a polypeptide comprising or consisting of SEQ ID NO:6 or an amino acid sequence having at least 80% sequence identity, preferably at least 85% sequence identity, preferably at least 90% sequence identity, preferably at least 95% sequence identity, more preferred at least 98% sequence identity to SEQ ID NO: 6 (TwCYP82D217) or the mature polypeptide thereof; and
the seventh gene encoding a sixth enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 7 (TwCYP82D275) or the mature polypeptide thereof. It has surprisingly been found that expressing the sixth and/or seventh genes encoding enzymes having cytochrome P450 activity, leads to a higher production of the oxygenated diterpenoid compound. Preferably, the production is increased at least 10% compared with the production of a similar cell without sixth and/or seventh genes encoding enzymes having cytochrome P450 activity, preferably increased at least 20%, even more preferred at least 50% or even more.

The first, second, third, fourth, fifth, sixth and seventh gene may be comprised in one or more nucleic acid molecules, such as one or more heterologous nucleic acids. The heterologous nucleic acid encoding the first enzyme having cytochrome P450 activity may herein be referred to as the “first heterologous nucleic acid”. The heterologous nucleic acid encoding the second enzyme having cytochrome P450 activity may herein be referred to as the “second heterologous nucleic acid”. The heterologous nucleic acid encoding the third enzyme having cytochrome P450 activity may herein be referred to as the “third heterologous nucleic acid”. The heterologous nucleic acid encoding the fourth enzyme having cytochrome P450 activity may herein be referred to as the “fourth heterologous nucleic acid”. The heterologous nucleic acid encoding the fifth enzyme having cytochrome P450 activity may herein be referred to as the “fifth heterologous nucleic acid”. The heterologous nucleic acid encoding the sixth enzyme having cytochrome P450 activity may herein be referred to as the “sixth heterologous nucleic acid”. The heterologous nucleic acid encoding the enzyme having cytochrome B5 activity may herein be referred to as the “seventh heterologous nucleic acid”. This does not imply that the recombinant host cell must comprises seven heterologous nucleic acids in total; in some embodiments, the cell comprises only one or more of the first, second, third, fourth, fifth, sixth and seventh heterologous nucleic acids.

The oxygenated diterpenoid compounds produced according to the methods of the invention may be further modified by biological or chemical synthesis. In connection with this, biological synthesis is understood as a method where the host cell comprising the genes of the invention is further provided with one of more additional genes encoding further enzymes having the capability of modifying the oxygenated diterpenoid compounds produced according to the methods of the invention.

Chemical modification of the oxygenated diterpenoid compounds produced according to the methods of the invention may be performed directly on the culture broth before recovery of the oxygenated diterpenoid compounds or it may be performed on the recovered oxygenated diterpenoid compounds.

Reduction of triptonide to triptolide can be achieved by organic synthesis. An example of such synthesis is the reduction by a nucleophilic attack by a hydride on C-14 ketone. For this reaction Sodium borohydride is a suitable agent for catalyzing this reaction at neutral pH in the appropriate solvent e.g. water or MetOH.

In one preferred embodiment, triptonide produced according to the methods of the invention is converted into the compound triptolide, that is reported to be an immunosuppressant and is under investigation for use in cancer therapy.

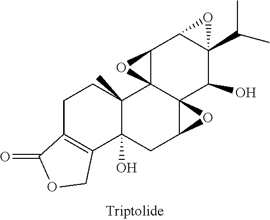 [SMILES: CC(C)[C@]12O[C@H]1[C@@H]1O[C@]13[C@]1(O[C@H]1C[C@@]1(O)C4=C(CC[C@]31C)C(=O)OC4)[C@@H]2O]

### The Host Cell

The host cell capable of producing miltiradiene and/or dehydroabietadiene may in principle be any such cell. The cell may be a cell that naturally produces miltiradiene and/or dehydroabietadiene or it may be a cell that has been engineered to produce one or both of these compounds.

It is believed that miltiradiene, at least under some circumstances, may be spontaneously converted into dehydroabietadiene, and the invention may therefore be performed using a cell producing miltiradiene that is spontaneously converted into dehydroabietadiene or it may be performed in a cell comprising an enzyme that facilitates the conversion of miltiradiene to dehydroabietadiene (see a J. Zi, et al., Organic & Biomolecular Chemistry 2013, 11, 7650-7652).

The synthesis of miltiradiene in general begins with the formation of GGPP. GGPP may be synthesized by condensation of one dimethylallyl pyroophosphate (DMAP) molecule and three isopentenyl pyrophosphate (IPP) molecules and is typically catalyzed by a geranylgeranyl diphosphate synthase e.g. the SpGGPPs7 enzyme derived from Synechococcus sp.; and having the amino acid sequence shown in SEQ ID NO: 73 or SEQ ID NO: 81.

GGPP is converted into miltiradiene by action of a diterpene synthase or by the combined action of two or more diterpene synthases, e.g. a combination of two diterpene synthases, CfTPS1 and CfTPS3, or CftTPS1 as set forth in SEQ ID NO: 77 and CftTPS3 as set forth in SEQ ID NO: 78, derived from Plectranthus barbatus and having the amino acid sequences of SEQ ID NO: 67 and 68; a combination of two diterpene synthases, TwTPS9 and TwTPS27, derived from T. wilfordii and having the amino acid sequences of SEQ ID NO: 69 and 70; or a combination of a copalyl diphosphate synthase, SmCPS derived from Salvia miltiorrhiza and having the amino acid sequence of SEQ ID NO: 71, and a miltiradiene synthase, SmKSL derived from Salvia miltiorrhiza and having the amino acid sequence of SEQ ID NO: 72.

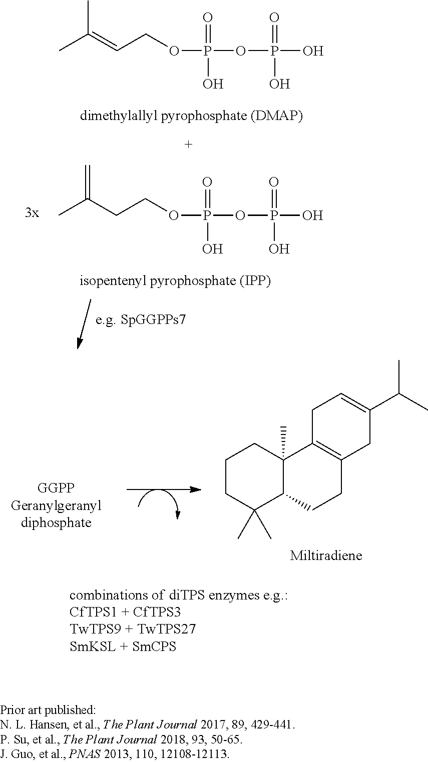

One preferred way to provide a host cell producing miltiradiene and/or dehydroabietadiene is selecting a host cell producing GGPP and transforming it with a diterpene synthase catalyzing the transformation of GGPP into miltiradiene. Alternatively, a host cell that have been genetically engineered to produce GGPP may be used as a starting point. Techniques for transforming a host cell with a diterpene synthase catalyzing the transformation of GGPP into miltiradiene is known in the prior art, e.g. in NL Hansen et al (2017) in The Plant Journal 2017, 89, 429-441 (Incorporated herein by reference), P Su et al (2018) in The plant Journal 2018, 50-65 and in: J. Guo, et al., Proceedings of the National Academy of Sciences 2013, 110, 12108-12113, and the procedures and methods disclosed in these publications are also useful for providing a host cell for use in the present invention.

The host cell may be a prokaryotic cell, such as a eubacterial or archaebacterial cell; or a eukaryotic cell, such as a plant cell, an animal cell, an insect cell, a fungal cell or a yeast cell.

Practically all eukaryotic cells produce GGPP for their biosynthesis, but in some embodiments a eukaryotic cell produces an increased amount of GGPP, which may increase the production of miltiradiene, compared with a similar eukaryotic that does not produce increased amounts of GGPP. Methods for increasing the GGPP production in a eukaryotic cell has also been described in the prior art.

The host cell may be a unicellular organism, or it may be comprised within a multicellular organism, e.g. a plant. Examples of suitable plants or plant cells for use as host cells according to the invention includes corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annuas), wheat (Tritium aestivum and other species), triticale, rye (Secale) soybean (Glycine max), tobacco (Nicotiana tabacum), potato (Solanum tuberosum), peanuts (Arachis hypogaea), cotton (Gossypium hirsutum), sweet potato (Impomoea batatus), cassava (Manihot esculenta), coffee (Coffea spp.), coconut (Cocos nucifera), pineapple (Anana comosus), citrus (Citrus spp.) cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Primus amygdalus), apple (Malus spp.), pear (Pyrus spp.), plum and cherry tree (Prunus spp.), ribes (currant etc.), Vitis, Jerusalem artichoke (Helianthemum spp.), non-cereal grasses (Grass family), sugar and fodder beets (Beta vulgaris), chicory, oats, barley, vegetables, or ornamentals, crop plants (for example, cereals and pulses, maize, wheat, potatoes, tapioca, rice, sorghum, millet, cassava, barley, pea, sugar beets, sugar cane, soybean, oilseed rape, sunflower and other root, tuber or seed crops. Other important plants may be fruit trees, crop trees, forest trees or plants grown for their use as spices or pharmaceutical products (Mentha spp., clove, Artemesia spp., Thymus spp., Lavendula spp., Allium spp., Hypericum, Catharanthus spp., Vinca spp., Papaver spp., Digitalis spp., Rawolfia spp., Vanilla spp., Petrusilium spp., Eucalyptus, tea tree, Picea spp., Pinus spp., Abies spp., Juniperus spp. Horticultural plants which can be used with the present invention may include lettuce, endive, and vegetable brassicas including cabbage, broccoli, and cauliflower, carrots, and carnations and geraniums.

The plant can also be tobacco, cucurbits, carrot, strawberry, sunflower, tomato, pepper, or chrysanthemum.

Further examples of plants include grain plants for example oil-seed plants or leguminous plants. Seeds of interest include grain seeds, such as corn, wheat, barley, sorghum, rye, etc. Oil-seed plants include cotton soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea.

Particular preferred plant species include Physcomitrella sp., such as P. patens; Arabidopsis sp., such as A. thaliana; Nicotiana sp., such as N. benthamiana; Chlamydomonas sp., such as C. reinhardtii; and Nannochloropsis sp., such as N. oceanica.

Examples of suitable eukaryotic cell for use according to the invention include fungal cells such as Agaricus, Aspergillus, Candida, Eremothecium, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments, a host cell can be an Ascomycete such as Gibberella fujikuroi, Kluyveromyces lactis, Schizosaccharomyces pombe, Aspergillus niger, Yarrowia lipolytica, Ashbya gossypii, or S. cerevisiae.

In some embodiments, the host cell can be an algae cell such as Blakeslea trispora, Dunaliella salina, Haematococcus pluvialis, Chlorella sp., Undaria pinnatifida, Sargassum, Laminaria japonica, Scenedesmus almeriensis.

In some embodiments, a host cell can be a prokaryote such as Bacillus cells, for example Bacillus subtilis; Escherichia cells, for example, Escherichia coli cells; Lactobacillus cells; Lactococcus cells; Streptomyces cells, Streptococcus cells, Cornebacterium cells; Acetobacter cells; Acinetobacter cells; or Pseudomonas cells.

In some embodiments, the host cell can be a cyanobacterial cell such as Synechocystis sp. or Synechococcus sp.

In one embodiment, a host cell that is suitable for growth in a fermenter is selected. Growing the recombinant host cell according to the invention is a convenient way of growing the host cell for production of the oxygenated diterpenoid compounds of the invention.

In another embodiment, the host cell is a phototropic cell and the cell is cultivated in a green house or photobioreactor.

### The Genes and Enzymes

The recombinant host cell of the present invention is capable of producing miltiradiene and/or dehydroabietadiene. Miltiradiene may be spontaneously converted into dehydroabietadiene or it may be converted by an enzyme that facilitates the conversion of miltiradiene to dehydroabietadiene.

As described herein above, the synthesis of miltiradiene usually begins with the formation of GGPP by condensation of one dimethylallyl pyroophosphate (DMAP) molecule and three isopentenyl pyrophosphate (IPP) molecules by a geranylgeranyl diphosphate synthase.

Recombinant host cells and heterologous nucleic acids that encode enzymes that catalyze the synthesis of GGPP in recombinant host cells are generally known in the art, see e.g. WO 2015/113570. In addition, many host organisms are capable of producing GGPP intrinsically and heterologous nucleic acids may thus not always be necessary for production of GGPP.

In some embodiments, the recombinant host cell comprises a heterologous nucleic acid encoding a geranylgeranyl diphosphate synthase, such as the geranylgeranyl diphosphate synthase SpGGPPs7 as set forth in SEQ ID NO: 73 or SEQ ID NO: 81, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof.

Subsequently, GGPP may be converted into miltiradiene by the action of one or more diterpene synthases, copalyl diphosphate synthases and/or miltiradiene synthases.

In some embodiments, the recombinant host cell comprises one or more heterologous nucleic acids encoding one or more diterpene synthases, such as the diterpene synthases CfTPS1 (SEQ ID NO: 67) and CfTPS3 (SEQ ID NO: 68), or CftTPS1 (SEQ ID NO: 77) and CftTPS3 (SEQ ID NO: 78), or respective functional homologues thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptides thereof.

In some embodiments, the recombinant host cell comprises one or more heterologous nucleic acids encoding one or more diterpene synthases, such as the diterpene synthases TwTPS9 (SEQ ID NO: 69) and TwTPS27 (SEQ ID NO: 70), or respective functional homologues thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptides thereof.

In some embodiments, the recombinant host cell comprises a combination of one or more copalyl diphosphate synthases and one or more miltiradiene synthases, such as a combination of the copalyl diphosphate synthases SmCPS (SEQ ID NO: 71) and the miltiradiene synthase SmKSL (SEQ ID NO: 72), or respective functional homologues thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptides thereof.

In an even further aspect, the invention relates to polypeptides having cytochrome P450 enzyme activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity or even 100% sequence identity to one of the sequences SEQ ID NO: 1, SEQ ID NO: 2, SEQ ID NO: 3, SEQ ID NO: 4, SEQ ID NO: 5, SEQ ID NO: 6, and SEQ ID NO: 7 or the mature polypeptide thereof.

In a further aspect, the invention relates to a polypeptide having cytochrome B5 activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity or even 100% sequence identity to SEQ ID NO: 8, or the mature polypeptide thereof.

The invention also relates to polynucleotide sequences or genes encoding polypeptides having cytochrome P450 enzyme activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity or even 100% sequence identity to one of the sequences SEQ ID NO: 1, SEQ ID NO: 2, SEQ ID NO: 3, SEQ ID NO: 4, SEQ ID NO: 5, SEQ ID NO: 6, and SEQ ID NO: 7 or the mature polypeptide thereof, or encoding a polypeptide having cytochrome B5 activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity or even 100% sequence identity to SEQ ID NO: 8, or the mature polypeptide thereof.

In preferred embodiments, one or more of the first, second, third, fourth, fifth and sixth enzymes having cytochrome P450 activity comprises or consists of an amino acid sequence according to any one of SEQ ID NO: 1 (TwCYP82D274v1), SEQ ID NO: 2 (TwCYP82D274v2), SEQ ID NO: 74 (TwCYP82D274v3), SEQ ID NO: 75 (TwCYP82D274v4), SEQ ID NO: 3 (TwCYP71BE85), SEQ ID NO: 4 (TwCYP71BE86), SEQ ID NO: 5 (TwCYP82D213v1) and SEQ ID NO: 76 (TwCYP82D213v2), and respective functional homologs thereof having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, preferably at least 98% sequence identity thereto, or the mature polypeptide thereof.

In some embodiments, the first heterologous nucleic acid encoding a first enzyme having cytochrome P450 activity encodes TwCYP82D274 as set forth in SEQ ID NO: 1 (TwCYP82D274v1, SEQ ID NO: 2 (TwCYP82D274v2), SEQ ID NO: 74 (TwCYP82D274v3), SEQ ID NO: 75 (TwCYP82D274v4), or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the second heterologous nucleic acid encoding a second enzyme having cytochrome P450 activity encodes the cytochrome P450 enzyme TwCYP71BE86 as set forth in SEQ ID NO: 4, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the third heterologous nucleic acid encoding a third enzyme having cytochrome P450 activity encodes the cytochrome P450 enzyme

TwCYP71BE85 as set forth in SEQ ID NO: 3, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the fourth heterologous nucleic acid encoding a fourth enzyme having cytochrome P450 activity encodes the cytochrome P450 enzyme TwCYP82D213v1 as set forth in SEQ ID NO: 5 or TwCYP82D213v2 as set forth in SEQ ID NO: 76 (TwCYP82D213v2), or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the fifth heterologous nucleic acid encoding a fifth enzyme having cytochrome P450 activity encodes the cytochrome P450 enzyme TwCYP82D217 as set forth in SEQ ID NO: 6, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the sixth heterologous nucleic acid encoding a sixth enzyme having cytochrome P450 activity encodes the cytochrome P450 enzyme TwCYP82D275 as set forth in SEQ ID NO: 7, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the seventh heterologous nucleic acid encoding an enzyme having cytochrome B5 activity encodes the cytochrome B5 enzyme TwB5 #1 as set forth in SEQ ID NO: 8, or a functional homologue thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments is provided a recombinant host cell
i. wherein the host cell is capable of producing miltiradiene and/or dehydroabietadiene; and
ii. comprises a heterologous nucleic acid encoding TwCYP82D274 of SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
   wherein said cell is capable of producing 14-hydroxydehydroabietadiene.

In some embodiments is provided a recombinant host cell
i. wherein the host cell is capable of producing miltiradiene and/or dehydroabietadiene; and
ii. comprises a heterologous nucleic acid encoding TwCYP82D274 of SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof; and
iii. comprises a heterologous nucleic acid encoding TwCYP71BE86 of SEQ ID NO: 4 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
   wherein said cell is capable of producing 14-hydroxydehydroabietadiene, 3,14-dihydroxydehydroabietadiene, 3,14-dihydroxyabeodiene and/or 14-hydroxy-18-aldo-abeodiene.

In some embodiments is provided a recombinant host cell
i. wherein the host cell is capable of producing miltiradiene and/or dehydroabietadiene; and
ii. comprises a heterologous nucleic acid encoding TwCYP82D274 SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
iii. comprises a heterologous nucleic acid encoding TwCYP71BE86 of SEQ ID NO: 4 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof; and
iv. comprises a heterologous nucleic acid encoding TwCYP71BE85 of SEQ ID NO: 3 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
   wherein said cell is capable of producing 14-hydroxydehydroabietadiene, 3,14-dihydroxydehydroabietadiene, 3,14-dihydroxyabeodiene, 14-hydroxy-18-aldo-abeodiene and/or triptophenolide.

In some embodiments is provided a recombinant host cell
i. wherein the host cell is capable of producing miltiradiene and/or dehydroabietadiene; and
ii. comprises a heterologous nucleic acid encoding TwCYP82D274 of SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
iii. comprises a heterologous nucleic acid encoding TwCYP71BE86 of SEQ ID NO: 4 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
iv. comprises a heterologous nucleic acid encoding TwCYP71BE85 of SEQ ID NO: 3 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof; and
v. comprises a heterologous nucleic acid encoding TwCYP82D213 of SEQ ID NO: 5 or SEQ ID NO: 76, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
   wherein said cell is capable of producing 14-hydroxydehydroabietadiene, 3,14-dihydroxydehydroabietadiene, 3,14-dihydroxyabeodiene, 14-hydroxy-18-aldo-abeodiene, triptophenolide and/or triptonide.

In some embodiments is provided a recombinant host cell
i. wherein the host cell is capable of producing miltiradiene and/or dehydroabietadiene; and
ii. comprises a heterologous nucleic acid encoding TwCYP82D274 of SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
iii. comprises a heterologous nucleic acid encoding TwCYP71BE86 of SEQ ID NO: 4 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
iv. comprises a heterologous nucleic acid encoding TwCYP71BE85 of SEQ ID NO: 3 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
v. comprises a heterologous nucleic acid encoding TwCYP82D213 of SEQ ID NO: 5 or SEQ ID NO: 76, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof; and
vi. comprises a heterologous nucleic acid encoding TwB5 #1 of SEQ ID NO: 8 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
   wherein said cell is capable of producing triptonide with a titer that is at least 2-fold, such as at least 3-fold, such as at least 4-fold, such as at least 5-fold higher than an identical yeast cell, except wherein said yeast said does not express said TwB5 #1 or said functional homolog thereof.

In some embodiments is provided a recombinant host cell
i. wherein the host cell is capable of producing miltiradiene and/or dehydroabietadiene; and
   ii. comprises a heterologous nucleic acid encoding TwCYP82D274 of SEQ ID NO: 1, SEQ ID NO: 2, SEQ ID NO: 74 or SEQ ID NO: 75, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
   iii. comprises a heterologous nucleic acid encoding TwCYP71BE86 of SEQ ID NO: 4 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
   iv. comprises a heterologous nucleic acid encoding TwCYP71BE85 of SEQ ID NO: 3 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof;
   v. comprises a heterologous nucleic acid encoding TwCYP82D213 of SEQ ID NO: 5 or SEQ ID NO: 76, or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof; and
   vi. comprises a heterologous nucleic acid encoding TwB5 #1 of SEQ ID NO: 8 or a functional homolog thereof having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto, or the mature polypeptide thereof,
wherein said cell is capable of growing in a fermentation medium and where said fermentation medium after 7 days of fermentation comprises:
   at least 3 ppm triptonide and/or
   at least 1 ppm triptophenolide.

Aforementioned recombinant host cells may be capable of producing miltiradiene and/or dehydroabietadiene for several different reasons. For example, the host cells may endogenously be capable of producing miltiradiene. Alternatively, the recombinant host cell may comprise one or more heterologous nucleic acid sequences encoding one or more enzymes involved in the production of miltiradiene, such as the diterpene biosynthetic enzymes SPGGPPS7 of SEQ ID NO: 73 or SEQ ID NO: 81, CfTPS1 of SEQ ID NO: 67, CftTPS1 of SEQ ID NO: 77, CfTPS3 of SEQ ID NO: 68, CftTPS3 of SEQ ID NO: 78 and/or TwCPR1 of SEQ ID NO: 9, or respective functional homologs thereof having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, preferably at least 98% sequence identity thereto, or the mature polypeptides thereof.

Functional homologues of the first (e.g. TwCYP82D274), second (e.g. TwCYP71BE86), third (e.g. TwCYP71BE85), fourth (e.g. TwCYP82D213), fifth (e.g. TwCYP82D217) and sixth (e.g. TwCYP82D275) enzymes having cytochrome P450 activity and the enzyme having cytochrome B5 activity (e.g. TwB5 #1) may be verified by expressing the relevant protein in a yeast cell and assessing whether they are able to produce specific compounds as described herein below.

A yeast cell expressing a functional homolog of TwCYP82D274 (SEQ ID NO: 1, SEQ ID NO: 2, SEQ ID NO: 74 or SEQ ID NO: 75) and further expressing
i. the diterpene biosynthetic enzymes SPGGPPS7v2 (SEQ ID NO: 81), CftTPS1 (SEQ ID NO: 77), CftTPS3 (SEQ ID NO: 78) and TwCPR1 (SEQ ID NO: 9), is preferably capable of producing 14-hydroxydehydroabietadiene.

A yeast cell expressing a functional homolog of TwCYP71BE86 (SEQ ID NO: 4) and further expressing
i. the diterpene biosynthetic enzymes SPGGPPS7v2 (SEQ ID NO: 81), CftTPS1 (SEQ ID NO: 77), CftTPS3 (SEQ ID NO: 78) and TwCPR1 (SEQ ID NO: 9); and
ii. TwCYP82D274 (SEQ ID NO: 1 or SEQ ID NO: 2),
   is preferably capable of producing 14-hydroxydehydroabietadiene, 3,14-dihydroxydehydroabietadiene, 3,14-dihydroxyabeodiene and 14-hydroxy-18-aldo-abeodiene.

A yeast cell expressing a functional homolog of TwCYP71BE85 (SEQ ID NO: 3) and further expressing
i. the diterpene biosynthetic enzymes SPGGPPS7v2 (SEQ ID NO: 81), CftTPS1 (SEQ ID NO: 77), CftTPS3 (SEQ ID NO: 78) and TwCPR1 (SEQ ID NO: 9);
ii. TwCYP82D274 (SEQ ID NO: 1 or SEQ ID NO: 2); and
iii. TwCYP71BE86 (SEQ ID NO: 4),
   is preferably capable of producing 14-hydroxydehydroabietadiene, 3,14-dihydroxydehydroabietadiene, 3,14-dihydroxyabeodiene, 14-hydroxy-18-aldo-abeodiene and triptophenolide.

A yeast cell expressing a functional homolog of TwCYP82D213 (SEQ ID NO: 5 or SEQ ID NO: 76) and further expressing
i. the diterpene biosynthetic enzymes SPGGPPS7v2 (SEQ ID NO: 81), CftTPS1 (SEQ ID NO: 77), CftTPS3 (SEQ ID NO: 78) and TwCPR1 (SEQ ID NO: 9);
ii. TwCYP82D274 (SEQ ID NO: 1 or SEQ ID NO: 2);
iii. TwCYP71BE86 (SEQ ID NO: 4); and
iv. TwCYP71BE85 (SEQ ID NO: 3),
   is preferably capable of producing 14-hydroxydehydroabietadiene, 3,14-dihydroxydehydroabietadiene, 3,14-dihydroxyabeodiene, 14-hydroxy-18-aldo-abeodiene, triptophenolide and triptonide.

A yeast cell expressing a functional homolog of TwB5 #1 (SEQ ID NO: 8) and further expressing
i. the diterpene biosynthetic enzymes SPGGPPS7v2 (SEQ ID NO: 81), CftTPS1 (SEQ ID NO: 77), CftTPS3 (SEQ ID NO: 78) and TwCPR1 (SEQ ID NO: 9);
ii. TwCYP82D274 (SEQ ID NO: 1 or SEQ ID NO: 2);
iii. TwCYP71BE86 (SEQ ID NO: 4);
iv. TwCYP71BE85 (SEQ ID NO: 3); and
v. TwCYP82D213 (SEQ ID NO: 5 or SEQ ID NO: 76)
   is preferably capable of producing triptonide with a titer that is at least 2-fold, such as at least 3-fold, such as at least 4-fold, such as at least 5-fold higher than an identical yeast cell, except wherein said yeast said does not express said functional homolog of TwB5 #1.

In preferred embodiments, the enzyme having cytochrome B5 activity comprises or consists of an amino acid sequence according to SEQ ID NO: 8 (TwB5 #1), or a functional homolog thereof having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, preferably at least 98% sequence identity thereto, or the mature polypeptide thereof.

The polynucleotide of the invention may be provided by cloning from organisms that naturally produce the polypeptides such as the plant T. wilfordii or closely related plants, or it may be provided by chemical synthesis of the polynucleotide sequence based on techniques known in the art. The polynucleotide may have a sequence that is identical to a sequence found in nature, or it may have a sequence that is not found in nature, e.g. the sequence may be codon optimized for the particular selected host cell.

The polypeptides of the invention may be provided from organisms that naturally produce the polypeptides such as the plant T. wilfordii or related organisms; or they may be provided by inserting and expressing polynucleotides encoding polypeptides into a suitable host cell and recovering the polypeptides from culture broth comprising the host cell transformed with the respective genes. It is preferred to provide the polypeptides of the invention from a suitable selected recombinant host cell.

In order to transform and express a gene in a suitable host cell, the gene is usually operably connected with suitable regulatory elements and inserted into an expression vector suitable for the particular selected host cell. Selecting suitable regulatory elements, constructing a suitable expression vector and transforming the selected host cell is within the skills of the average practitioner and the invention is not limited by any particular selection of such elements.

The generated host cells comprising the genes of the invention are suitable grown in a container, e.g. a fermenter or shake flasks; under conditions where the genes are expressed and the oxygenated diterpenoid compounds are formed. When growth ceases or a sufficiently high amount of the oxygenated diterpenoid compounds are accumulated in the culture broth, the oxygenated diterpenoid compounds may be further modified and recovered from the culture broth.

Sequence identity is understood as a measurement of the similarity between two amino acid or nucleotide sequences. Sequence identity is calculated by first aligning the two sequences, counting the number of positions where the two sequences contain the same amino acid residue or nucleotide and calculating the percent identity as the number of positions with identical amino acid residue or nucleotide with the whole length of the alignment.

Several algorithms have been developed and are available for the skilled person. In this specification and claim, the sequence identity for amino acid sequences are calculated using the NCBI BLAST+pairwise alignment algorithm, using default parameters (BLOSUM 62 matrix, Gap open penalty 11; gap extend penalty 1, Exp. Thr 10), and the sequence identity for nucleotide sequences are calculated using the NCBI BLAST+pairwise alignment algorithm, using default parameters (Match/mismatch scores 1, -3; gap open penalty 5; gap extend penalty 2; exp. Thr 10). The NCBI BLAST+programs are further described in: Madeira F el at (2019) NAR 47: W636-W641.

### EXAMPLES

### Materials and Methods

Genetic Engineering of Nicotiana benthamiana

Tripterygium wilfordii CYP genes were cloned from plant material and co-expressed in Nicotiana benthamiana with the diterpene biosynthesis genes CfDXS (SEQ ID NO: 79) or SctHMGR (SEQ ID NO: 80), CfGGPPS or SpGGPPS7 (SEQ ID NO: 81), CfTPS1 (SEQ ID NO: 67) or CftTPS1 (SEQ ID NO: 77), and CfTPS3 (SEQ ID NO: 68) or CftTPS3 (SEQ ID NO: 78) using constructs and methods previously described in (1-4). Also coexpressed, were the suppressor of gene silencing, p19. Briefly, binary vectors each containing individual diterpene biosynthesis genes or Tripterygium wilfordii CYPs (TwCYPs) were transformed into agrobacteria. Liquid cultures of agrobacteria each containing specific plasmids were mixed for co-expression of specific combinations of TwCYPs.

Genetic Engineering of Saccharomyces cerevisiae and Growth Conditions for Engineered S. cerevisiae Media

YPD media: 20 g/L Bacto™ Peptone, 10 g/L Bacto™ Yeast extract, 20 g/L glucose. Synthetic complete (SC) meda without uracil: 1.92 g/L Yeast Synthetic Drop-out Media Supplements without uracil (Sigma-Aldrich Co. LLC. Catalog number Y1501), 6.7 g/L

Yeast Nitrogen Base Without Amino Acids (Sigma-Aldrich Co. LLC. Catalog number Y0626), 20 g/L glucose. Feed-In-Time (FIT) was based on EnPump200 (Enpresso GmbH), and made according to protocol enclosed with the product. Agar plates: SC media including agar (15 g/L).

Uracil auxotrophy in parent strains was introduced by selecting for lack of URA3 function on agar plates of SC medium without uracil containing also 5-Fluoroorotic Acid (5-FOA, 0.74 g/L) and uracil (30 mg/L).

Yeast transformants were isolated on SC without uracil agar plates.

Feed-Batch Fermentation of Engineering S. cerevisiae Strains for Isolation of Miltiradiene Derived Diterpenoids

All engineered S. cerevisiae strains were cultivated in 96-deepwell plates using a Feed-In-Time (FIT; m2p-labs) approach similar to previously described (insert ref Forman et al. 2018). For isolation and purification of key intermediates in the triptonide pathway selected engineered S. cerevisiae strain were cultivated in feed batch fermentor using a 2 L Biostat® A bioreactor (Sartorius AG). Fed batch fermentation was initiated by addition of a 100 mL starter culture to the reactor tank (with impellers), which in turn was prepared earlier by autoclavation while containing 200 mL Batch glucose and 300 mL Batch salt mix. Also 5 mL vitamin mix, 5 mL micro elements and 0.5 mL trace elements, were added. Cultivation in the bioreactor was started under the following conditions (monitored and automatically controlled): pH=5, temp.=30° C., dissolved oxygen (DO)=20%. While pH was controlled by feeding of ammonium hydroxide (32%) and sulfuric acid (10%), dissolved oxygen was controlled by air supply combined with stirring. Also foam levels were adjusted by addition of anti-foam emulsion (35119, Serva Electrophoresis GmbH). After 18 hours of initial cultivation in the bioreactor, feeding with Feeding solution at a rate of 1.3% was started. The fermentation process continued for 7 days with daily sampling of the culture.

Extraction of Engineered S. cerevisiae for LC-MS Analysis

Genetically engineered S. cerevisiae strain was transferred into 0.5 mL media in a 96-well plate and grown for 3 days at 30° C. with orbital shaking at 350 rpm. For extraction 0.1 mL of S. cerevisiae culture was transferred to 1.5 mL glass vials. 0.4 mL MeOH uHPLC grade was added. S. cerevisiae extract was filtered by using a 0:22 um 96-well filter plate (Merck Millipore, Darmstadt, Germany) and at stored at 4° C. prior to LC-MS analysis.

### Extraction of Diterpenoid Metabolites for LC-MS Analysis

Samples of yeast cultures for LCMS analysis were prepared in 1.5 mL glass vials by mixing yeast cultures and methanol spiked with 5 ppm andrographolide (internal standard; FA17902, CarboSynth) in a ratio of 1:19 (v/v) for daily bioreactor samples and 1:4 (v/v) for 96-deepwell cultures. Mixing proceeded for 30 min with shaking at room temp. For tobacco samples, 2 leaf discs (ø=3 cm) placed in 1.5 ml glass vials, were extracted with 1 mL of the methanol extraction solution, for 1 h with shaking at room temperature. Before LCMS analysis samples were passed through a 0.22 μM 96-well plate filter (Merck Millipore, Darmstadt, Germany) and stored at 5° C.

### LC-MS Analysis:

Methanol (MeOH) extracts were analysed using an Ultimate 3000 UHPLC+Focused system (Dionex Corporation, Sunnyvale, CA) coupled to a Bruker Compact ESI-QTOFMS (Bruker) system. Samples were separated on a Kinetex XB-C18 column (100×2.1 mm ID, 1:7 μm particle size, 100° A pore size; Phenomenex Inc., Torrance, CA) maintained at 40_C with a flow rate of 0.3 mL min-1 and mobile phase consisting of 0.05% (v/v) formic acid in water (solvent A) and 0.05% (v/v) formic acid in acetonitrile (solvent B).

Two LC Protocols were Used:

LC method 1:0-0.5 min, 10% B; 0.5-21 min, linear increase from 10 to 80% B; 21-31 min, to 90% B; 31-34 min, to 100% B; 34-39 min 100% B; 39-40 min linear decrease from 100 to 10% B.

LC method 2:0-0.5 min, 20% B; 0.5-11 min, linear increase from 20 to 80% B; 11-20 min, to 90% B; 20-22 min, to 100% B; 22-27 min 100% B; 27-28 min linear decrease from 100 to 20% B.

LC method 3:0-0.5 min, 20% B; 0.5-9 min, linear increase from 20 to 100% B; 9-11 min, 100% B; 11-11.5 min, linear decrease from 100 to 20% B; 11.5-15 min, 20% B.

### Extraction of Diterpenoid Metabolites for GC-MS Analysis

Samples of yeast cultures for GCMS analysis were prepared in 1.5 mL glass vials by mixing yeast culture and pure methanol at a ratio of 1:4 (v/v). After brief mixing, apolar constituents, were liquid-liquid extracted into hexane, spiked with 10 ppm 1-eicocene, by mixing at a ratio of 1:1 (v/v) and shaking for 1 h. For tobacco samples, 2 leaf discs (ø=3 cm) placed in 1.5 ml glass vials, were extracted with 1 mL of the same hexane solution, via 1 h of shaking. Prior to GCMS analysis hexane layers were transferred to new vials.

### Gas Chromatography Mass Spectrometry (GC-MS) Analysis

GC-MS analysis was carried out on a Shimadzu GCMS-QP2010 Ultra (Shimadzu Corp.) with an Agilent HP-5 MS column (Agilent Technologies) 20 m×0.18 mm i.d., 0.18 μm film thickness). Hydrogen was used as a carrier gas at a constant linear velocity of 50 cm s-1, and the injection volume was 1 μL at 250° C. (splitless mode). The oven program was 80° C. for 2 min, ramp at rate 20° C./min to 180° C., ramp at rate 10° C./min to 300° C., ramp at rate 20° C./min to 310° C., hold for 3 min. Data was stored in .CDF format and processed in MZmine2.

### Relative Quantification of Miltiradiene Derived Diterpenoids

Relative compound quantities in yeast cultures were based on normalized peak areas of characteristic ions (data obtained using targeted feature detection in the MZmine2 software). The signal for the following ions were quantified: 1: miltiradiene m/z 91.1, 2:14-hydroxyabietadiene m/z 189.1, 3: F15P1 m/z 303.2318, 4: F20P2 m/z 283.2059, 5: F15P2 m/z 299.2002, 6: triptophenolide m/z 313.1794, 8: triptonide m/z 359.1481. For LCMS and GCMS data a mass deviation of 5 ppm and 100 ppm, respectively, was tolerated.

The peak area of the base peak ion (m/z 315.1947) for the internal standard andrographolide was used for normalization.

### Absolute Quantifications

Absolute quantifications of triptophenolide (FT65732, CarboSynth) and triptonide (FT65197, CarboSynth) were done by co-analysis of authentic standards prepared in methanol and a final concentration of 5 ppm internal standard (andrographelide). Quantification was based on normalized peak area and calculated from the slopes of linear extrapolations of the standards response curve (triptophenolide 0.05, 0.5, 1, 2 ppm; triptonide 0.5, 1, 2, 10, 20 ppm).

Isolation and Purification of Miltiradiene Derived Compounds from Engineered S. Cerevisiae Strain for NMR Analysis

Compounds in this invention were isolated from bioreactor cultures yeast strains NVJ8.15, and NVJ3.10, and structurally elucidated by NMR. The combined ethyl acetate extracts of broth and methanol-lysed cells (cells: methanol=1:4, v/v) were initially dried in presence of Celite SR (06858, Sigma-Aldrich) via rotary evaporation. Compounds were subsequently isolated by successive fractionations using a puriFlash® 5.250 (Interchim, Montluçon, France) instrument with detection by UV absorbance and Evaporative Light-Scattering Detection (ELSD). This was equipped with either of columns (C1) PF-15SIHP-F0025 (OV002A, Interchim) and (C2) US5C18HQ-100/300 (SSP750, Interchim) for normal phase- and reverse phase separation, respectively.

An initial pre-fractionation of the dry mix of Celite S®/crude extract was achieved using column (ref. 9) with loading from a manually packed dry-loading column. Separation was obtained using mobile phases hexane (A) and ethyl acetate (B), a constant flow rate of 15 mL/min, followed by a final washing step with 100% methanol. Compounds of interest were detected by UV and ELSD and collected. Collected fractions were continuously evaluated by LCMS using LC-MS method 3 and TLC analysis prior to further fractionation or NMR studies. Additional purification of compounds of interest from fraction with multiple compounds was done by an additional normal phase fraction using C1 or a reverse phase column fractionation using C2.

For reverse phase purification with C2 samples were evaporated using rotor evaporation and resuspended in 2 mL methanol. Sample was injected directly onto the pre conditioned column C2. Mobile phases for C2 consisted of solvent C: deionized water and solvent D acetonitrile each acidified with 0.05% (v/v) formic acid. A constant flow rate of 32 mL/min, was used, with a linear solvent gradient with increasing concentration of solvent D. Compounds of interest were detected by ELSD and UV and collected.

Additional reverse phase purification was done by multiple injections of 100 μL ontp a semi-prep Phenomenex Luna 5 μm C18 (2) 100 Å 250×10 mm (fully porous) (Phenomenex, Inc., Torrance, CA, USA) column on a Shimadzu HPLC (SPD-M20A diode array detector, FRC-10A fraction collector, DGU-20A5 degasser, LC-20AT pump, CBM-20A System controller, CTO-10AS VP column oven, SIL-10AP autosampler). Mobile phase was a linear gradient between C and D with an increasing amount of D going from 50-100%. Compounds of interest was detected by UV absorbance at 210 nm and collected.

### Mass Spectra

Mass spectra were acquired in positive ion mode over a scan range of m/z 50-1200 with the following ESI and MS settings: capillary voltage, 4000 V; end plate offset, 500 V; dry gas temperature, 220° C.; dry gas flow of 8 L min1; nebulizer pressure, 2 bar; in source CID energy, 0 eV; hexapole RF, 50 Vpp; quadrupole ion energy, 4 eV; collision cell energy, 7 eV. Raw chromatogram data was calibrated using an internal sodium formate standard and subsequently exported as mzML format using DataAnalysis 4.3 (Build 110.102.1532) (64-bit), Bruker. MZmine ver 2.53 was used for visualizing the LC-MS chromatograms.

### Media Recipes for Bioreactor Starting Media and Feed Media

### Batch Glucose:

Glucose monohydrate 55 g/L

### Batch Salt Mix:

Ammonium sulfate 25 g/L
Potassium phosphate monobasic 5 g/L
Magnesium sulfate heptahydrate 1.7 g/L

### Feed Glucose:

Glucose monohydrate 880 g/L

### Feed Salt Mix:

Potassium phosphate monobasic 21.6 g/L
Magnesium sulfate heptahydrate 24.24 g/L
Potassium sulfate 8.4 g/L
Sodium sulfate 0.672 g/L

### Preparation Notes:

Batch- and feed salt mixes as well as batch and feed glucose were prepared in separate BlueCap bottles by dissolving components in Milli-Q water and sterilizing by autoclavation.

### Feeding Solution:

A feeding solution was made by mixing 500 mL of feed glucose with 500 ml of feed salt mix, 10 mL of vitamin mix, 10 mL of micro elements solution and 1 mL of trace elements solution.

### Example 1: Expression in Nicotiana benthamiana

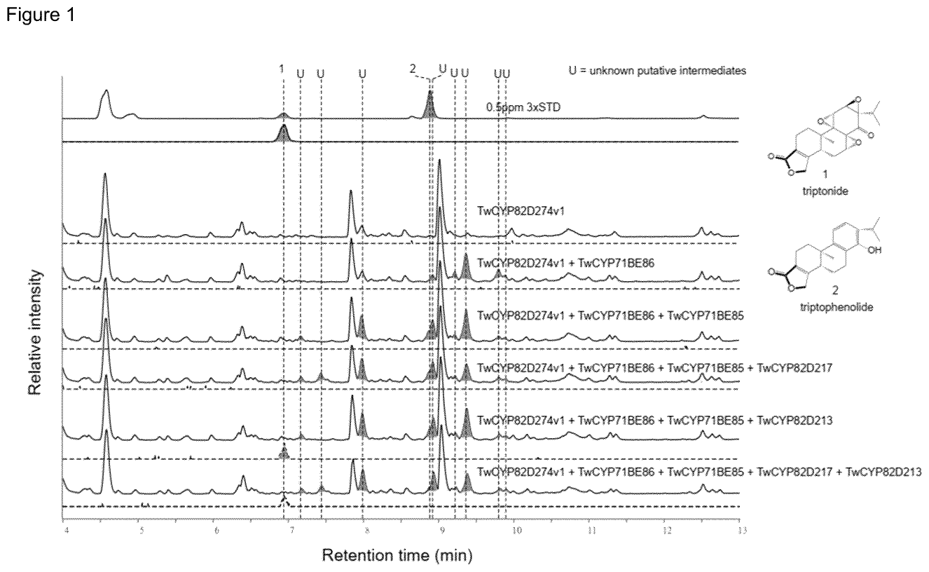

Leaf material of N. benthamiana co-expressing specific combinations of genes of interest (GOI) was harvested 7 days after agrobacterial infiltration. 1 mL methanol (MeOH) was added to 2 leaf disks (Ø=2 cm). Extraction was done at room temperature at 200 rpm orbital shaking. 200 uL of extract was filtered by using a 0:22 μm 96-well filter plate (Merck Millipore, Darmstadt, Germany) and at stored at 4° C. prior to LC-MS analysis. FIG. 1 shows the obtained LCMS profiles. The results show that the N. benthamiana cells transformed with CYP82D274V1 encoding the enzyme having SEQ ID NO: 1, leads to production of 14-OH-dehydroabietadiene; when the cells are further transformed with CYP71BE85 and CYP71BE86 encoding the enzymes having the amino acid sequences SEQ ID NO: 3 and SEQ ID NO: 4, respectively, triptophenolide is formed; and when the cells are further transformed with CYP82D213 encoding the enzyme having the amino acid sequence of SEQ ID NO: 5 triptonide is formed. Further, it can be seen that the enzyme having the sequence of SEQ ID NO:6 and encoded by the gene CYP82D217 increases the production of triptophenolide and triptonide.

### Example 2: Construction of S. cerevisiae Strains

### Strain Construction

Parent yeast strain was S. cerevisiae S288C (NCYC 3608; National Collection of Yeast Cultures, Norwich, UK).

Genotypes and source of strains are listed in table 3.

Constructed yeast strains were made using the lithium acetate transformation method (8). Parent strains without functional URA3 were made competent by the following procedure: Inoculation from a glycerol stock into 5 ml YPD medium and growing at 30° C. O/N. Then, transfer of 3 mL of O/N culture to 50 mL YPD medium and continued growing for 4-5 hours followed by centrifugation at 4000 RPM for 10 minutes then discarding the supernatant. Cells were then ready for transformation after 2 washes in sterile water (1st in 25 mL, 2nd in 1 mL) and resuspension in 0.4 mL of sterile water.

Transformation of competent yeast cells was done by the following procedure: Mixes of designated Notl digested plasmids (2 μL of each) were each added 10 μL competent yeast cells and mixed with 60 μL PEG 3350 (50% w/v), 9 μL LiAc (1 M) and 12.5 μL preboiled salmon sperm DNA (10 mg/ml). The resulting mixes were next incubated at 42° C. for 40 minutes before cells were collected by centrifugation (3000 RPM for 5 minutes) and removal of supernatant. Cells were then resuspended in 100 μL sterile water and spread on SC without uracil agar plates. Isolated transformants appeared as single colonies after 2 days of incubation at 30° C. Insertion of gene constructs was confirmed by colony PCR, using gene and construct specific primers found in table 1. For colony PCR, yeast colonies were resuspended in 50 μL 20 mM NaOH and incubated at 99° C. for 15 min. 1 μL colony suspension was used for PCR.

TABLE 1
List of primers used

| | | Number | | Target |
|---|---|---|---|---|
| | | in | | vector (Entry |
| | | Sequence | Target | and/or |
| Name | Sequence | listing | gene | Destination) |
| CO_TwCYP71BE85v1_TEF-F | AGCGATACGNAAAATGGACTTATTGCAATTTCCA | 10 | CO_TwCYP7 | pX-3-Ass1- |
| | TCTG | | 1BE85v1 | KIURA3 |
| CO_TwCYP71BE85v1_TEF-R | CACGCGANTCAGTTAAATGCGGGTGATGG | 11 | | |
| CO_TwGA3OX1_TEF-F | AGCGATACGNAAAATGAGTCCTCCGCCTACAATA | 12 | CO_TwGA3 | pX-3-Ass3 |
| | | | OX1 | |
| CO_TwGA3OX1_TEF-R | CACGCGANTTAAATACCTAAAAGCGAGACGGG | 13 | | |
| CO_AcoUGT2_TEF-F | AGCGATACGNAAAATGGCTGTTAGCTTAAAAAAT | 14 | CO_AcoUGT | pX-3-Ass3 |
| | ACCG | | 2 | |
| CO_AcoUGT2_TEF-R | CACGCGANTTAACGACTGATATGAGCGACG | 15 | | |
| CO_TwCYP82D213_PGK-F | ATCAACGGGNAAAATGGAATTCCTTCTGTCA | 16 | CO_TwCYP8 | pX-3-Ass3 |
| | TTGC | | 2D213 | |
| CO_TwCYP82D213_PGK-R | CGTGCGANCTAACCCATGTAAAGATGTGATGG | 17 | | |
| CO_TwCYP71BE86_PGK-F | ATCAACGGGNAAAATGGACTTACAATTACCTAGC | 18 | CO_TwCYP7 | pX-3-Ass1- |
| | TTCC | | 1BE86 | KIURA3 |
| CO_TwCYP71BE86_PGK-R | CGTGCGANTTAACCAGATAAACTACGATATGGG | 19 | | |
| TwCYP82D217_pLife-F | GGCTTAANAAGCATCTTCTCTCCTAACTAGCTTT | 20 | TwCYP82D2 | pLife |
| | CTAAAT | | 17 | |
| TwCYP82D217_pLife-R | GGTTTAANCTATTGCAATTCACCCCATGTAGACA | 21 | | |
| | A | | | |
| pLifeUP_TEF-F | AGCGATACGNGACCTGCAGGCTGAGGCTT | 22 | TwCYP82D2 | pAss2 |
| | | | 17 | |
| pLife_TEF-R | CACGCGANCCCGGGGCTGAGGTTTAAT | 23 | | |
| TwCYP82D274v1_pLife-F | GGCTTAANATGGAGTTTCTTCTTTCACTCCCAAC | 24 | TwCYP82D2 | pLife |
| | A | | 74v1 | |
| TwCYP82D274v1_pLife-R | GGTTTAANTCAGCCCATATAGAGATGAGCTGGGA | 25 | | |
| | G | | | |
| pLife_TEF-F | AGCGATACGNTGCAGGCTGAGGCTTAATATG | 26 | TwCYP82D2 | pX-4-SI- |
| | | | 74v1 | KIURA3 |
| pLife_TEF-R | CACGCGANCCCGGGGCTGAGGTTTAAT | 27 | | |
| TwCPR1_pLife-F | GGCTTAANATGCAATCTTCTTCAAATTCTATGAA | 28 | TwCPR1 | pLife |
| | GG | | | |
| TwCPR1_pLife-R | GGTITAANTTACCACACATCCCGGAGATA | 29 | | |
| pLife_PGK-F | ATCAACGGGNTGCAGGCTGAGGCTTAATATG | 30 | TwCPR1 | pX-4-SI- |
| | | | | KIURA3 |
| pLife_PGK-R | CGTGCGANCCCGGGGCTGAGGTTTAAT | 31 | | |
| TwB5#1_pLife-F | GGCTTAANATGGCTTCGGATCGGAAGATA | 32 | TwB5#1 | pLife |
| TwB5#1_pLife-R | GGTTTAANCTATTCTTTCTTGGTGAAGTGACGTA | 33 | | |
| pLife_PGK-F | ATCAACGGGNTGCAGGCTGAGGCTTAATATG | 34 | TwB5#1 | pAss2 |
| pLife_PGK-R | CGTGCGANCCCGGGGCTGAGGTTTAAT | 35 | | |
| TwB5#2_pLife-F | GGCTTAANATGGGTGGAGACGGAAAGGTT | 36 | TwB5#2 | pLife |
| TwB5#2_pLife-R | GGTTTAANTTAAGCAGGAGGAGCTGATTTGGT | 37 | | |
| pLife_PGK-F | ATCAACGGGNTGCAGGCTGAGGCTTAATATG | 38 | TwB5#2 | pAss2 |
| pLife_PGK-R | CGTGCGANCCCGGGGCTGAGGTTTAAT | 39 | | |
| TwB5#3_pLife-F | GGCTTAANATGGCTGGTCAGAGAGTTTTCAC | 40 | TwB5#3 | pLife |
| TwB5#3_pLife-R | GGTTTAANTTAGAAGATCTGCTCAGGCCTTGTA | 41 | | |
| pLife_PGK-F | ATCAACGGGNTGCAGGCTGAGGCTTAATATG | 42 | TwB5#3 | pAss2 |
| pLife_PGK-R | CGTGCGANCCCGGGGCTGAGGTTTAAT | 43 | | |
| TwB5#4_PGK-F | ATCAACGGGNAAAATGGCTAAACTTCTTTCATTT | 44 | TwB5#4 | pAss2 |
| | GCTGAG | | | |
| TwB5#4_PGK-R | CGTGCGANTTAGAAAAGGTATCGCAAACCAAATG | 45 | | |
| | CC | | | |
| TwB5#5_PGK-F | ATCAACGGGNAAAATGATTATTGTTGCGGTGGCT | 46 | TwB5#5 | pAss2 |
| | CTGA | | | |
| TwB5#5_PGK-R | CGTGCGANTTACTTCTCTAGATCCCCAATGTAAA | 47 | | |
| | AATCATCG | | | |
| TwB5#6_PGK-F | ATCAACGGGXAAAATGCCGACTTTAACGAAGCTG | 48 | TwB5#6 | pAss2 |
| | CAC | | | |
| TwB5#6_PGK-R | CGTGCGAXCTACTTCTTCCGCAAGTACAGGAGTC | 49 | | |
| YEA85_UP_Genotyping_Fw | TCTCAGGTATAGCATGAGGTCGCTCAT | 50 | Genotyping | UP_ |
| | | | | Genotyping |
| YEA86_DW_Genotyping_Fw | CCTGCAGGACTAGTGCTGAGGCATTAAT | 51 | Genotyping | DW_ |
| | | | | Genotyping |
| YEA87_X-2_Genotyping_UP | GTTTGTAGTTGGCGGTGGAG | 52 | Genotyping | X-2_ |
| | | | | Genotyping |
| YEA88_X- | GAGACAAGATGGGGCAAGAC | 53 | Genotyping | X-2_ |
| 2_Genotyping_DW | | | | Genotyping |
| YEA89_X-3_Genotyping_UP | TGACGAATCGTTAGGCACAG | 54 | Genotyping | X-3_ |
| | | | | Genotyping |
| YEA90_X- | CCGTGCAATACCAAAATCGAG | 55 | Genotyping | X-3_ |
| 3_Genotyping_DW | | | | Genotyping |
| YEA91_X-4_Genotyping_UP | CTCACAAAGGGACGAATCCT | 56 | Genotyping | X-4_ |
| | | | | Genotyping |
| YEA92_X- | GACGGTACGTTGACCAGAG | 57 | Genotyping | X-4_ |
| 4_Genotyping_DW | | | | Genotyping |
| YEA93_XI-1_Genotyping_UP | CTTAATGGGTAGTGCTTGACACG | 58 | Genotyping | XI-1_ |
| | | | | Genotyping |
| YEA94_XI-2_Genotyping_UP | GTTTGTAGTTGGCGGTGGAG | 59 | Genotyping | XI-2_ |
| | | | | Genotyping |
| YEA95 X1- | GAGACAAGATGGGGCAAGAC | 60 | Genotyping | X1-2_ |
| 2_Genotyping_DW | | | | Genotyping |
| YEA96_XI-5_Genotyping_UP | CTCAATGATCAAAATCCTGAATGCA | 61 | Genotyping | XI-5_ |
| | | | | Genotyping |
| YEA97_X1- | GCATGGTCACCGCTATCAGC | 62 | Genotyping | X1-5_ |
| 5_Genotyping_DW | | | | Genotyping |
| YEA98_XII- | CGAAGAAGGCCTGCAATTC | 63 | Genotyping | XII-2_ |
| 2_Genotyping_UP | | | | Genotyping |
| YEA99_X1I- | GGCCCTGATAAGGTTGTTG | 64 | Genotyping | X1I-2_ |
| 2_Genotyping_DW | | | | Genotyping |
| YEA100_XII- | CCACCGAAGTTGATTTGCTT | 65 | Genotyping | XII-5_ |
| 5_Genotyping_UP | | | | Genotyping |
| YEA101_X1I- | GTGGGAGTAAGGGATCCTGT | 66 | Genotyping | XII-5_ |
| 5_Genotyping_DW | | | | Genotyping |

Assembly of Genetic Constructs for S. cerevisiae Genome Engineering

Plasmid names and encoded gene constructs are listed in table 2. All plasmids were generated by USER cloning as previously described (5). Also, parent vectors named assembler-1, -2 and -3, for simultaneous genome integration of up to six gene constructs, and harboring AsiSI/Nb.Bsml USER-cassettes, were prepared for USER cloning as previously described (6). Primers used for PCR amplification with USER compatible PfuX7 polymerase (7) are listed in table 1. Vectors used and generated in this work is listed in table 3.

Codon optimized genes for S. cerevisiae were acquired from TWIST Biosciences, USA, San Francisco. All genes denoted with the prefix “CO_” in the below tables were codon-optimized. Codon-optimized genes were amplified using primers identical to those described in Table 1 above, except that the primers were modified to accommodate hybridization to any nucleotide changes in the codon-optimized genes. The primers for amplification of the codon-optimized genes are also disclosed in J. Andersen-Ranberg et al., Expanding the Landscape of Diterpene Structural Diversity through Stereochemically Controlled Combinatorial Biosynthesis. Angewandte Chemie International Edition, n/a (2016).

TABLE 2
Vectors and plasmids generated and used

| Vectors for yeast genome-integration | | |
|---|---|---|
| Name | Description | Source |
| pCYT183 | pX-3-Ass1-KIURA3- | This study |
| | PpTDH3::CO_TwCYP71BE86 | |
| pCYT184 | pX-3-Ass1-KIURA3- | This study |
| | TpCCW12::CO_TwCYP71BE85v1 | |
| pCYT185 | pX-3-Ass1-KIURA3- | This study |
| | PpTDH3::CO_TwCYP71BE86- | |
| | TpCCW12::CO_TwCYP71BE85v1 | |
| pCYT186 | pX-3-Ass3-PpENO2::CO_TwCYP82D213 | This study |
| pCYT187 | pX-3-Ass3-TpPDC1::CO_TwGAOX1 | This study |
| pCYT188 | pX-3-Ass3-PpENO2::CO_TwCYP82D213- | This study |
| | TpPDC1::CO_TwGAOX1 | |
| p349 | pAss2-PpFBA1::TwB5#2- | This study |
| | TpSED1::TwCYP82D217 | |
| p350 | pAss2-PpFBA1::TwB5#3- | This study |
| | TpSED1::TwCYP82D217 | |
| p351 | pAss2-PpFBA1::TwB5#1- | This study |
| | TpSED1::TwCYP82D217 | |
| p352 | pAss2-PpFBA1::TwB5#6- | This study |
| | TpSED1::TwCYP82D217 | |
| p353 | pAss2-PpFBA1::TwB5#5- | This study |
| | TpSED1::TwCYP82D217 | |
| p354 | pAss2-PpFBA1::TwB5#4- | This study |
| | TpSED1::TwCYP82D217 | |
| p355 | pAss2-TpSED1::TwCYP82D217 | This study |
| pJAR1 | pX-3-Ass3-TpPDC1::CO_AcUGT2 | This study |
| pJAR2 | pX-3-Ass3-PpENO2::CO_TwCYP82D213- | This study |
| | TpPDC1::CO_AcUGT2 | |
| p320 | pX-4-SI-PpTEF2::TwCPR1- | This study |
| | TpTDH3::TwCYP82D274v1 | |
| pVictor1 | pXI-2-Ass1-pICL1::CO_SpGGPPS7 | This study |
| pVictor2 | pAss2-pPGK1::CO_CftTPS3 | This study |
| pVictor3 | pXI-2-Ass3-pTEF1::CO_CftTPS3 | This study |
| pCYT85 | pAss2A-PpPGK1::CO_CftTPS1- | This study |
| | TpTEF1::CO_CftTPS3 | |
| pTRIP10 | pX-3-Ass3-PpSED1::TwCYP82D274v1- | This study |
| | TpFBA1::Twb5#1 | |
| pTRIP108 | pAss2C-PpENO2::CO_TwCYP71BE85 | This study |
| pTRIP110 | pX-3-Ass3-PpSED1::TwCYP82D274v1 | This study |
| pTRIP14 | pSIXI-2-PpPGK1::TwCPR1- | This study |
| | TpTPI1::Twb5#4 | |
| pTRIP4 | pAss2B-PpCCW12::CO_TwCYP82D213- | This study |
| | TpSED1::CO_TwCYP71BE86 | |
| pTRIP5 | pAss2C-PpENO2::CO_TwCYP71BE85- | This study |
| | TpPDC1::CO_TwCYP82D213 | |
| pTRIP50 | pXII-2-Ass1-PpPGK1::CO_TwCYP71BE86- | This study |
| | TpTPI1::CO_TwCYP71BE85 | |
| pTRIP52 | pAss2A-PpTDH3::CO_TwCYP82D213- | This study |
| | TpSED1::CO_TwCYP82D213 | |
| pTRIP53 | pAss2B-PpPDC1::TwCYP82D274v1- | This study |
| | TpENO2::TwCYP82D274v1 | |
| pTRIP54 | pAss2C-PpTEF1::TwCYP82D213- | This study |
| | TpPGK1::TwCYP82D213 | |
| pTRIP55 | pXII-2-Ass3-PpSED1::Twb5#1- | This study |
| | TpFBA1::Twb5#4 | |
| pTRIP7 | pX-3-Ass1-pTEF2::TwCPR1- | This study |
| | TpICL1::CO_SpGG-PPS7 | |
| pTRIP8 | pX-3-Ass1-pTEF2::TwCPR2- | This study |
| | TpICL1::CO_SpGG-PPS7 | |
| pTRIP88 | pAss2B-TpSED1::CO_TwCYP71BE86 | This study |
| pTRIP92 | pX-3-Ass3-PpSED1::TwCYP82D274v3- | This study |
| | TpFBA1::Twb5#1 | |
| pTRIP95 | pX-3-Ass3-PpSED1::TwCYP82D274v4- | This study |
| | TpFBA1::Twb5#1 | |
| pTRIP89 | pAss2C-PpENO2::CO_TwCYP71BE85- | This study |
| | TpPDC1::CO_TwCYP82D213v2 | |
| pTRIP3 | pAss2-PpFBA1::Twb5#1 | This study |
| pCYT185 | pX-3-Ass1-PpTDH3::CO_TwCYP71BE86- | This study |
| | TpCCW12::CO_TwCYP71BE85 | |
| pX-3-Ass3 | pX-3-Ass3 empty | This study |
| pX-3-Ass1 | pX-3-Ass1 empty | This study |
| pAss2A | pAss2A empty | This study |
| pAss2B | pAss2B empty | This study |
| pAss2C | pAss2C empty | This study |
| pVic1 | pLIFE-SctHMGR | This study |
| pVic2 | pLIFE-CftTPS1 | This study |
| pVic3 | pLIFE-CftTPS3 | This study |
| pVic4 | pLIFE-SpGGPPS7 | This study |
| 239.TwCYP756A1 | pLIFE-TwCYP82D274v1 | This study |
| 59.TwCYP10 | pLIFE-TwCYP71BE85 | This study |
| 81.TwCYP9 | | |
| 81.TwCYP9 | pLIFE-TwCYP71BE86 | This study |
| 297.TwB5#1 | pLIFE-Twb5#1 | This study |
| 46.TwCYP17 | pLIFE-TwCYP82D213 | This study |
| P19 | pBin61-p19 | Voinnet et |
| | | al., 2003 |
| | | (ref.: 10) |
| pDXS | pLIFE-CfDXS | This study |
| pCfTPS1 | pLIFE-pCfTPS1 | This study |
| pCfTPS2 | pLIFE-pCfTPS2 | This study |
| pCfGGPPS | pLIFE-pCfGGPPS | This study |

TABLE 3
List of S. cerevisiae strains used and generated.

| Name | Genotype | Source |
|---|---|---|
| S288c | MATα, SUC2, gal2, mal2, mel, flo1, flo8-1, | National Collection of |
| | ho, bio1, bio6 | Yeast Cultures |
| | | (NCYC) |
| NVJ0 | MATα, SUC2, gal2, mal2, mel, flo1, flo8-1, | This study |
| | ho, bio1, bio6, ura3Δ::KanMX, XI-2::(pTEF1- | |
| | CO_CftTPS1/pPGK1-CO_CftTPS3/pICL1- | |
| | CO_SpGGPPS7/KIURA3) | |
| NVJ1-3.5 | MATα, SUC2, gal2, mal2, mel, flo1, flo8-1, | This study |
| | ho, bio1, bio6, ura3Δ::KanMX, XI-2::(pTEF1- | |
| | CO_CftTPS1/pPGK1-CO_CftTPS3/pICL1- | |
| | CO_SpGGPPS7), X-4::(pTDH3- | |
| | TwCYP82D274v1/pTEF2-TwCPR1/KIURA3) | |
| NVJ3.10 | MATα, SUC2, gal2, mal2, mel, flo1, flo8-1, | This study |
| | ho, bio1, bio6, ura3Δ::KanMX, XI-2::(pTEF1- | |
| | CO_CftTPS1/pPGK1-CO_CftTPS3/pICL1- | |
| | CO_SpGGPPS7), X-4::(pTDH3- | |
| | TwCYP82D274v1/pTEF2-TwCPR1), X- | |
| | 3::(pTDH3-CO_TwCYP71BE86/pCCW12- | |
| | CO_TwCYP71BE85v1/pFBA1-Twb5#1) | |
| NVJ2-19 | MATα, SUC2, gal2, mal2, mel, flo1, flo8-1, | This study |
| | ho, bio1, bio6, ura3Δ::KanMX, XI-2::(pTEF1- | |
| | CO_CftTPS1/pPGK1-CO_CftTPS3/pICL1- | |
| | CO_SpGGPPS7), X-4::(pTDH3- | |
| | TwCYP82D274v1/pTEF2-TwCPR1), X- | |
| | 3::(pTDH3-CO_TwCYP71BE86/pCCW12- | |
| | CO_TwCYP71BE85v1/pENO2- | |
| | CO_TwCYP82D213/pSED1- | |
| | TwCYP82D217/pFBA1-TwB5#1/KIURA3) | |
| NVJ11-0 | MATα, SUC2, gal2, mal2, mel, flo1, flo8-1, | This study |
| | ho, bio1, bio6, ura3Δ::KanMX, X-3::(KIURA3) | |
| NVJ11-1 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/KIURA3) | |
| NVJ11-2 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | TwCYP82D274V1/KIURA3) | |
| NVJ11-3 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pENO2- | |
| | CO_TwCYP71BE85v1/pSED1- | |
| | TwCYP82D274V1/KIURA3) | |
| NVJ11-4 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pSED1- | |
| | TwCYP82D274V1/KIURA3) | |
| NVJ11-5 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pSED1- | |
| | TwCYP82D274V1/KIURA3) | |
| NVJ11-6 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213/pSED1- | |
| | TwCYP82D274V1/KIURA3) | |
| NVJ11-7 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | TwCYP82D274V1/pFBA1-Twb5#1/KIURA3) | |
| NVJ11-8 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pENO2- | |
| | CO_TwCYP71BE85v1/pSED1- | |
| | TwCYP82D274V1/pFBA1-Twb5#1/KIURA3) | |
| NVJ11-9 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pSED1- | |
| | TwCYP82D274V1/pFBA1-Twb5#1/KIURA3) | |
| NVJ11-10 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pSED1- | |
| | TwCYP82D274V1/pFBA1-Twb5#1/KIURA3) | |
| NVJ11-11 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213/pSED1- | |
| | TwCYP82D274V1/pFBA1-Twb5#1/KIURA3) | |
| NVJ8-15 | MATα, SUC2, gal2, mal2, mel, flo1, flo8-1, | This study |
| | ho, bio1, bio6, ura3Δ::KanMX, X-3::(pTEF2- | |
| | TwCPR2/pICL1-CO_SpGGPPS7/pPGK1- | |
| | CO_CftTPS1/pTEF1-CO_CftTPS3/pCCW12- | |
| | CO_TwCYP82D213/pSED1- | |
| | CO_TwCYP71BE86/pENO2- | |
| | CO_TwCYP71BE85v1/pPDC1- | |
| | TwCYP82D213/pSED1-TwCYP82D274V1) | |
| | XI-2::(pPGK1-TwCPR1/pTPI1-Twb5#4) | |
| | XII-2::(pPGK1-CO_TwCYP71BE86/pTPI1- | |
| | TwCYP71BE85v1/pTDH3- | |
| | CO_TwCYP82D213/pSED1- | |
| | CO_TwCYP82D213/pPDC1- | |
| | TwCYP82D274V1/pENO2- | |
| | TwCYP82D274V1/pTEF1- | |
| | TwCYP82D213/pPGK1- | |
| | TwCYP82D213/pSED1-Twb5#1/pFBA1- | |
| | Twb5#4/KIURA3) | |
| NVJ10-1 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213/pSED1- | |
| | TwCYP82D274v1/pFBA1-Twb5#1/KIURA3) | |
| NVJ10-3 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213/pSED1- | |
| | TwCYP82D274v3/pFBA1-Twb5#1/KIURA3) | |
| NVJ10-6 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213/pSED1- | |
| | TwCYP82D274v4/pFBA1-Twb5#1/KIURA3) | |
| NVJ10-8 | MATα, SUC2, gal2, mal2, mel, flo1, flo8-1, | This study |
| | ho, bio1, bio6, ura3Δ::KanMX, X-3::(pTEF2- | |
| | TwCPR1/pICL1-CO_SpGGPPS7/pPGK1- | |
| | CO_CftTPS1/pTEF1-CO_CftTPS3/pSED1- | |
| | CO_TwCYP71BE86/pENO2- | |
| | CO_TwCYP71BE85v1/pPDC1- | |
| | CO_TwCYP82D213v2/pSED1- | |
| | TwCYP82D274V1/pFBA1-Twb5#1/KIURA3) | |

### Example 3: Expression in the Yeast, Saccharomyces cerevisiae

### Extraction and Metabolite Analysis

Genetically engineered S. cerevisiae strains were transferred into 0.5 mL media in a 96-well plate and grown for 3 days at 30° C. with orbital shaking at 350 rpm. For extraction 0.1 mL of S. cerevisiae culture was transferred to 1.5 mL glass vials. 0.4 mL MetOH uHPLC grade was added. S. cerevisiae extracts extract was filtered by using a 0:22 μm 96-well filter plate (Merck Millipore, Darmstadt, Germany) and at stored at 4° C. prior to LC-MS analysis.

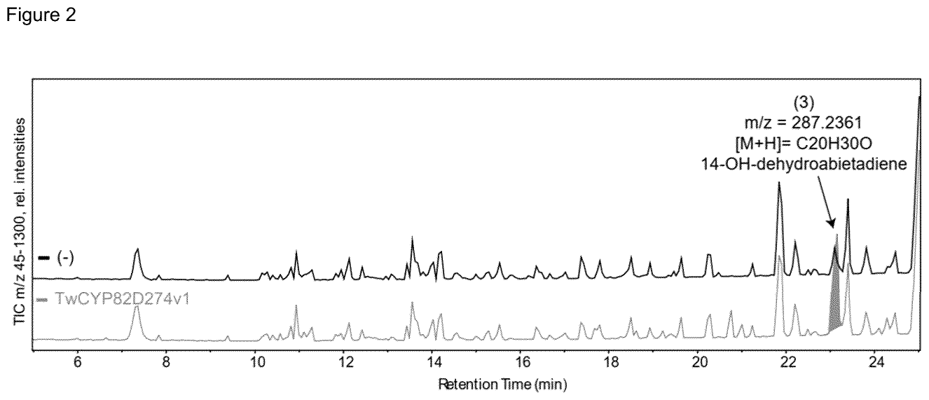

LCMS profiles of the extracts can be seen in FIG. 2, where it can be observed that transforming the background strain with TwCYP82D274V1 encoding the enzyme having the amino acid sequence of SEQ ID NO: 1 leads to formation of 14-OH-dehydroabietadiene.

### Example 4: Detection of 14-OH-Dehydroabietadiene by NMR Analysis

The compound identified as 14-OH-dehydroabietadiene in example 3 was analyzed by NMR to confirm its identity.

### Purification for NMR

Purification of Triptolide Intermediates from Engineered Yeast

Engineered yeast producing the desired compound of interest was inoculated from SCAgar in 10 mL YDP and grown ON at 30° C. 5 mL ON culture was inoculated in 500 ml FIT media and grown for 5 days at 30° C. Compound of interest was extracted from culture with 500 mL EtAc. Solvent was removed by rotor evaporation and analytes were resuspended in hexane. Extraction was repeated 3 times. Hexane extract was applied on Supelclean™ Florisil®/Na2SO4 SPE Tube (Sigma-Aldrich) and analytes were eluted from column using a step gradient with 1:99-5:95 EtAc: Hexane. Each fraction was analyzed with either LC-MS or GC-MS and the fraction containing the compound of interest was selected for NMR analysis.

### NMR Analysis

NMR data were acquired on a Bruker Avance III HD 600 MHZ NMR spectrometer (1H operating frequency 599.85 MHZ) equipped with a 5 mm cryogenically cooled DCH probe optimized for 13C and 1H (Bruker Biospin, Karlsruhe, Germany). NMR data was recorded in 5 mm tubes in CDCl₃ (Euriso-top, 99.8 atom % D) with temperature equilibration to 300 K, optimization of lock parameters, gradient shimming, and setting of receiver gain, all automatically controlled by Topspin ver. 3.2 and IconNMR ver. 4.7.5 (Bruker Biospin, Karlsruhe, Germany). 1H and 13C chemical shifts were referenced to the residual solvent signals of at respectively pH 7.26 ppm and pC 77.16 ppm. 1D 1H and 13C NMR spectra were acquired with 30° pulses and 64k data points and zero-filled to 256k data points, 1H spectra were acquired with a spectral width of 12 kHz, a relaxation delay of 1 s and an acquisition time of 2.7 s. 13C spectra were 1H-decoupled using the Waltz-16 composite pulse decoupling scheme. 2D homo- and heteronuclear experiments were acquired with 4096 (HMBC), 2048 (DQF-COSY and ROESY), or 1024 (multiplicity edited HSQC) data points in the direct dimension and 256 (DQF-COSY, HMBC and ROESY) or 128 (multiplicity edited HSQC) data points in the indirect dimension. 2D NMR data was zero-filled to 1k in F1 and zero-filled to twice the number of points in F2, employing forward linear prediction in F1 (LPBIN=0). Processing of NMR data was done using Topspin ver. 4.0.9 (Bruker Biospin, Karlsruhe, Germany).

The NMR spectroscopic data for 14-OH-dehydroatietadiene is shown in table 4.

TABLE 4
¹H and ¹³C NMR spectroscopic data of 1 (14-OH-dehydroatietadiene)

| | 14-OH-dehydroabietadiene (1) | | | |
|---|---|---|---|---|
| | | δ_{H}, nH, | | |
| Pos | δ_{C}^{a} | multiplicity (J in Hz)^{a, b} | HMBC | ROESY |
| 1 | 39.1 | A: 2.28, 1H, br d (12.8) | 2, 3, 5, 10, 20 | 2A, 2B, 11, (20) |
| | | B: 1.40, 1H, td (12.8, 3.4) | 2, 3, 9, 10, 20 | (11) |
| 2 | 19.5 | A: 1.75, 1H, m | 1, 3, 4 | 1B |
| | | B: 1.62, 1H, m | 1, 3, 4, 10 | 1B |
| 3 | 41.8 | A: 1.49, 1H, br d (13.2) | 1, 2, 4, 5, 18, 19 | 18 |
| | | B: 1.22, 1H, m | 2, 4, 18, 19 | |
| 4 | 33.5 | — | | |
| 5 | 49.9 | 1.34, 1H, dd, (12.7, 2.1) | 4, 6, 9, 10, 19, 20 | 18 |
| 6 | 18.6 | A: 1.99, 1H, br dd (13.1, 7.9) | 4, 5, 7, 8, 10 | 7B, 18 |
| | | B: 1.72, 1H, m | 5, 7, 10 | 7A, 19 |
| 7 | 24.5 | A: 2.82, 1H, dd (16.5, 6.7) | 5, 6, 8, 9, (14) | 6B, 14-OH |
| | | B: 2.62, 1H, ddd (16.5, 11.4, 7.9) | 6, 8, 9 | 6A, 14-OH |
| 8 | 120.8 | — | | |
| 9 | 149.2 | — | | |
| 10 | 37.7 | — | | |
| 11 | 116.5 | 6.87, 1H, d (8.2) | 8, 10, (12), 13, (14) | 1A, (1B), (20) |
| 12 | 123.4 | 7.02, 1H, d (8.2) | 9, (11), 14, 15 | 16, 17 |
| 13 | 130.1 | — | | |
| 14 | 150.4 | — | | |
| 14-OH | — | 4.63, 1H, s | 8, 13, 14 | 7A, 7B, 15 |
| 15 | 27.0 | 3.16, 1H, sep (6.9) | 12, 13, 14, 16, 17 | 14-OH, 16, 17 |
| 16 | 22.9 | 1.24, 3H, d (6.9) | 13, 15, 17 | 12, 15 |
| 17 | 22.7 | 1.26, 3H, d (6.9) | 13, 15, 16 | 12, 15 |
| 18 | 33.4 | 0.97, 3H, s | 3, 4, 5, 19 | 3B, 5, 6A |
| 19 | 21.8 | 0.94, 3H, s | 3, 4, 5, 18 | 6B, 20 |
| 20 | 25.0 | 1.20, 3H, s | 1, 5, 9, 10 | (1B), (11), 19 |

^{a1}H NMR (599.85 MHz) and ¹³C NMR (150.83 MHz) data obtained in CDCl₃.
^{b}nH = number of hydrogens. Multiplicities reported as apparent splittings: s = singlet, d = doublet, t = triplet, sep = septet, m = multiplet (also in case of overlap), br = broad. ‘A’ denotes the highest chemical shift value and ‘B’ denotes the lowest chemical shift value.

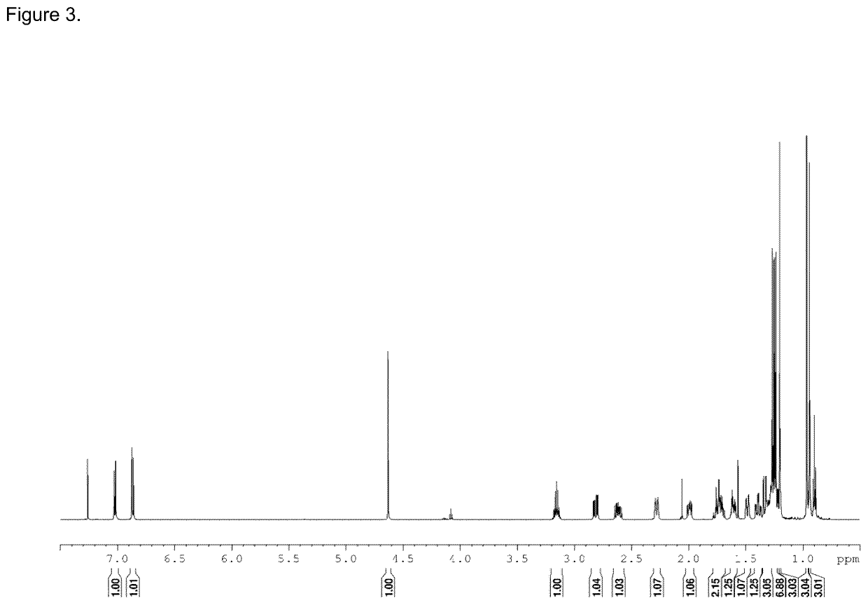

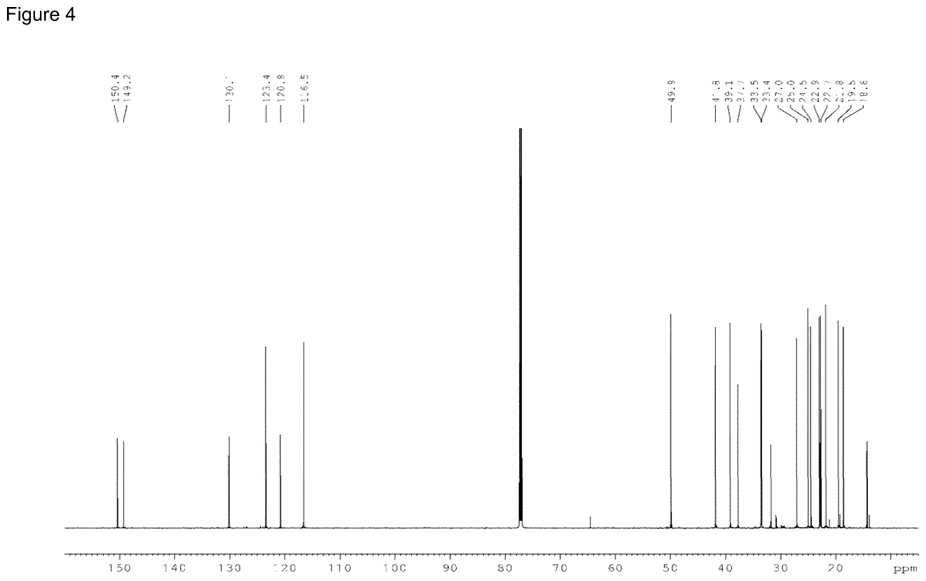

The ¹H NMR spectrum of 14-OH-dehydroatietadiene in CDCl₃ at 599.85 MHz is shown in FIG. 3 and the ¹³C NMR spectrum of 14-OH-dehydroatietadiene in CDCl₃ at 150.83 MHz is shown in FIG. 4 confirming the identity of the compound.

### Example 5: Expression in S. cerevisiae of Genes Leading to Production of Triptophenolide and Triptonide

This was a preliminary study to assess the effects of expression of genes leading to production of triptophenolide and triptonide in S. cerevisiae.

The background yeast strain generated in example 2, was further transformed with vectors each containing individual diterpene biosynthesis genes or Tripterygium wilfordii CYPs (TwCYPs)

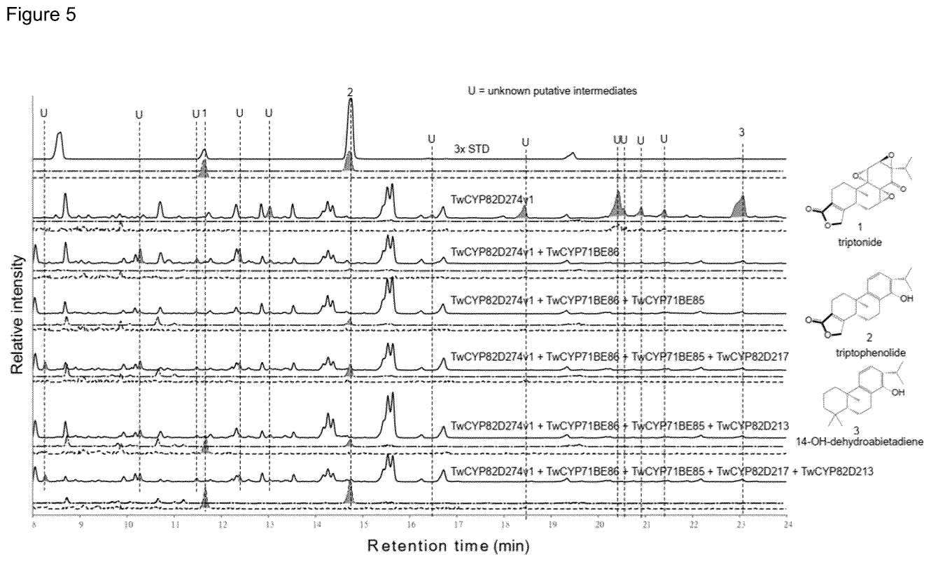

LCMS profiles of the extracts can be seen in FIG. 5, where it can be seen that transforming the background strain with TwCYP82D274V1, TwCYP71BE85 and TwCYP71BE86 encoding the enzymes having the amino acid sequence of SEQ ID NO: 1, SEQ ID NO: 3 and SEQ ID NO: 4, respectively; leads to formation of triptophenolide; and further transformation with CYP82D213 encoding the enzyme having the amino acid sequence of SEQ ID NO: 5, leads to the formation of triptonide.

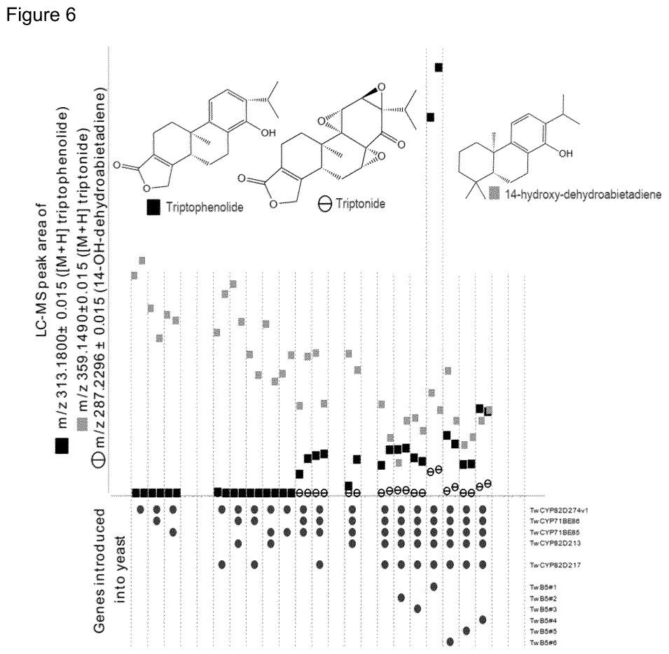

FIG. 6 shows an overview of the content of oxygenated diterpenoid compounds detected in the extracts of the transformants generated.

The left panel shows the content of triptophenolide and triptonide, and the right panel shows the content of 14-OH-dehydroabietadiene. Expressing the gene TwB5 #1 encoding the enzyme having the amino acid sequence of SEQ ID NO: 8 resulted in a significantly higher production of triptophenolide and triptonide.

The genes TwB5 #2-6 are other T. wilfordii cytochrome B5 genes (sequences not provided) that do not increase the production of triptophenolide or triptonide.

### Example 6: Production of Oxygenated Diterpenoid Compounds in S. cerevisiae and N. benthamiana

All engineered S. cerevisiae strains and N. benthamiana were cultured as described herein above in the section “Materials and Methods”. Similarly, diterpenoid metabolites were extracted, analyzed by LC-MS, GC-MS and NMR, and quantified as also described herein above.

It is preferred that the experimental organism is yeast and that the heterologous genes have been stably transfected in the organism as this gives the most precise and reproducible results.

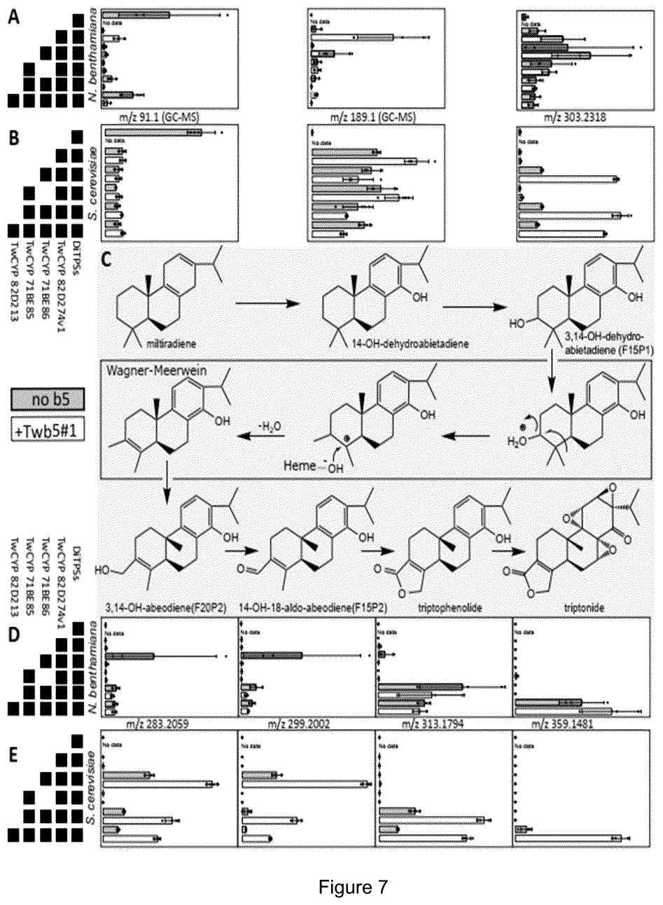

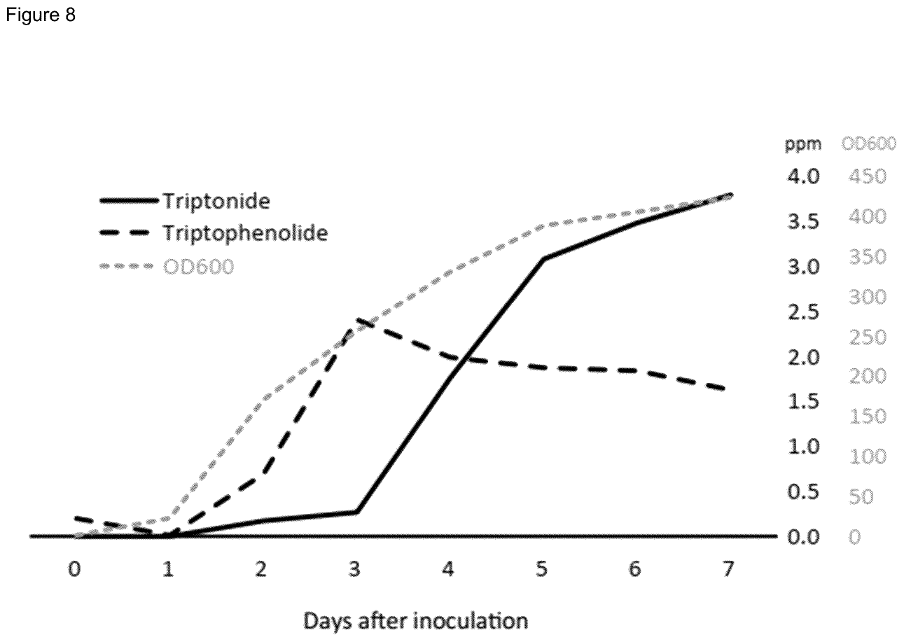

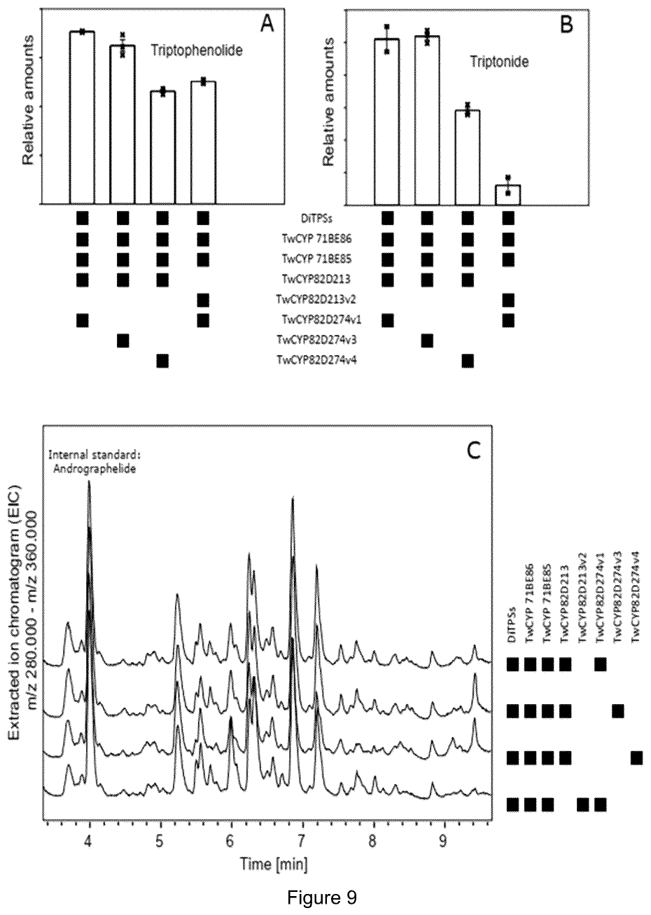

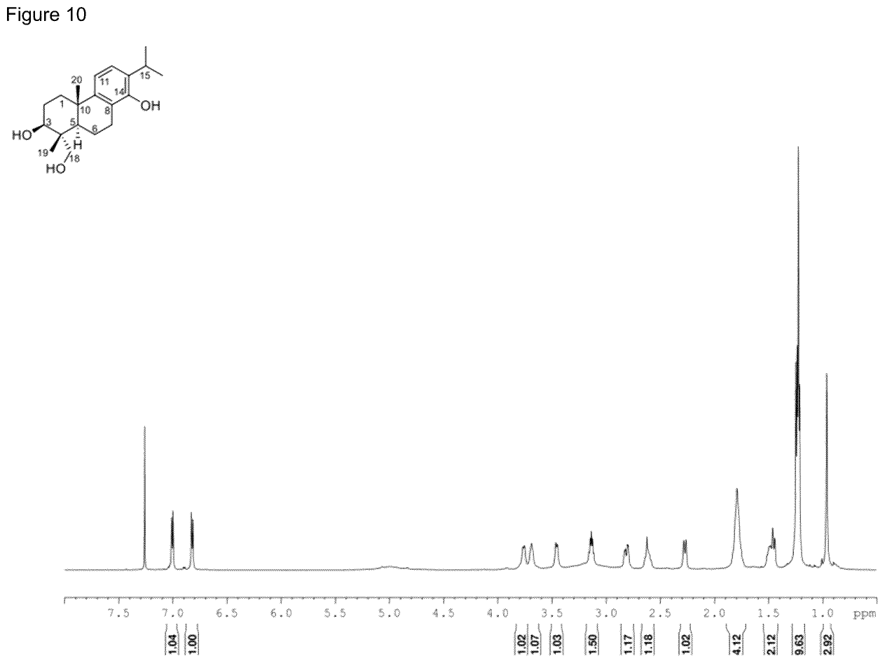

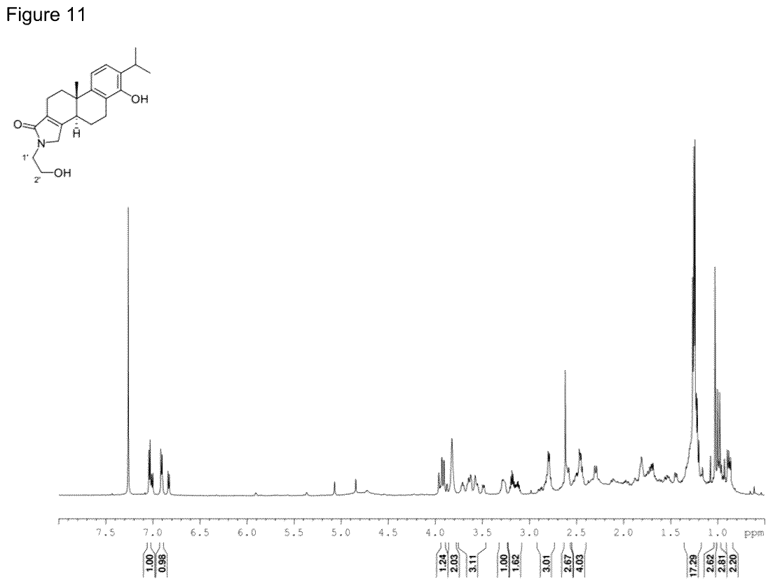

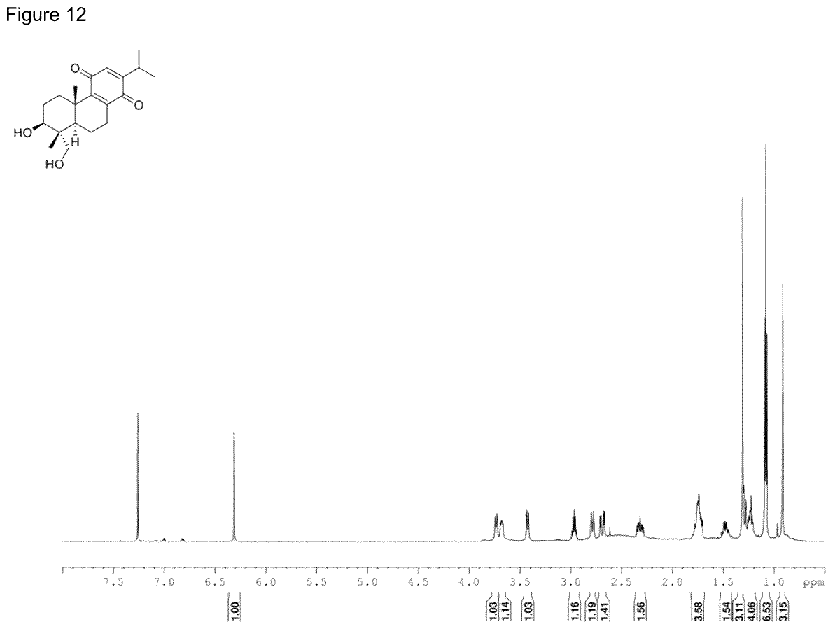

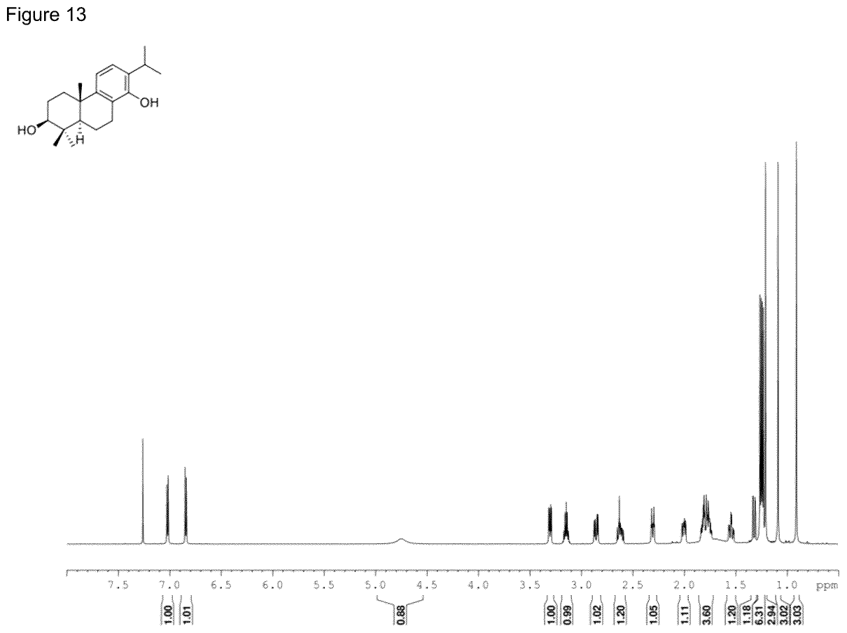

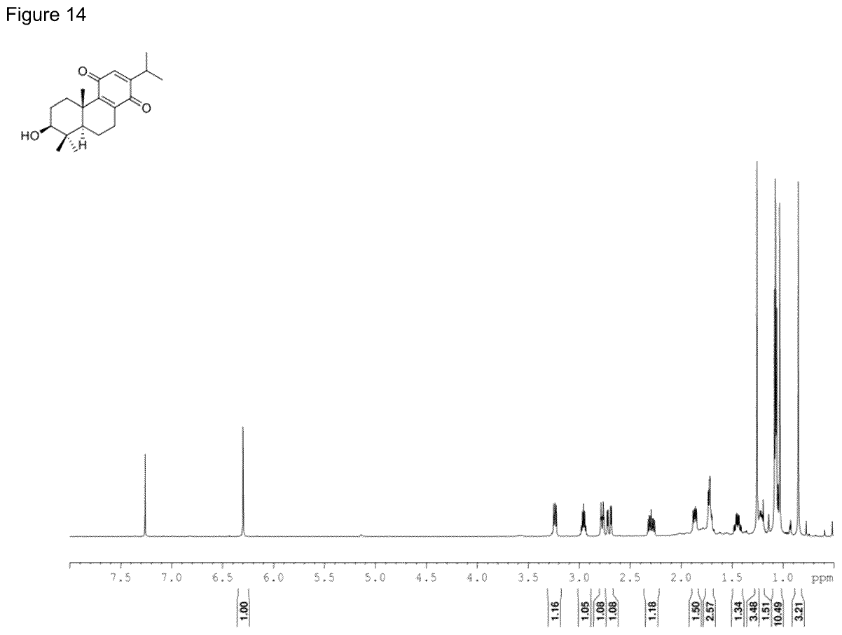

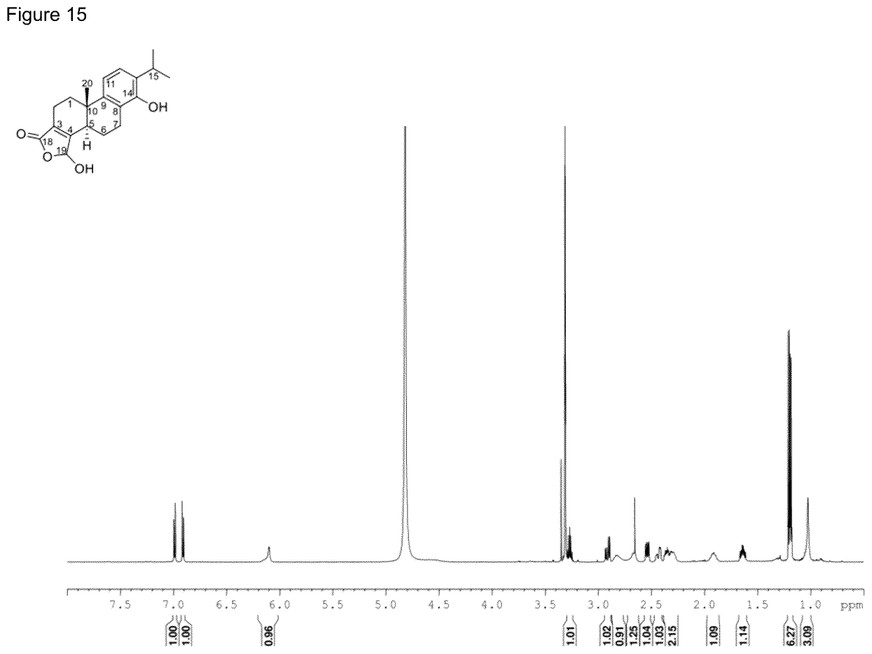

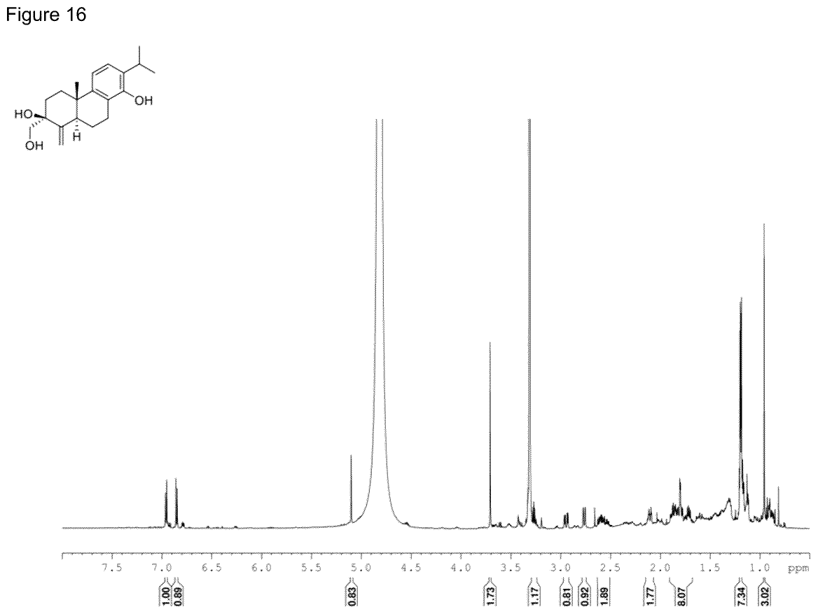

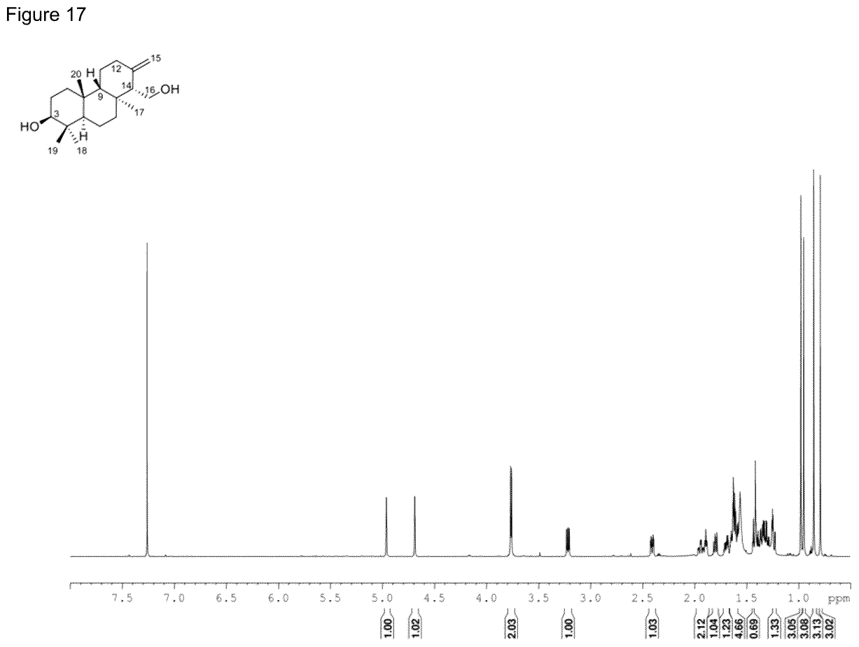

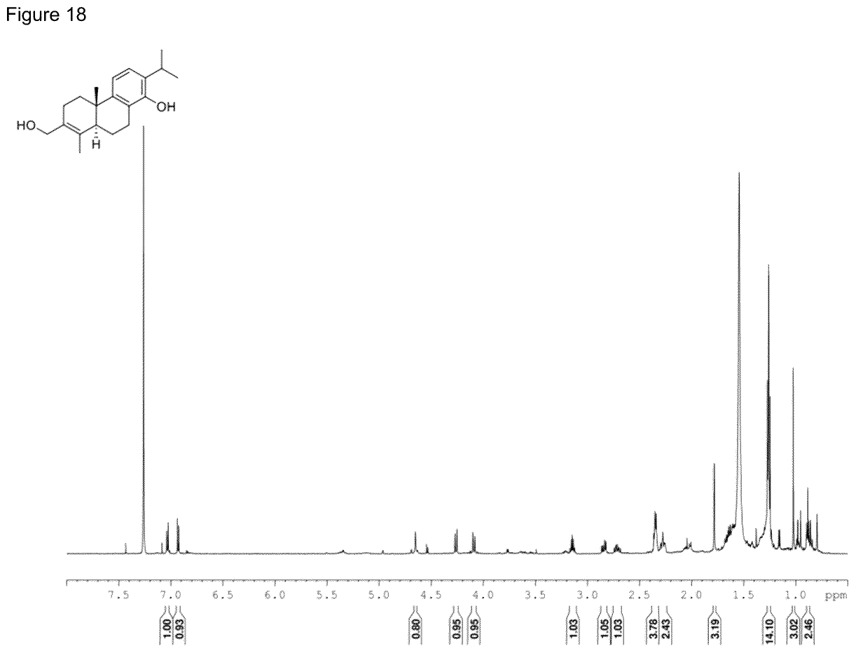

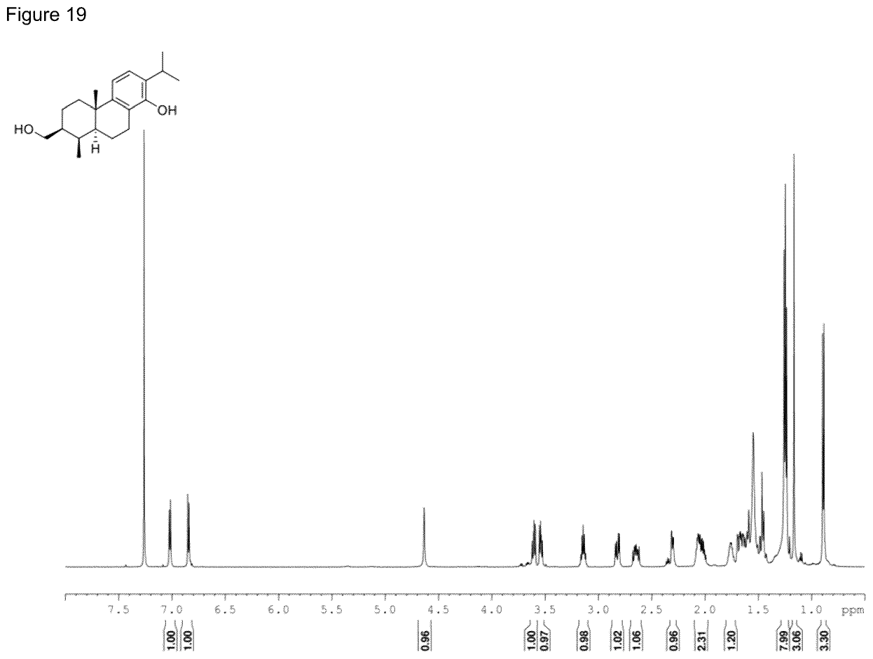

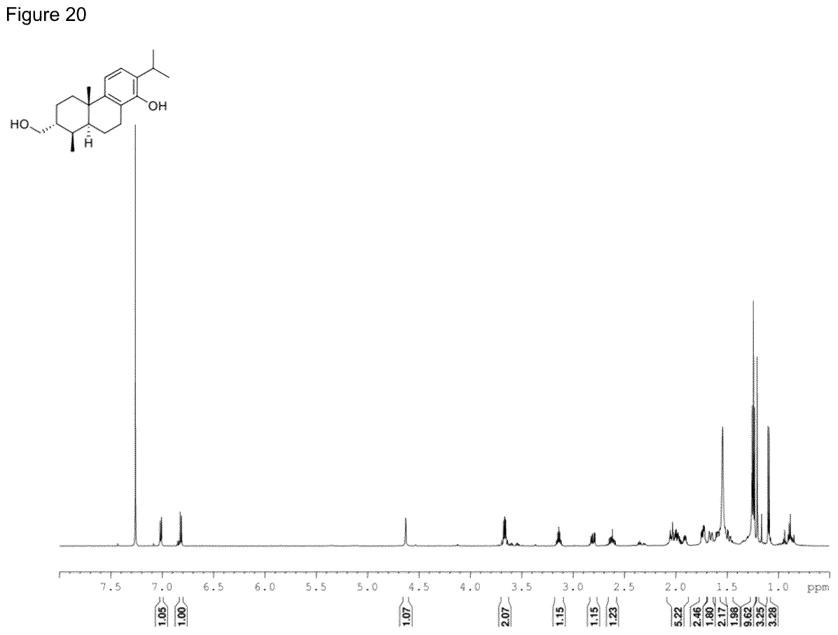

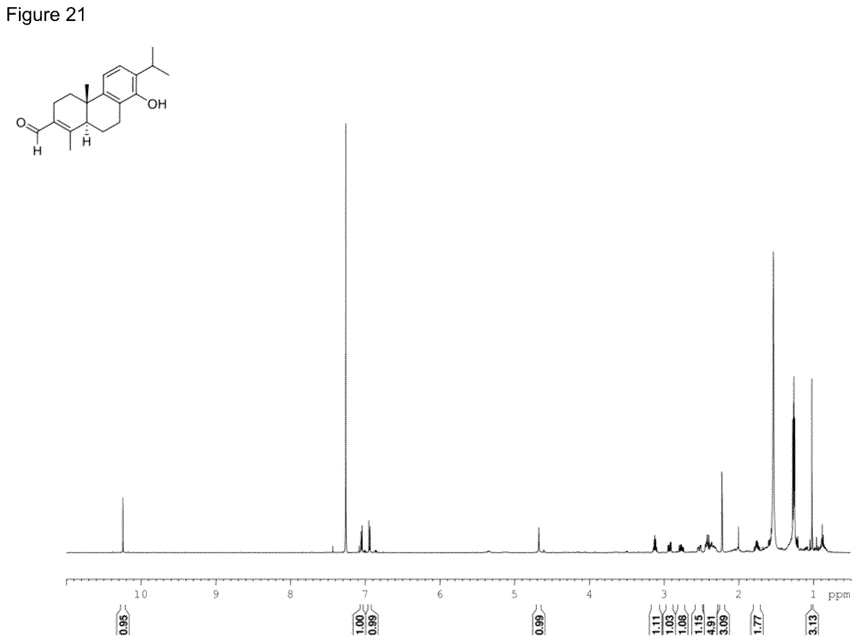

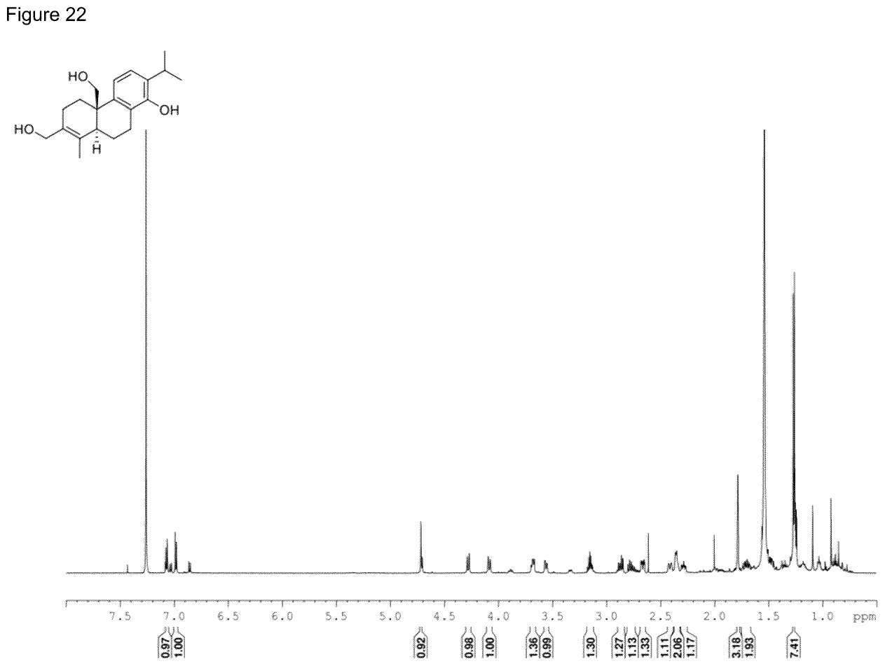

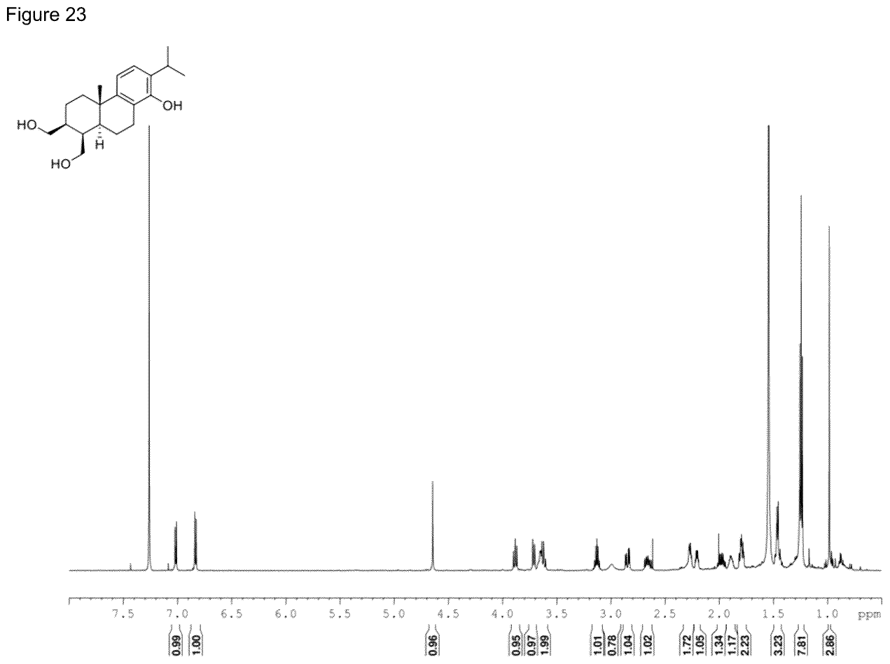

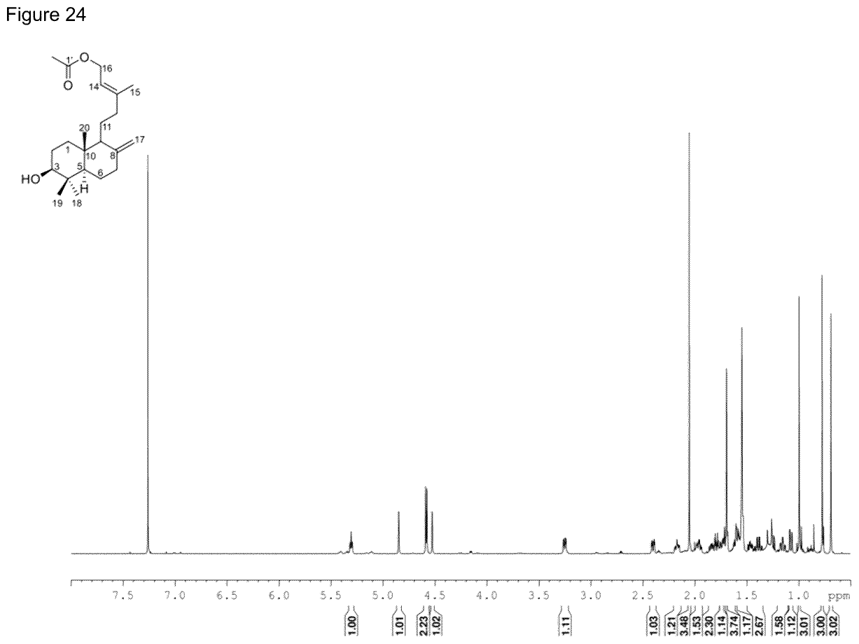

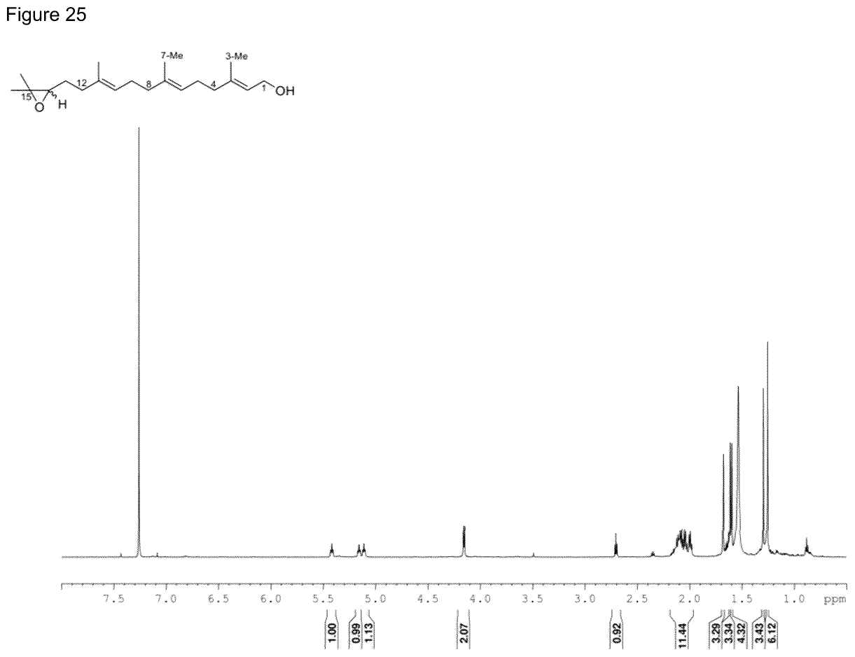

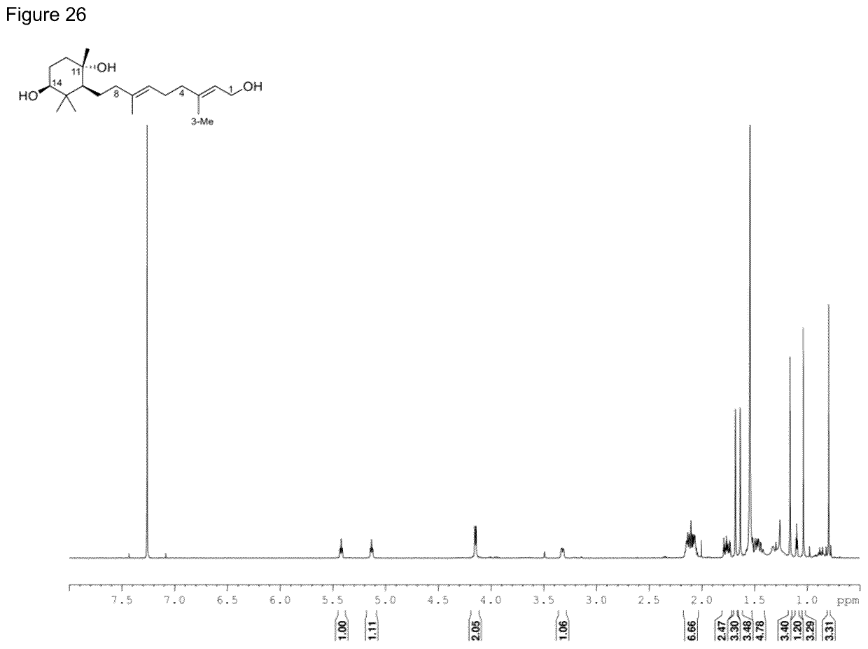

The results are shown in FIGS. 7-9. As can be clearly seen from the figures, organisms as different as yeast cells and tobacco plants are both capable of producing the claimed key intermediates in the proposed biosynthetic pathway of triptonide at high titers according to the methods of the invention.

NMR spectra of other key compounds also produced are shown in FIGS. 10-26. The NMR spectroscopic data for the produced compounds are shown in Tables 5-21, below.

TABLE 5
¹H and ¹³C NMR data and 2D HMBC and ROESY correlations for F1-14

| | δ_{C}, | δ_{H}, nH, | | |
|---|---|---|---|---|
| Pos. | type^{a, b} | multiplicity (J in Hz)^{a, c} | HMBC | ROESY |
| 1 | 36.8, CH₂ | α: 1.49 (1H, m) | C-2, C-3, C-9, C-10, C-20 | H-1β, H-3 |
| | | β: 2.27 (1H, dt, 13.3, 3.1) | C-2, C-3, C-5, C-10, C-20 | H-1α, H-2, H-11, H-20 |
| 2 | 27.3, CH₂ | 1.79 (2H, m) | C-1, C-3, C-4, C-10 | H-1β, H-19, H-20 |
| 3 | 75.9, CH | 3.69 (1H, t, 7.5) | C-2, C-4, C-18, C-19 | H-1α, H-5 |
| 4 | 42.0, C | — | | |
| 5 | 43.5, CH | 1.45 (1H, br d, 11.8) | C-1, C-4, C-6, C-7, C-9, | H-3, H-7A, H-18A |
| | | | C-10, C-18, C-20 | |
| 6 | 18.2, CH₂ | A: 1.75 (1H, m) | C-5, C-7, C-10 | |
| | | B: 1.80 (1H, m) | C-8, C-10 | |
| 7 | 24.3, CH₂ | A: 2.62 (1H, m) | C-6, C-8, C-9, C-14 | H-5 |
| | | B: 2.82 (1H, dd, 16.2, 5.1) | C-5, C-6, C-8, C-9, C-14 | |
| 8 | 120.7, C | — | | |
| 9 | 147.9, C | — | | |
| 10 | 37.1, C | — | | |
| 11 | 116.4, CH | 6.82 (1H, d, 8.2) | C-8, C-10, C-13 | H-1β, H-20 |
| 12 | 123.3, CH | 7.01 (1H, d, 8.2) | C-9, C-14, C-15 | H-15, H-16, H-17 |
| 13 | 130.4, C | — | | |
| 14 | 150.2, C | — | | |
| 15 | 26.7, CH | 3.14 (1H, sep, 6.9) | C-12, C-13, C-14, C-16, C-17 | H-12 |
| 16 | 22.7, CH₃ | 1.22 (3H, d, 6.9) | C-13, C-15, C-17 | H-12 |
| 17 | 22.6, CH₃ | 1.24 (3H, d, 6.9) | C-13, C-15, C-16 | H-12 |
| 18 | 71.0, CH₂ | A: 3.46 (1H, d, 8.9) | C-3, C-4, C-5, C-19 | H-5 |
| | | B: 3.76 (1H, d, 8.9) | C-3, C-4, C-5 | |
| 19 | 11.2, CH₃ | 0.96 (3H, s) | C-3, C-4, C-5, C-18 | H-2, H-20 |
| 20 | 25.2, CH₃ | 1.22 (3H, s) | C-1, C-5, C-9, C-10 | H-1β, H-2, H-11, H-19 |

TABLE 6
¹H and ¹³C NMR data and 2D HMBC and ROESY correlations for F1-15

| | δ_{C}, | δ_{H}, nH, | | |
|---|---|---|---|---|
| Pos. | type^{a, b} | multiplicity (J in Hz)^{a, c} | HMBC | ROESY |
| 1 | 32.7, CH₂ | α: 1.69 (1H, m) | C-2, C-10, C-20 | H-1β, H-2B, H-5 |
| | | β: 2.46 (1H, m) | C-2, C-5, C-9, C-20 | H-1α, H-11, H-20 |
| 2 | 22.5, CH₂ | A: 2.45 (1H, m) | C-1, C-3, C-4, C-10 | H-19B |
| | | B: 2.52 (1H, m) | C-1, C-3, C-4 | H-1α, H-19A |
| 3 | 132.0, C | — | | |
| 4 | 150.7, C | — | | |
| 5 | 39.2, CH | 2.60 (1H, m) | | H-1α, H-6α, H-7B |
| 6 | 17.5, CH₂ | α: 3.28 (1H, m) | C-8, C-10 | H-5, H-7B |
| | | β: 1.73 (1H, m) | C-5, C-7, C-10 | H-20 |
| 7 | 22.5, CH₂ | A: 2.46 (1H, m) | | |
| | | B: 2.80 (1H, td, 9.5, 7.6) | C-5, C-6, C-8, C-9, C-14 | H-6α, H5 |
| 8 | 120.8, C | — | | |
| 9 | 144.5, C | — | | |
| 10 | 36.1, C | — | | |
| 11 | 115.7, CH | 6.91 (1H, d, 8.1) | C-8, C-10, C-12, C-13 | H-1β, H-20 |
| 12 | 123.0, CH | 7.04 (1H, d, 8.1) | C-9, C-11, C-14, C-15 | H-16, H-17 |
| 13 | 131.2, C | — | | |
| 14 | 150.9, C | — | | |
| 15 | 26.9, CH | 3.18 (1H, sep, 7.0) | C-12, C-13, C-14, C-16, C-17 | |
| 16 | 22.6, CH₃ | 1.24 (3H, d, 7.0) | C-13, C-15, C-17 | H-12 |
| 17 | 22.6, CH₃ | 1.26 (3H, d, 7.0) | C-13, C-15, C-16 | H-12 |
| 18 | 173.5, C | — | | |
| 19 | 53.9, CH₂ | A: 3.90 (1H, dd, 18.7, 2.1) | C-3, C-4, C-18 | H-2B, H-1′ |
| | | B: 3.94 (1H, dd, 18.7, 2.7) | C-3, C-4, C-18 | H-2A, H-1′ |
| 20 | 22.4, CH₃ | 1.03 (3H, s) | C-1, C-5, C-9, C-10 | H-1β, H-6β, H-11 |
| 1′ | 46.1, CH₂ | A: 3.56 (1H, m) | C-18, C-19, C-2′ | H-19 |
| | | B: 3.63 (1H, m) | C-18, C-19, C-2′ | H-19 |
| 2′ | 62.2, CH₂ | 3.82 (2H, m) | C-1′ | |

TABLE 7
¹H and ¹³C NMR data and 2D HMBC and ROESY correlations for F1-18

| Pos. | δ_{C}, type^{a, b} | δ_{H}, nH, multiplicity (J in Hz)^{a, c} | HMBC | ROESY |
|---|---|---|---|---|
| 1 | 34.0, CH₂ | α: 1.23 (1H, m) | C-2, C-10, C-20 | H-1β, H-3 |
| | | β: 2.79 (1H, dt, 13.5, 3.3) | C-3, C-5, C-10 | H-1α, H-2, H-20 |
| 2 | 27.1, CH₂ | 1.76 (2H, m) | C-1, C-3, C-4, C-10 | H-1β, H-20 |
| 3 | 75.1, CH | 3.68 (1H, dd, 10.3, 5.8) | C-2, C-4, C-18, C-19 | H-1α, H-5, H-18A |
| 4 | 42.0, C | — | | |
| 5 | 45.2, CH | 1.29 (1H, d, 12.5) | C-4, C-6, C-7, C-10, C-18, C-19, C-20 | H-3, H-7B, H-18A |
| 6 | 17.1, CH₂ | α: 1.73 (1H, m) | C-5, C-7, C-8, C-10 | H-7B, H-18A, H-18B |
| | | β: 1.48 (1H, qd, 12.5, 5.7) | C-5, C-7, C-10 | H-7B, H-19 |
| 7 | 25.8, CH₂ | A: 2.32 (1H, ddd, 20.2, 11.6, 7.4) | C-6, C-8, C-9, C-14 | H-7B, H-6α |
| | | B: 2.69 (1H, dd, 20.2, 5.5) | C-5, C-6, C-8, C-9, C-14 | H-5, H-6α, H-6β, H-7A |
| 8 | 142.6, C | — | | |
| 9 | 149.9, C | — | | |
| 10 | 38.0, C | — | | |
| 11 | 187.7, C | — | | |
| 12 | 131.8, CH | 6.31 (1H, s) | C-9, C-11, C-14, C-15 | H-15, H-16/17 |
| 13 | 152.9, C | — | | |
| 14 | 187.6, C | — | | |
| 15 | 26.2, CH | 2.97 (1H, sep, 6.9) | C-12, C-13, C-14, C-16, C-17 | H-12 |
| 16 | 21.2, CH₃ | 1.08 (3H, d, 6.9) | C-13, C-15, C-17 | H-12 |
| 17 | 21.2, CH₃ | 1.09 (3H, d, 6.9) | C-13, C-15, C-16 | H-12 |
| 18 | 70.4, CH₂ | A: 3.42 (1H, d, 10.3) | C-3, C-4, C-5, C-19 | H-3, H-5, H-6α, H-19 |
| | | B: 3.74 (1H, d, 10.3) | C-3, C-4, C-5, C-19 | H-6α, H-19 |
| 19 | 11.6, CH₃ | 0.92 (3H, s) | C-3, C-4, C-5, C-18 | H-6β, H-18A, H-18B, H-20 |
| 20 | 20.4, CH₃ | 1.31 (3H, s) | C-1, C-5, C-9, C-10 | H-1β, H-2, H-19 |

TABLE 8
¹H and ¹³C NMR data and 2D HMBC and ROESY correlations for F1-23 (F15P1)

| Pos. | δ_{C}, type^{a, b} | δ_{H}, nH, multiplicity (J in Hz)^{a, c} | HMBC | ROESY |
|---|---|---|---|---|
| 1 | 37.1, CH₂ | α: 1.55 (1H, td, 13.2, 4.2) | C-2, C-3, C-9, C-10, C-20 | H-3 |
| | | β: 2.31 (1H, dt, 13.2, 3.5) | C-2, C-3, C-5, C-10, C-20 | H-2, H-11, H-20 |
| 2 | 27.9, CH₂ | 1.80 (2H, m) | C-1, C-3, C-4, C-10 | H-1β |
| 3 | 78.7, CH | 3.31 (1H, dd, 11.5, 4.7) | C-2, C-4, C-18, C-19 | H-1α, H-5, H-18 |
| 4 | 38.9, C | | | |
| 5 | 49.2, CH | 1.32 (1H, dd, 12.5, 2.0) | C-3, C-4, C-6, C-7, C-9, C-10, | H-1α, H-3, H-7α, H-18 |
| | | | C-18, C-19, C-20 | |
| 6 | 18.2, CH₂ | α: 2.00 (1H, ddt, 13.3, 7.9, 2.0) | C-4, C-5, C-7, C-8, C-10 | H-7α, H-7β, H-18, H-19 |
| | | β: 1.77 (1H, m) | C-5, C-7, C-10 | H-7β, H-19 |
| 7 | 24.6, CH₂ | α: 2.62 (1H, ddd, 16.7, 11.6, 7.9) | C-6, C-8, C-9, C-14 | H-5, H-6α |
| | | β: 2.86 (1H, dd, 16.7, 6.5) | C-5, C-6, C-8, C-9, C-14 | H-6α, H-6β |
| 8 | 120.6, C | — | | |
| 9 | 148.2, C | — | | |
| 10 | 37.3, C | — | | |
| 11 | 116.4, CH | 6.84 (1H, d, 8.3) | C-8, C-10, C-13 | H-1β |
| 12 | 123.3, CH | 7.02 (1H, d, 8.3) | C-9, C-14, C-15 | H-16, H-17 |
| 13 | 130.2, C | — | | |
| 14 | 150.2, C | — | | |
| 15 | 26.7, CH | 3.15 (1H, sep, 6.9) | C-12, C-13, C-14, C-16, C-17 | |
| 16 | 22.7, CH₃ | 1.24 (3H, d, 6.9) | C-13, C-15, C-17 | H-12 |
| 17 | 22.5, CH₃ | 1.26 (3H, d, 6.9) | C-13, C-15, C-16 | H-12 |
| 18 | 28.1, CH₃ | 1.09 (3H, s) | C-3, C-4, C-5, C-19 | H-3, H-5, H-6α |
| 19 | 15.3, CH₃ | 0.91 (3H, s) | C-3, C-4, C-5, C-18 | H-6α, H-6β, H-20 |
| 20 | 24.8, CH₃ | 1.21 (3H, s) | C-1, C-5, C-9, C-10 | H-1β, H-19 |

TABLE 9
¹H and ¹³C NMR data and 2D HMBC and ROESY correlations for F1-31

| Pos. | δ_{C}, type^{a, b} | δ_{H}, nH, multiplicity (J in Hz)^{a, c} | HMBC | ROESY |
|---|---|---|---|---|
| 1 | 34.4, CH₂ | α: 1.22 (1H, m) | C-2, C-3, C-9, C-10, C-20 | H-3 |
| | | β: 2.78 (1H, dt, 13.5, 3.6) | C-3, C-5, C-10 | H-2, H-20 |
| 2 | 27.7, CH₂ | 1.72 (2H, m) | C-1, C-3, C-4, C-10 | H-1β, H-19 |
| 3 | 78.3, CH | 3.24 (1H, m) | C-2, C-4, C-18, C-19 | H-1α, H-18 |
| 4 | 39.0, C | — | | |
| 5 | 51.0, CH | 1.06 (1H, m) | C-4, C-6, C-10, C-19, C-20 | H-3, H-6α, H-7B, H-7A |
| 6 | 17.1, CH₂ | α: 1.87 (1H, br dd, 13.5, 7.5) | C-4, C-5, C-7, C-8, C-10 | H-5, H-7B, H-18 |
| | | β: 1.45 (1H, dtd, 13.5, 11.7, 5.7) | C-5, C-7, C-10 | H-7B, H-19 |
| 7 | 26.1, CH₂ | A: 2.30 (1H, ddd, 20.2, 11.7, 7.5) | C-6, C-8, C-9, C-14 | H-5, H-6α |
| | | B: 2.71 (1H, br dd, 20.2, 5.7) | C-5, C-6, C-8, C-9, C-14 | H-5, H-6α, H-6β |
| 8 | 142.7, C | — | | |
| 9 | 150.0, C | — | | |
| 10 | 38.1, C | — | | |
| 11 | 187.78, C | — | | |
| 12 | 131.8, CH | 6.30 (1H, d, 1.0) | C-9, C-11/C-14, C-15 | H-16/17 |
| 13 | 152.9, C | — | | |
| 14 | 187.83, C | — | | |
| 15 | 26.2, CH | 2.96 (1H, sep d, 6.9, 1.0) | C-12, C-13, C-14, C-16, C-17 | |
| 16 | 21.3, CH₃ | 1.07 (3H, d, 6.9) | C-13, C-15, C-17 | H-12 |
| 17 | 21.3, CH₃ | 1.08 (3H, d, 6.9) | C-13, C-15, C-16 | H-12 |
| 18 | 28.2, CH₃ | 1.03 (3H, s) | C-3, C-4, C-5, C-19 | H-3, H-6α |
| 19 | 15.7, CH₃ | 0.85 (3H, s) | C-3, C-4, C-5, C-18 | H-2, H-6β, H-20 |
| 20 | 20.1, CH₃ | 1.26 (3H, s) | C-1, C-5, C-9, C-10 | H-1β, H-19 |

TABLE 10
¹H and ¹³C NMR data and 2D HMBC and ROESY correlations for F2-X

| Pos. | δ_{C}, type^{a, b} | δ_{H}, nH, multiplicity (J in Hz)^{a, c} | HMBC | ROESY |
|---|---|---|---|---|
| 1 | 33.5, CH₂ | α: 1.64 (1H, ddd, 13.2, 11.5, 6.5) | C-2, C-5, C-9, C-10, C-20 | H-1β, H-2B, H-5 |
| | | β: 2.54 (1H, dd, 13.2, 6.2) | C-2, C-3, C-5, C-9, C-10, (C-18), C-20 | H-1α, H-11, H-20 |
| 2 | 18.6, CH₂ | A: 2.34 (1H, m) | C-1, C-3, C-4 | H-20 |
| | | B: 2.43 (1H, dd, 18.1, 5.4) | C-1, C-3, C-4, C-10 | H-1α, H-20 |
| 3 | 128.6, C | — | | |
| 4 | 164.0, C | — | | |
| 5 | 41.4, CH | 2.67 (1H, br s) | | H-1α, H-6α |
| 6 | 19.8, CH₂ | α: 2.30 (1H, m) | | H-5, H-6β, H-7A, H-7B, H-19 |
| | | β: 1.91 (1H, m) | | H-6α, H-7B, H-19, H-20 |
| 7 | 23.8, CH₂ | A: 2.83 (1H, m) | | H-6α |
| | | B: 2.91 (1H, dd, 17.8, 7.5) | C-5, C-6, C-8, C-9, C-14 | H-6α, H-6β |
| 8 | 123.2, C | — | | |
| 9 | 144.7, C | — | | |
| 10 | 37.0, C | — | | |
| 11 | 116.8, CH | 6.91 (1H, d, 8.2) | C-8, C-10, C-13 | H-1β |
| 12 | 123.7, CH | 6.99 (1H, d, 8.2) | C-9, C-14, C-15 | H-16, H-17 |
| 13 | 133.7, C | — | | |
| 14 | 152.4, C | — | | |
| 15 | 27.4, CH | 3.27 (1H, sep, 6.9) | C-12, C-13, C-14, C-16, C-17 | |
| 16 | 23.1, CH₃ | 1.18 (3H, d, 6.9) | C-13, C-15, C-17 | H-12 |
| 17 | 23.0, CH₃ | 1.20 (3H, d, 6.9) | C-13, C-15, C-16 | H-12 |
| 18 | 173.4, C | — | | |
| 19 | 99.4, CH | 6.10 (1H, br s) | | H-6α, H-6β |
| 20 | 22.6, CH₃ | 1.02 (3H, br s) | C-1, C-5, C-9, C-10 | H-1β, H-2A, H-2B, H-6β |

TABLE 11
¹H and ¹³C NMR data and 2D HMBC and ROESY correlations for F2-10

| Pos. | δ_{C}, type^{a, b} | δ_{H}, nH, multiplicity (J in Hz)^{a, c} | HMBC | ROESY |
|---|---|---|---|---|
| 1 | 34.8, CH₂ | α: 1.87 (1H, td, 12.7, 5.2) | C-2, C-10, C-20 | H-5, H-18 |
| | | β: 2.10 (1H, dt, 12.7, 3.6) | C-2, C-3, C-5 | H-2, H-11, H-20 |
| 2 | 32.4, CH₂ | 1.80 (2H, m) | C-1, C-3, C-4, C-10 | H-1β, H-5, H-18, H-20 |
| 3 | 73.4, C | — | | |
| 4 | 152.5, C | — | | |
| 5 | 43.4, CH | 2.76 (1H, br d, 12.3) | C-4, C-6, C-10, C-19 | H-1α, H-2, H-7α |
| 6 | 21.7, CH₂ | α: 1.84 (1H, m) | C-5, C-7, C-8, C-10 | H-7α, H-7β, H-19A |
| | | β: 1.71 (1H, m) | C-5, C-7, C-10 | H-7β, H-19A, H-20 |
| 7 | 25.2, CH₂ | α: 2.60 (1H, m) | C-6, C-8, C-9, C-14 | H-5, H-6α, H-6β, H-7B |
| | | β: 2.94 (1H, dd, 17.2, 5.8) | C-5, C-6, C-8, C-9, C-14 | H-6α, H-6β, H-7α |
| 8 | 123.5, C | — | | |
| 9 | 146.4, C | — | | |
| 10 | 39.8, C | — | | |
| 11 | 118.2, CH | 6.85 (1H, d, 8.2) | C-8, C-10, C-13 | H-1β |
| 12 | 123.6, CH | 6.96 (1H, d, 8.2) | C-9, C-14, C-15 | H-16/17 |
| 13 | 132.9, C | | | |
| 14 | 151.8, C | | | |
| 15 | 27.3, CH | 3.27 (1H, sep, 6.9) | C-12, C-13, C-14, C-16/17 | |
| 16 | 23.0, CH₃ | 1.19 (3H, d, 6.9) | C-13, C-15, C-17 | H-12 |
| 17 | 23.0, CH₃ | 1.19 (3H, d, 6.9) | C-13, C-15, C-16 | H-12 |
| 18 | 68.6, CH₂ | 3.71 (2H, s) | C-2, C-3, C-4 | H-1α, H-2, H-19B |
| 19 | 107.5, CH₂ | A: 4.83 (1H, br s) | C-3, C-4, C-5 | H-6α, H-6β |
| | | B: 5.10 (1H, br s) | C-3, C-4, C-5 | H-18 |
| 20 | 21.8, CH₃ | 0.96 (3H, s) | C-1, C-5, C-9, C-10 | H-1β, H-2, H-6β |

TABLE 12
¹H and ¹³C NMR data and 2D HMBC and ROESY correlations for F20P1

| Pos. | δ_{C}, type^{a, b} | δ_{H}, nH, multiplicity (J in Hz)^{a, c} | HMBC | ROESY |
|---|---|---|---|---|
| 1 | 33.1, CH₂ | A: 1.35 (1H, m) | C-2, C-3, C-5, C-10, C-20 | H-2B |
| | | B: 1.427 (1H, m) | | H-3, H-17, H-18 |
| 2 | 28.9, CH₂ | A: 1.60 (1H, m) | C-1, C-3 | |
| | | B: 1.69 (1H, m) | | H-1A |
| 3 | 79.0, CH | 3.22 (1H, dd, 11.8, 5.0) | C-2, C-4, C-18, C-19 | H-1B, H-5, H-18 |
| 4 | 38.9, C | — | | |
| 5 | 46.0, CH | 1.415 (1H, m) | C-3, C-4, C-6, C-7, C-10, C-18, C-19, C-20 | H-3, H-18 |
| 6 | 17.5, CH₂ | A: 1.407 (1H, m) | C-5 | H-19 |
| | | B: 1.62 (1H, m) | C-4, C-5, C-7, C-8, C-10 | H-19 |
| 7 | 38.6, CH₂ | A: 1.62 (1H, m) | C-6, C-9 | |
| | | B: 1.80 (1H, dd, 11.5, 7.6) | C-5, C-6, C-8, C-14, C-17 | |
| 8 | 37.9, C | — | | |
| 9 | 53.6, CH | 1.24 (1H, dd, 12.9, 3.0) | C-1, C-8, C-10, C-11, C-12, C-17, C-20 | H-14, H-20 |
| 10 | 36.9, C | — | | |
| 11 | 24.5, CH₂ | A: 1.32 (1H, qd, 12.9, 4.0) | C-9, C-12 | H-12A, H-12B, H-17 |
| | | B: 1.64 (1H, m) | C-9 | H-12A, H-12B, H-20 |
| 12 | 37.8, CH₂ | A: 1.94 (1H, tdt, 12.8, 4.6, ~1) | C-11, C-13, C-15 | H-11A, H-11B |
| | | B: 2.41 (1H, ddd, 12.8, 4.0, 2.6) | C-9, C-11, C-13, C-14, C-15 | H-11A, H-11B, H-15B |
| 13 | 147.5, C | — | | |
| 14 | 60.8, CH | 1.89 (1H, t, 6.5) | C-8, C-9, C-13, C-15, C-16, C-17 | H-9 |
| 15 | 107.0, CH₂ | A: 4.69 (1H, dt, 1.4, 1.2) | C-12, C-13, C-14 | H-15B, H-16 |
| | | B: 4.96 (1H, dt, 1.4, 1.2) | C-12, C-13, C-14 | H-12B, H-15A |
| 16 | 59.0, CH₂ | 3.76 (2H, d, 6.5) | C-8, C-13, C-14 | H-7A, H-7B, H-15A, H-17 |
| 17 | 21.0, CH₃ | 0.85 (3H, s) | C-7, C-8, C-9, C-14 | H-1B, H-11A, H-16 |
| 18 | 29.0, CH₃ | 0.98 (3H, s) | C-3, C-4, C-5, C-19 | H-1B, H-3, H-5, H-6B |
| 19 | 15.8, CH₃ | 0.79 (3H, s) | C-3, C-4, C-5, C-18 | H-6A, H-6B |
| 20 | 22.3, CH₃ | 0.95 (3H, s) | C-1, C-5, C-9, C-10 | H-9, H-11B |

TABLE 13
¹H and ¹³C NMR data and 2D HMBC and ROESY correlations for F20P2

| Pos. | δ_{C}, type^{a, b} | δ_{H}, nH, multiplicity (J in Hz)^{a, c} | HMBC | ROESY |
|---|---|---|---|---|
| 1 | 33.4, CH₂ | α: 1.60 (1H, m) | | H-1β, H-5 |
| | | β: 2.35 (1H, m) | C-5, C-10 | H-1α, H-11, H-20 |
| 2 | 26.0, CH₂ | 2.35 (2H, m) | C-3, C-4 | H-18A, H-18B |
| 3 | 131.8, C | — | | |
| 4 | 129.7, C | — | | |
| 5 | 44.5, CH | 2.27 (1H, m) | | H-1α |
| 6 | 20.0, CH₂ | α: 2.28 (1H, m) | C-8, C-10 | H-7A, H-7B, H-19 |
| | | β: 1.64 (1H, m) | (C-5) | H-7B, H-20 |
| 7 | 23.3, CH₂ | A: 2.71 (1H, ddd, 16.8, 10.9, 8.4) | C-6, C-8 | H-6α, 14-OH |
| | | B: 2.84 (1H, dd, 16.8, 7.1) | C-5, C-6, C-8, C-9, C-14 | H-6α, H-6β, 14-OH |
| 8 | 120.6, C | — | | |
| 9 | 146.0, C | — | | |
| 10 | 35.6, C | — | | |
| 11 | 116.2, CH | 6.93 (1H, d, 8.1) | C-8, C-10, C-13 | H-1β |
| 12 | 123.1, CH | 7.03 (1H, d, 8.1) | C-9, C-14, C-15 | H-16, H-17 |
| 13 | 130.3, C | — | | |
| 14 | 150.4, C | — | | |
| 14-OH | — | 4.65 (1H, br s) | | H-7α, H-7β, H-15 |
| 15 | 26.9, CH | 3.14 (1H, sep, 6.9) | C-12, C-13, C-14, C-16, C-17 | 14-OH |
| 16 | 22.6, CH₃ | 1.25 (3H, d, 6.9) | C-13, C-15, C-17 | H-12 |
| 17 | 22.6, CH₃ | 1.27 (3H, d, 6.9) | C-13, C-15, C-16 | H-12 |
| 18 | 63.4, CH₂ | A: 4.09 (1H, d, 11.6) | C-2, C-3, C-4 | H-2, H-19 |
| | | B: 4.26 (1H, d, 11.6) | C-2, C-3, C-4 | H-2, H-19 |
| 19 | 15.6, CH₃ | 1.78 (3H, br s) | C-3, C-4, C-5 | H-6α, H-18A, H-18B |
| 20 | 22.4, CH₃ | 1.02 (3H, s) | C-1, C-5, C-9, C-10 | H-1β, H-6β |

TABLE 14
¹H and ¹³C NMR data and 2D HMBC and ROESY correlations for F20P3

| Pos. | δ_{C}, type^{a, b} | δ_{H}, nH, multiplicity (J in Hz)^{a, c} | HMBC | ROESY |
|---|---|---|---|---|
| 1 | 38.5, CH₂ | α: 1.46 (1H, m) | C-2, C-10, C-20 | H1β, H-3 |
| | | β: 2.30 (1H, m) | C-2, C-3, C-5, C-10 | H1α, H-2β, H-11, H-20 |
| 2 | 20.7, CH₂ | α: 1.60 (1H, m) | | H-18A, H-20 |
| | | β: 1.47 (1H, m) | C-1, C-3 | H1β, H-18B, H-19 |
| 3 | 44.4, CH | 1.76 (1H, m) | | H1α, H-4, H-18A, H-18B |
| 4 | 35.0, CH | 2.07 (1H, m) | C-2, C-3, C-5, C-10, C-19 | H-3, H-5, H-6α, H-18B |
| 5 | 44.7, CH | 1.68 (1H, ddd, 12.8, 4.4, 2.1) | C-4, C-6, C-7, C-10, C-19, C-20 | H-4, H-7α |
| 6 | 23.7, CH₂ | α: 1.65 (1H, br dd, 12.8, 8.2) | C-4, C-5, C-7, C-8, C-10 | H-4, H-7α, H-7B |
| | | β: 2.03 (1H, m) | C-5, C-7, C-10 | H-7β, 14-OH, H-19, H-20 |
| 7 | 24.1, CH₂ | α: 2.65 (1H, ddd, 16.4, 11.5, 7.8) | C-6, C-8, C-9, C-14 | H-5, H-6α, 14-OH |
| | | β: 2.82 (1H, dd, 16.4, 6.6) | C-5, C-6, C-8, C-9, C-14 | H-6α, H-6β |
| 8 | 120.7, C | — | | |
| 9 | 148.2, C | — | | |
| 10 | 37.5, C | — | | |
| 11 | 116.6, CH | 6.84 (1H, d, 8.2) | C-8, C-10, C-13 | H-1β, H-20 |
| 12 | 123.4, CH | 7.02 (1H, d, 8.2) | C-9, C-14, C-15 | H-16, H-17 |
| 13 | 130.0, C | — | | |
| 14 | 150.2, C | — | | |
| 14-OH | — | 4.64 (1H, br s) | H-8, H-13, H-14 | H-7α, H-7β, H-15 |
| 15 | 26.9, CH | 3.14 (1H, sep, 6.9) | C-12, C-13, C-14, C-16, C-17 | 14-OH |
| 16 | 22.7, CH₃ | 1.24 (3H, d, 6.9) | C-13, C-15, C-17 | H-12 |
| 17 | 22.6, CH₃ | 1.25 (3H, d, 6.9) | C-13, C-15, C-16 | H-12 |
| 18 | 65.8, CH₂ | A: 3.54 (1H, dd, 10.4, 6.8) | C-3, C-4, C-5 | H-2α, H-2β, H-3, H-19 |
| | | B: 3.60 (1H, dd, 10.4, 8.0) | C-3, C-4, C-5 | H-2β, H-3, H-4, H-19 |
| 19 | 9.7, CH₃ | 0.89 (3H, d, 7.6) | C-3, C-4, C-5 | H-2β, H-6β, H-18B, H-20 |
| 20 | 25.4, CH₃ | 1.16 (3H, s) | C-1, C-5, C-9, C-10 | H-1β, H-2α, H-6β, H-11, H-19 |

TABLE 15
¹H and ¹³C NMR data and 2D HMBC and ROESY correlations for F20P4

| Pos. | δ_{C}, type^{a, b} | δ_{H}, nH, multiplicity (J in Hz)^{a, c} | HMBC | ROESY |
|---|---|---|---|---|
| 1 | 33.3, CH₂ | α: 1.49 (1H, m) | C-10, C-20 | H1β, H-18 |
| | | β: 2.04 (1H, m) | C-3, C-5 | H1α, H-11, H-20 |
| 2 | 19.5, CH₂ | α: 1.66 (1H, m) | | H-18 |
| | | β: 2.00 (1H, m) | | H-19, H-20 |
| 3 | 42.9, CH | 1.72 (1H, m) | C-1, C-2, C-4, C-18, C-19 | |
| 4 | 34.7, CH | 1.91 (1H, m) | C-2, C-3, C-5, C-10, C-18, C-19 | H-6α, H-18 |
| 5 | 38.7, CH | 1.74 (1H, ddd, 12.8, 5.1, 2.2) | C-4, C-6, C-7, C-10, C-19 | |
| 6 | 23.4, CH₂ | α: 1.59 (1H, br dd, 12.8, 7.8) | C-4, C-5, C-7, C-8, C-10 | H-4, H-7α, H-7β |
| | | β: 1.98 (1H, m) | C-5, C-7, C-10 | H-7β, 14-OH, H-19, H-20 |
| 7 | 24.0, CH₂ | α: 2.62 (1H, ddd, 16.5, 11.4, 7.8) | C-6, C-8, C-9 | H-6α, 14-OH |
| | | β: 2.81 (1H, dd, 16.5, 6.7) | C-5, C-6, C-8, C-9, C-14 | H-6α, H-6β |
| 8 | 120.7, C | — | | |
| 9 | 148.2, C | — | | |
| 10 | 37.4, C | — | | |
| 11 | 116.4, CH | 6.82 (1H, d, 8.2) | C-8, C-10, C-13 | H-1β |
| 12 | 123.3, CH | 7.01 (1H, d, 8.2) | C-9, C-14, C-15 | H-16, H-17 |
| 13 | 130.0, C | — | | |
| 14 | 150.2, C | — | | |
| 14-OH | — | 4.63 (1H, br s) | H-8, H-13 | H-7α, H-7β, H-15 |
| 15 | 26.8, CH | 3.14 (1H, sep, 6.9) | C-12, C-13, C-14, C-16, C-17 | 14-OH |
| 16 | 22.7, CH₃ | 1.24 (3H, d, 6.9) | C-13, C-15, C-17 | H-12 |
| 17 | 22.6, CH₃ | 1.25 (3H, d, 6.9) | C-13, C-15, C-16 | H-12 |
| 18 | 64.7, CH₂ | 3.66 (2H, m) | C-2, C-4 | H-1α, H-2α, H-4 |
| 19 | 16.7, CH₃ | 1.09 (3H, d, 7.6) | C-3, C-4, C-5 | H-2β, H-6β, H-20 |
| 20 | 25.0, CH₃ | 1.21 (3H, s) | C-1, C-5, C-9, C-10 | H-1β, H-2β, H-6β, H-19 |

TABLE 16
¹H and ¹³C NMR data and 2D HMBC and ROESY correlations for F15P2

| Pos. | δ_{C}, type^{a, b} | δ_{H}, nH, multiplicity (J in Hz)^{a, c} | HMBC | ROESY |
|---|---|---|---|---|
| 1 | 32.7, CH₂ | α: 1.56 (1H, m) | C-2, C-5, C-10, C-20 | H-2α |
| | | β: 2.42 (1H, m) | C-2, C-3, C-5, C-10, C-20 | H-11, H-20 |
| 2 | 20.7, CH₂ | α: 2.53 (1H, m) | C-1, C-3, C-4, C-10 | H-1α, H-19 |
| | | β: 2.33 (1H, m) | | H-20 |
| 3 | 133.5, C | — | | |
| 4 | 156.1, C | — | | |
| 5 | 46.7, CH | 2.44 (1H, m) | | H-7α |
| 6 | 19.4, CH₂ | α: 2.38 (1H, m) | C-5, C-8, C-10 | H-7α |
| | | β: 1.76 (1H, tdd, 13.2, 10.9, 7.2) | C-5, C-7, C-10 | H-7β, H-20 |
| 7 | 23.4, CH₂ | α: 2.77 (1H, ddd, 17.0, 10.9, 8.2) | C-6, C-8, C-9, C-14 | H-5, H-6α, 14-OH |
| | | β: 2.93 (1H, dd, 17.0, 7.2) | C-5, C-6, C-8, C-9, C-14 | H-6β, 14-OH |
| 8 | 120.6, C | — | | |
| 9 | 145.1, C | — | | |
| 10 | 35.8, C | — | | |
| 11 | 116.4, CH | 6.94 (1H, d, 8.2) | C-8, C-10, C-13 | H-1β |
| 12 | 123.3, CH | 7.05 (1H, d, 8.2) | C-9, C-14, C-15 | H-16, H-17 |
| 13 | 130.6, C | — | | |
| 14 | 150.4, C | — | | |
| 14-OH | — | 4.68 (1H, s) | C-8, C-13, C-14 | H-7α, H-7β, H-15 |
| 15 | 26.9, CH | 3.12 (1H, sep, 6.9) | C-12, C-13, C-14, C-16, C-17 | 14-OH |
| 16 | 22.6, CH₃ | 1.26 (3H, d, 6.9) | C-13, C-15, C-17 | H-12 |
| 17 | 22.5, CH₃ | 1.27 (3H, d, 6.9) | C-13, C-15, C-16 | H-12 |
| 18 | 191.3, CH | 10.24 (1H, s) | C-2, C-3 | H-19 |
| 19 | 15.0, CH₃ | 2.22 (3H, ddd, 1.8, 1.6, 1) | C-3, C-4, C-5 | H-2α, H-18 |
| 20 | 22.7, CH₃ | 1.02 (3H, s) | C-1, C-5, C-9, C-10 | H-1β, H-2β, H-6β |

TABLE 17
¹H and ¹³C NMR data and 2D HMBC and ROESY correlations for F55P2

| Pos. | δ_{C}, type^{a, b} | δ_{H}, nH, multiplicity (J in Hz)^{a, c} | HMBC | ROESY |
|---|---|---|---|---|
| 1 | 28.5, CH₂ | α: 1.48 (1H, m) | | H-1β, H-5 |
| | | β: 2.67 (1H, ddd, 13.1, 5.2, 2.9) | | H-1α, H-2, H-11, H-20B |
| 2 | 25.7, CH₂ | 2.36 (2H, m) | | H-1β, H-18A, H-18B, H-20B |
| 3 | 130.6^{d}, C | — | | |
| 4 | 131.0^{d}, C | — | | |
| 5 | 43.6, CH | 2.41 (1H, br d, 14.2) | | H-1α |
| 6 | 20.1, CH₂ | α: 2.29 (1H, dddd, 13.6, 8.4, 3.3, ~1) | | H-7A, H-7B, H-19 |
| | | β: 1.70 (1H, tdd, 13.6, 10.4, 8.0) | | H-7B, H-20A, H-20B |
| 7 | 23.0, CH₂ | A: 2.78 (1H, m) | C-6, C-8 | H-6α, 14-OH |
| | | B: 2.88 (1H, m) | C-5, C-6, C-8, C-9 | H-6α, H-6β, 14-OH |
| 8 | 121.7, C | — | | |
| 9 | 140.8, C | — | | |
| 10 | 40.3, C | — | | |
| 11 | 117.2, CH | 6.98 (1H, d, 8.2) | C-8, C-10, C-13 | H-1β |
| 12 | 123.0, CH | 7.07 (1H, d, 8.2) | C-9, C-14, C-15 | H-16/17 |
| 13 | 131.5, C | — | | |
| 14 | 150.9, C | — | | |
| 14-OH | — | 4.72 (1H, br s) | C-8, C-13 | H-7A, H-7B, H-15 |
| 15 | 27.0, CH | 3.15 (1H, sep, 6.9) | C-12, C-13, C-14, C-16, C-17 | 14-OH |
| 16 | 22.6, CH₃ | 1.27 (3H, d, 6.9) | C-13, C-15, C-17 | H-12 |
| 17 | 22.6, CH₃ | 1.27 (3H, d, 6.9) | C-13, C-15, C-16 | H-12 |
| 18 | 63.2, CH₂ | A: 4.09 (1H, d, 11.6) | C-2, C-4 | H-2, H-19 |
| | | B: 4.28 (1H, d, 11.6) | C-2, C-4 | H-2, H-19 |
| 19 | 15.6, CH₃ | 1.79 (3H, q, 1.9) | C-3, C-4, C-5 | H-6α, H-18A, H-18B |
| 20 | 64.7, CH₂ | A: 3.56 (1H, dd, 10.8, 3.0) | | H-6β |
| | | B: 3.68 (1H, dd, 10.8, 7.7) | C-9 | H-1β, H-2, H-6β |
| 20-OH | — | 1.03 (1H, m) | | |

TABLE 18
¹H and ¹³C NMR data and 2D HMBC and ROESY correlations for F55P3

| Pos. | δ_{C}, type^{a, b} | δ_{H}, nH, multiplicity (J in Hz)^{a, c} | HMBC | ROESY |
|---|---|---|---|---|
| 1 | 38.4, CH₂ | α: 1.45 (1H, m) | C-2, C-20 | H1β, H-3 |
| | | β: 2.28 (1H, m) | C-3, C-5, C-10, C-20 | H1α, H-11, H-20 |
| 2 | 21.3, CH₂ | 1.46 (1H, m) | C-1, C-3, C-4, C-10 | H-18A, H-18B, H-19B, H-20 |
| 3 | 42.8, CH | 1.89 (1H, m) | | H1α, H-4, H-18B |
| 4 | 44.6, CH | 2.21 (1H, dt, 9.0, 4.6) | | H-3, H-5, H-6α, H-19A |
| 5 | 44.4, CH | 1.79 (1H, m) | C-6, C-7, C-19 | H-4, H-7α |
| 6 | 23.8, CH₂ | α: 1.80 (1H, m) | C-4, C-5, C-7, C-8, C-10 | H-4, H-7α, H-7β, H-19A |
| | | β: 1.98 (1H, tdd, 13.3, 11.6, 6.2) | C-5, C-7, C-10 | H-7β, 14-OH, H-19A, H-20 |
| 7 | 24.4, CH₂ | α: 2.66 (1H, ddd, 16.4, 11.6, | C-6, C-8 | H-5, H-6α, 14-OH |
| | | 7.4) | | |
| | | β: 2.85 (1H, dd, 16.4, 6.2) | C-5, C-6, C-8, C-9, C-14 | H-6α, H-6Bβ |
| 8 | 120.5, C | — | | |
| 9 | 146.9, C | — | | |
| 10 | 37.1, C | — | | |
| 11 | 116.9, CH | 6.83 (1H, d, 8.3) | C-8, C-10, C-13 | H-1β |
| 12 | 123.4, CH | 7.02 (1H, d, 8.3) | C-9, C-14, C-15 | H-16, H-17 |
| 13 | 130.1, C | — | | |
| 14 | 150.2, C | — | | |
| 14-OH | — | 4.64 (1H, s) | H-8, H-13, H-14 | H-7α, H-7β, H-15 |
| 15 | 26.8, CH | 3.13 (1H, sep, 6.9) | C-12, C-13, C-14, C-16, C-17 | 14-OH |
| 16 | 22.7, CH₃ | 1.24 (3H, d, 6.9) | C-13, C-15, C-17 | H-12 |
| 17 | 22.5, CH₃ | 1.25 (3H, d, 6.9) | C-13, C-15, C-16 | H-12 |
| 18 | 65.4, CH₂ | A: 3.63 (1H, m) | | H-2, H-19B |
| | | B: 3.65 (1H, m) | C-3, C-4 | H-2, H-3, H-19B |
| 19 | 59.5, CH₂ | A: 3.71 (1H, d, 10.4) | C-3, C-4 | H-4, H-6α, H-6β, H-20 |
| | | B: 3.88 (1H, dd, 10.4, 9.2) | C-3, C-4 | H-2, H-18A/B, H-20 |
| 20 | 24.6, CH₃ | 0.98 (3H, s) | C-1, C-5, C-9, C-10 | H-1β, H-2, H-6β, H-19A, H-19B |

TABLE 19
¹H and ¹³C NMR data and 2D HMBC and ROESY correlations for F15P4

| Pos. | δ_{C}, type^{a, b} | δ_{H}, nH, multiplicity (J in Hz)^{a, c} | HMBC | ROESY |
|---|---|---|---|---|
| 1 | 37.0, CH₂ | α: 1.16 (1H, td, 13.2, 3.7) | C-2, C-5, C-10, C-20 | H-3 |
| | | β: 1.79 (1H, dt, 13.2, 3.5) | C-3, C-5 | H-20 |
| 2 | 27.9, CH₂ | A: 1.60 (1H, m) | C-1, C-3 | H-19 |
| | | B: 1.71 (1H, m) | C-3 | |
| 3 | 78.8, CH | 3.25 (1H, dd, 11.8, 4.3) | C-4, C-18, C-19 | H-1α, H-5, H-18 |
| 4 | 39.1, C | — | | |
| 5 | 54.6, CH | 1.08 (1H, dd, 12.5, 2.7) | C-4, C-6, C-7, C-9, C-10, C-18, C-19, | H-3, H-7α, H-9, H-18 |
| | | | C-20 | |
| 6 | 24.0, CH₂ | α: 1.74 (1H, dddd, 13.0, 5.0, 2.7, 2.5) | C-5, C-7, C-8, C-10 | H-7β, H-18 |
| | | β: 1.39 (1H, tdd, 13.0, 12.5, 4.2) | C-5, C-7, C-10 | H-7β, H-19 |
| 7 | 38.1, CH₂ | α: 1.96 (1H, ddd, 13.0, 12.8, 5.0) | C-6, C-8, C-17 | H-5 |
| | | β: 2.40 (1H, ddd, 12.8, 4.2, 2.5) | C-5, C-6, C-8, C-9, C-17 | H-6α, H-6β, H-17B |
| 8 | 147.8, C | — | | |
| 9 | 55.9, CH | 1.54 (1H, m) | C-7, C-8, C-10, C-11, C-12, C-17, C-20 | H-5, H-12B, H-14, H-17A, H-20 |
| 10 | 39.2, C | — | | |
| 11 | 21.8, CH₂ | A: 1.47 (1H, m) | C-8, C-9, C-12, C-13 | H-12B, H-14, H-17A, H-20 |
| | | B: 1.59 (1H, m) | C-8, C-9, C-12 | |
| 12 | 38.3, CH₂ | A: 1.83 (1H, ddd, 14.0, 9.4, 6.6) | C-9, C-11, C-13, C-14, C-15 | H-14 |
| | | B: 2.17 (1H, ddd, 14.0, 10.0, 3.8) | C-11, C-13, C-14 | H-9, H-11A, H-14, H-15, H-17A |
| 13 | 142.7, C | — | | |
| 14 | 118.0, CH | 5.31 (1H, t sext, 7.1, 1.2) | C-12, C-15, C-16 | H-9, H-11A, H-12A, H-12B |
| 15 | 16.5, CH₃ | 1.69 (3H, br s) | C-12, C-13, C-14 | H-12B, H-16 |
| 16 | 61.4 CH₂ | 4.58 (2H, d, 7.1) | C-13, C-14, C-1 | H-15 |
| 17 | 106.0, CH₂ | A: 4.53 (1H, q, ~1) | C-7, C-8, C-9 | H-9, H-11A, H-12A, H-12B, H-17B |
| | | B: 4.85 (1H, q, ~1.4) | C-7, C-9 | H-7β, H-17A |
| 18 | 28.3, CH₃ | 1.00 (3H, s) | C-3, C-4, C-5, C-19 | H-3, H-5, H-6α |
| 19 | 15.3, CH₃ | 0.78 (3H, s) | C-3, C-4, C-5, C-18 | H-2A, H-6β |
| 20 | 14.5, CH₃ | 0.69 (3H, s) | C-1, C-5, C-9, C-10 | H-9, H-11A |
| 1′ | 171.0, C | — | | |
| 2′ | 21.1, CH₃ | 2.06 (3H, s) | C-1′ | |

TABLE 20
¹H and ¹³C NMR data and 2D HMBC and ROESY correlations for F20P5

| Pos. | δ_{C}, type^{a, b} | δ_{H}, nH, multiplicity (J in Hz)^{a, c} | HMBC | ROESY |
|---|---|---|---|---|
| 1 | 59.4, CH₂ | 4.16 (2H, d, 6.9) | C-2, C-3 | H-3-Me |
| 2 | 123.4, CH | 5.42 (1H, t sext, 6.9, 1.3) | C-3-Me, C-4 | H-4 |
| 3 | 139.6, C | — | | |
| 3-Me | 16.3, CH₃ | 1.68 (3H, br s) | C-2, C-3, C-4 | H-1 |
| 4 | 39.5, CH₂ | 2.04 (2H, m) | C-2, C-3, C-3-Me, C-5 | H-2 |
| 5 | 26.3, CH₂ | 2.11 (2H, m) | C-4, C-6, C-7 | |
| 6 | 123.9, CH | 5.11 (1H, t sext, 6.9, 1.2) | C-5, C-7-Me, C-8 | |
| 7 | 135.1, C | — | | |
| 7-Me | 15.9, CH₃ | 1.60 (3H, br s) | C-6, C-7, C-8 | |
| 8 | 39.6, CH₂ | 1.99 (2H, m) | C-6, C-7, C-7-Me, C-9, C-10 | |
| 9 | 26.5, CH₂ | 2.08 (2H, m) | C-7, C-8, C-10, C-11 | |
| 10 | 124.8, CH | 5.16 (1H, t sext, 6.9, 1.2) | C-9, C-11-Me, C-12 | H-12B, H-13 |
| 11 | 134.0, C | — | | |
| 11-Me | 16.0, CH₃ | 1.62 (3H, br s) | C-10, C-11, C-12 | |
| 12 | 36.3, CH₂ | A: 2.08 (1H, m) | C-10, C-11, C-11-Me, C-13, C-14 | H-14 |
| | | B: 2.16 (1H, m) | C-10, C-11, C-11-Me, C-13, C-14 | H-10, H-14 |
| 13 | 27.4, CH₂ | 1.63 (2H, m) | C-11, C-12, C-14, C-15 | H-10 |
| 14 | 64.1, CH | 2.70 (1H, t, 6.3) | C-12, C-13, C-15, C-15-Me′ | H-12A, H-12B, |
| | | | | H-15-Me, H-15-Me′ |
| 15 | 58.2, C | — | | |
| 15-Me | 18.7, CH₃ | 1.26 (3H, s) | C-14, C-15, C-15-Me' | H-14 |
| 15-Me′ | 24.8, CH₃ | 1.30 (3H, s) | C-14, C-15, C-15-Me | H-14 |

TABLE 21
¹H and ¹³C NMR data and 2D HMBC and ROESY correlations for F60P1

| Pos. | δ_{C}, type^{a, b} | δ_{H}, nH, multiplicity (J in Hz)^{a, c} | HMBC | ROESY |
|---|---|---|---|---|
| 1 | 59.3, CH2 | 4.15 (2H, br d, 6.9) | C-2, C-3 | H-3-Me |
| 2 | 123.7, CH | 5.42 (1H, t sext, 6.9, 1.2) | C-3-Me, C-4 | H-4 |
| 3 | 139.3, C | — | | |
| 3-Me | 16.2, CH3 | 1.68 (3H, br s) | C-2, C-3, C-4 | H-1, H-4 |
| 4 | 39.3, CH2 | 2.07 (2H, m) | | H-2, H-3-Me |
| 5 | 25.9, CH2 | 2.14 (2H, m) | C-4, C-6, C-7 | |
| 6 | 124.3, CH | 5.13 (1H, t sext, 6.8, 1.2) | C-4, C-5, C-7-Me, C-8 | |
| 7 | 136.2, C | — | | |
| 7-Me | 16.0, CH3 | 1.64 (3H, br s) | C-6, C-7, C-8 | H-8 |
| 8 | 42.5, CH2 | 2.10 (2H, m) | C-6, C-7, C-7-Me, C-9, C-10 | H-7-Me, H-10, H-15-αMe |
| 9 | 24.2, CH2 | A: 1.48 (1H, m) | C-8, C-10, C-15 | |
| | | B: 1.55 (1H, m) | C-8, C-10, C-11 | H-11-Me |
| 10 | 55.3, CH | 1.10 (1H, t, 4.3) | C-8, C-9, C-11, C-11-Me, C-15 | H-8, H-14 |
| 11 | 73.4, C | — | | |
| 11-Me | 23.0, CH3 | 1.16 (3H, s) | C-10, C-11, C-12 | H-9B, H-12B, H-13A, H-15-βMe |
| 12 | 40.9, CH2 | A: 1.45 (1H, m) | C-13 | |
| | | B: 1.78 (1H, m) | C-10, C-11, C-14 | H-11-Me |
| 13 | 28.9, CH2 | A: 1.49 (1H, m) | C-14 | H-11-Me |
| | | B: 1.75 (1H, m) | C-10, C-11, C-14 | H-14 |
| 14 | 78.3, CH | 3.31 (1H, dd, 10.6, 2.8) | | H-10, H-13B, H-15-αMe |
| 15 | 40.4, C | — | | |
| 15-αMe | 28.0, CH3 | 1.04 (3H, s) | C-10, C-14, C-15, C-15-βMe | H-8, H-14 |
| 15-βMe | 14.8, CH3 | 0.80 (3H, s) | C-10, C-14, C-15, C-15-αMe | H-11-Me |

^{a1}H NMR (600.13) and ¹³C NMR (150.90 MHz) data obtained with samples in CDCl₃.
^{b}Assignments based on HSQC and HMBC experiments.
^{c}Multiplicities reported as apparent splittings: s = singlet, d = doublet, t = triplet, sext = sextet, m = multiplet (incl. overlapping resonances), br = broad.
α denotes Me pointing into the plane and β denotes Me pointing out of the plane. A denotes the lowest chemical shift value and B denotes the highest chemical shift value

### REFERENCES

1. J. Andersen-Ranberg et al., Expanding the Landscape of Diterpene Structural Diversity through Stereochemically Controlled Combinatorial Biosynthesis. Angewandte Chemie International Edition, n/a (2016).
2. I. Pateraki et al., Total biosynthesis of the cyclic AMP booster forskolin from Coleus forskohlii. Elife 6, e23001 (2017).
3. I. Pateraki et al., Manoyl Oxide (13R), the Biosynthetic Precursor of Forskolin, Is Synthesized in Specialized Root Cork Cells in Coleus forskohlii. Plant Physiology 164, 1222-1236 (2014).
4. N. L. Hansen et al., The terpene synthase gene family in Tripterygium wilfordii harbors a labdane-type diterpene synthase among the monoterpene synthase TPS-b subfamily. The Plant Journal 89, 429-441 (2017).
5. H. H. Nour-Eldin, B. G. Hansen, M. H. H. Nørholm, J. K. Jensen, B. A. Halkier, Advancing uracil-excision based cloning towards an ideal technique for cloning PCR fragments. Nucleic Acids Research 34, e122 (2006).
6. N. B. Jensen et al., EasyClone: method for iterative chromosomal integration of multiple genes in Saccharomyces cerevisiae. FEMS Yeast Research 14, 238-248 (2014).
7. M. H. H. Nørholm, A mutant Pfu DNA polymerase designed for advanced uracilexcision DNA engineering. BMC Biotechnology 10, 21 (2010).
8. R. D. Gietz, R. H. Schiestl, High-efficiency yeast transformation using the LiAc/SS carrier DNA/PEG method. Nature Protocols 2, 31-34 (2007).
9. Hansen, N. L., et al., Integrating pathway elucidation with yeast engineering to produce polpunonic acid the precursor of the anti-oDesity agent celastrol. MicroD Cell Fact, 2020. 19 (1): p. 15.
10. Voinnet O, Rivas S, Mestre P, Baulcombe D. An enhanced transient expression system in plants based on suppression of gene silencing by the p19 protein of tomato bushy stunt virus [retracted in: Plant J. 2015 November; 84 (4): 846]. Plant J. 2003; 33 (5): 949-956. doi: 10.1046/j. 1365-313×.2003.01676.x

### Items

1. A recombinant host cell, capable of producing oxygenated diterpenoid compound, wherein the host cell is capable of producing miltiradiene and/or dehydroabietadiene and has been transformed with a first gene encoding an enzyme having cytochrome P450 activity and which enzyme is capable of converting miltiradiene and/or dehydroabietadiene into 14-OH-dehydroabietadiene.
2. The recombinant host cell of item 1, wherein the first gene encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 1 (TwCYP82D274V1) or the mature polypeptide thereof.
3. The recombinant host cell of item 1 or 2, wherein the recombinant host cell further comprises:
   a second gene encoding a second enzyme having cytochrome P450 activity and a third gene encoding a third enzyme having cytochrome P450 activity, wherein:
      the second gene encoding an enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 4 (TwCYP71BE86) or the mature polypeptide thereof;
      the third gene encoding an enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 3 (TwCYP71BE85) or the mature polypeptide thereof.
4. The recombinant host cell of item 3, wherein the host cell further comprises a gene encoding a polypeptide having cytochrome B5 activity and comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 8 (TwB5 #1) or the mature polypeptide thereof.
5. The recombinant host cell of item 3 or 4, wherein the host cell further comprises a fourth gene encoding a fourth enzyme having cytochrome P450 activity; wherein:
   the fourth gene encoding a fourth enzyme having cytochrome P450 activity encodes a polypeptide comprising an amino acid sequence having at least 80% sequence identity, preferably at least 85% sequence identity, preferably at least 90% sequence identity, preferably at least 95% sequence identity more preferred at least 98% sequence identity to SEQ ID NO: 5 (TwCYP82D213) or the mature polypeptide thereof.
6. The recombinant host cell of item 5, wherein the host cell further comprises a fifth gene encoding a fifth enzyme having cytochrome P450 activity and/or a sixth gene encoding a sixth enzyme having cytochrome P450 activity, wherein:
   the fifth gene encoding a fifth enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 6 (TwCYP82D217) or the mature polypeptide thereof; and the sixth gene encoding a sixth enzyme having cytochrome P450 activity encodes a polypeptide comprising an amino acid sequence having at least 80% sequence identity, preferably at least 85% sequence identity, preferably at least 90% sequence identity, preferably at least 95% sequence identity, more preferred at least 98% sequence identity to SEQ ID NO: 7 (TwCYP82D275) or the mature polypeptide thereof.
7. The recombinant host cell according to any of the preceding items, wherein the host cell capable of producing miltiradiene and/or dehydroabietadiene is a recombinant cell that has been transformed with one or more gene(s) encoding:
   a. a geranylgeranyl diphosphate synthase;
   b. a diterpene synthase capable of converting GGPP into miltiradiene;
   c. a combination of two or more diterpene synthases that in combination is capable of converting GGPP into miltiradiene; or
   d. a copalyl diphosphate synthase and a miltiradiene synthase.
8. The recombinant host cell of item 7, wherein the geranylgeranyl diphosphate synthase is a polypeptide comprising the amino acid sequence of SEQ ID NO: 73 or SEQ ID NO: 81.
9. The recombinant host cell of item 7, wherein the combination of two or more diterpene synthases, that is capable of converting GGPP into miltiradiene, is the combination of a polypeptide comprising the amino acid sequence of SEQ ID NO: 67 and a polypeptide comprising the amino acid sequence of SEQ ID NO: 68; or is the combination of a polypeptide comprising the amino acid sequence of SEQ ID NO: 69 and a polypeptide comprising the amino acid sequence of SEQ ID NO: 70.
10. The recombinant host cell of item 7, wherein the combination of a copalyl diphosphate synthase and a miltiradiene synthase is a combination of a polypeptide comprising the amino acid sequence of SEQ ID NO: 71 and a polypeptide comprising the polypeptide of SEQ ID NO: 72.
11. The recombinant host cell according to any of the previous items wherein the host cell is selected among prokaryotic and eukaryotic cells.
12. The recombinant host cell according to item 11, being a prokaryotic cell selected among Escherichia, Bacillus, Lactobacillus and Corynebacterium species.
13. The recombinant host cell of item 11, being a eukaryotic cell selected among Saccharomyces, Scizosaccharomyces, Klyveromyces, Pichia, Candida and Yarrowia species.
14. The recombinant host cell of item 11, where the cell is a S. cerevisiae cell.
15. Use of a recombinant host cell according to any of the preceding items for the production of an oxygenated diterpenoid compound.
16. The use of item 15, wherein the oxygenated diterpenoid compound is selected among 14-OH-dehydroabietadiene, triptophenolide and triptonide.
17. The use of item 16, wherein the oxygenated diterpenoid compound is triptonide, which triptonide is further converted into triptolide.
18. The use according to one of the items 15-17, wherein the oxygenated diterpenoid compound is recovered using one or more separation and/or chromatographic steps.
19. A polypeptide having cytochrome P450 enzyme activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, or even 100% sequence identity to one of the sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or the mature polypeptide thereof.
20. A polynucleotide encoding the polypeptide of item 19.
21. A plasmid, expression vector, expression construct or recombinant host cell comprising a polynucleotide of item 20.
22. The compound 14-OH-dehydroabietadiene.

1. A recombinant host cell capable of producing oxygenated diterpenoid compounds, wherein the host cell
i. is capable of producing miltiradiene and/or dehydroabietadiene; and
ii. comprises a first heterologous nucleic acid encoding a first enzyme having cytochrome P450 activity, wherein the first enzyme having cytochrome P450 activity is the cytochrome P450 enzyme TwCYP82D274 as set forth in SEQ ID NO: 1, SEQ ID NO: 2, SEQ ID NO: 74 or SEQ ID NO: 75, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof, whereby the host cell is capable of converting miltiradiene and/or dehydroabietadiene into 14-hydroxydehydroabietadiene.

2. The recombinant host cell according to claim 1, wherein the recombinant host cell further comprises a second heterologous nucleic acid encoding a second enzyme having cytochrome P450 activity, wherein the second enzyme having cytochrome P450 activity is the cytochrome P450 enzyme TwCYP71BE86 as set forth in SEQ ID NO: 4, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof.

3. The recombinant host cell according to claim 2, wherein the recombinant host cell comprises and expresses said first heterologous nucleic acid and said second heterologous nucleic acid, whereby the cell is capable of producing 14-hydroxydehydroabietadiene, 3,14-dihydroxydehydroabietadiene, 3,14-dihydroxyabeodiene and 14-hydroxy-18-aldo-abeodiene.

4. The recombinant host cell according to claim 1, wherein the recombinant host cell further comprises a third heterologous nucleic acid encoding a third enzyme having cytochrome P450 activity, wherein the third enzyme having cytochrome P450 activity is the cytochrome P450 enzyme TwCYP71BE85 as set forth in SEQ ID NO: 3, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof.

5. The recombinant host cell according to claim 4, wherein the recombinant host cell comprises and expresses said first heterologous nucleic acid and said third heterologous nucleic acid, whereby the cell is capable of producing 14-hydroxydehydroabietadiene.

6. The recombinant host cell according to claim 4, wherein the recombinant host cell comprises and expresses said first heterologous nucleic acid, said second heterologous nucleic acid and said third heterologous nucleic acid, whereby the cell is capable of producing 14-hydroxydehydroabietadiene, 3,14-dihydroxydehydroabietadiene, 3,14-dihydroxyabeodiene, 14-hydroxy-18-aldo-abeodiene and triptophenolide.

7. The recombinant host cell according to claim 1, wherein the recombinant host cell further comprises a fourth heterologous nucleic acid encoding a fourth enzyme having cytochrome P450 activity, wherein the fourth enzyme having cytochrome P450 activity is the cytochrome P450 enzyme TwCYP82D213 as set forth in SEQ ID NO: 5 or SEQ ID NO: 76, or a functional homologue thereof having at least 80% sequence identity, preferably at least 85% sequence identity, preferably at least 90% sequence identity, preferably at least 95% sequence identity more preferred at least 98% sequence identity thereto, or the mature polypeptide thereof.

8. The recombinant host cell according to claim 7, wherein the recombinant host cell comprises and expresses said first heterologous nucleic acid, said second heterologous nucleic acid, said third heterologous nucleic acid and said fourth heterologous nucleic acid, whereby the cell is capable of producing 14-hydroxydehydroabietadiene, 3,14-dihydroxydehydroabietadiene, 3,14-dihydroxyabeodiene, 14-hydroxy-18-aldo-abeodiene, triptophenolide and triptonide.

9. The recombinant host cell according to claim 1, wherein the host cell further comprises a fifth heterologous nucleic acid encoding a fifth enzyme having cytochrome P450 activity, wherein the fifth enzyme having cytochrome P450 activity is the cytochrome P450 enzyme TwCYP82D217 as set forth in SEQ ID NO: 6, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof.

10. The recombinant host cell according to claim 1, wherein the host cell further comprises a sixth heterologous nucleic acid encoding a sixth enzyme having cytochrome P450 activity, wherein the sixth enzyme having cytochrome P450 activity is the cytochrome P450 enzyme TwCYP82D275 as set forth in SEQ ID NO: 7, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof.

11. The recombinant host cell according to claim 1, wherein the host cell further comprises a seventh heterologous nucleic acid encoding an enzyme having cytochrome B5 activity, wherein the enzyme having cytochrome B5 activity is the cytochrome B5 TwB5 #1 as set forth in SEQ ID NO: 8, or a functional homologue thereof having at least 80% sequence identity, preferably at least 85% sequence identity, preferably at least 90% sequence identity, preferably at least 95% sequence identity, more preferred at least 98% sequence identity thereto, or the mature polypeptide thereof.

12. The recombinant host cell according to claim 1, wherein the recombinant host cell expresses one or more of:
i. a geranylgeranyl diphosphate synthase;
ii. a diterpene synthase capable of converting geranyl-geranyl-diphosphate (GGPP) into miltiradiene;
iii. a combination of two or more diterpene synthases that in combination are capable of converting GGPP into miltiradiene; or
iv. a copalyl diphosphate synthase and a miltiradiene synthase
whereby the cell is capable of producing miltiradiene and/or dehydroabietadiene.

13. The recombinant host cell of claim 12, wherein the geranylgeranyl diphosphate synthase is a polypeptide comprising the amino acid sequence of SEQ ID NO: 73 or SEQ ID NO: 81, or a functional homologue thereof having at least 80% sequence identity, preferably at least 85% sequence identity, preferably at least 90% sequence identity, preferably at least 95% sequence identity, more preferred at least 98% sequence identity thereto.

14. The recombinant host cell according to claim 12, wherein the combination of two or more diterpene synthases, that is capable of converting GGPP into miltiradiene, is the combination of CfTPS1 as set forth in SEQ ID NO: 67 and CfTPS3 as set forth in SEQ ID NO: 68, or CftTPS1 as set forth in SEQ ID NO: 77 and CftTPS3 as set forth in SEQ ID NO: 78, or a combination of the respective functional homologues thereof having at least 80% sequence identity, preferably at least 85% sequence identity, preferably at least 90% sequence identity, preferably at least 95% sequence identity, more preferred at least 98% sequence identity thereto; or is the combination of TwTPS9 as set forth in SEQ ID NO: 69 and TwTPS27 as set forth in SEQ ID NO: 70, or a combination of the respective functional homologues thereof having at least 80% sequence identity, preferably at least 85% sequence identity, preferably at least 90% sequence identity, preferably at least 95% sequence identity, more preferred at least 98% sequence identity thereto.

15. The recombinant host cell according to claim 12, wherein the combination of a copalyl diphosphate synthase and a miltiradiene synthase is the combination of SmCPS as set forth in SEQ ID NO: 71 and SmKSL as set forth in SEQ ID NO: 72, or a combination of the respective functional homologues thereof having at least 80% sequence identity, preferably at least 85% sequence identity, preferably at least 90% sequence identity, preferably at least 95% sequence identity, more preferred at least 98% sequence identity thereto.

16. The recombinant host cell according to claim 1, wherein the recombinant host cell is a prokaryotic or a eukaryotic cell.

17. The recombinant host cell according to claim 1, wherein the recombinant host cell is a eukaryotic cell of a species selected from the group consisting of Saccharomyces, Schizosaccharomyces, Klyveromyces, Pichia, Candida and Yarrowia.

18. The recombinant host cell according to claim 1, wherein the recombinant host cell is a S. cerevisiae cell.

19. The recombinant host cell according to claim 1, wherein the recombinant host cell is a prokaryotic cell of a species selected from the list consisting of Escherichia, Bacillus, Lactobacillus and Corynebacterium.

20. The recombinant host cell according to claim 1, wherein the recombinant host cell is a plant cell or comprised in a plant, wherein the plant may be Nicotiana tabacum, and/or the host cell is a cell from another multicellular host.

21. A method for production of an oxygenated diterpenoid compound, such as triptonide, said method comprising the steps of
i. providing a recombinant host cell according to claim 1; and
ii. culturing said recombinant host cell under conditions suitable for production of said oxygenated diterpenoid compound.

22. The method according to claim 21, wherein the oxygenated diterpenoid compound is selected from the list consisting of 14-OH-dehydroabietadiene, triptophenolide and triptonide.

23. The method according to claim 21, wherein the oxygenated diterpenoid compound is triptonide.

24. The method according to claim 22, further comprising a step of recovering and, optionally, purifying the triptonide.

25. A method of producing triptolide said method comprising
i. producing triptonide according to the method of claim 22, and
ii. converting the triptonide into triptolide and,
iii. optionally, recovering and/or purifying the triptolide.

26. (canceled)

27. (canceled)

28. (canceled)

29. (canceled)

30. A polypeptide having cytochrome P450 enzyme activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, or even 100% sequence identity to one of the sequences SEQ ID NO: 1, SEQ ID NO: 2, SEQ ID NO: 3, SEQ ID NO: 4, SEQ ID NO: 5, SEQ ID NO: 6, SEQ ID NO: 7, or the mature polypeptide thereof or a polypeptide having cytochrome B5 enzyme activity and comprising an amino acid sequence having at least 80% sequence identity, preferably at least 85% sequence identity, preferably at least 90% sequence identity, preferably at least 95% sequence identity, preferably at least 96% sequence identity, preferably at least 97% sequence identity, preferably at least 98% sequence identity, or even 100% sequence identity to SEQ ID NO:8.

31. A polynucleotide encoding the polypeptide of claim 30.

32. A plasmid, expression vector, expression construct or recombinant host cell comprising the polynucleotide of claim 31.

33. The compound 14-OH-dehydroabietadiene.

34. A compound selected from the group consisting of the following formulas (1) to (17):

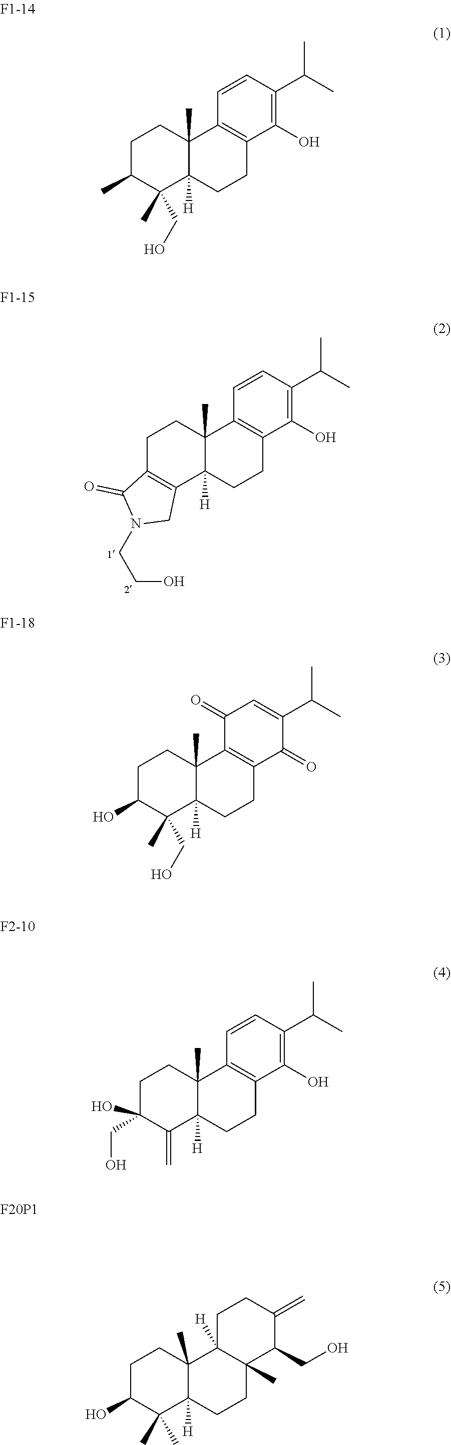

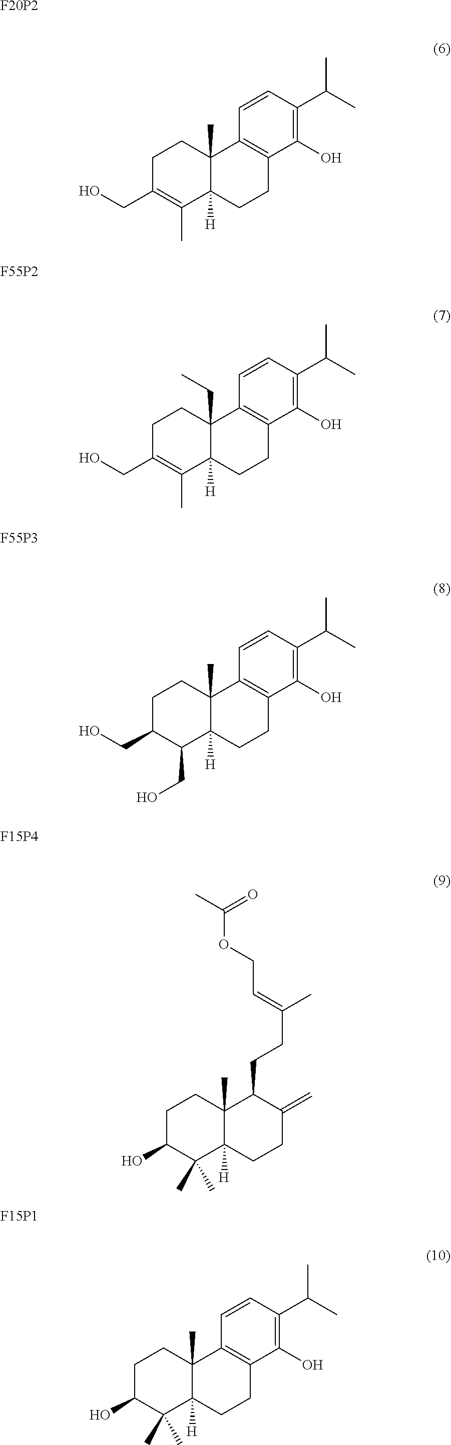

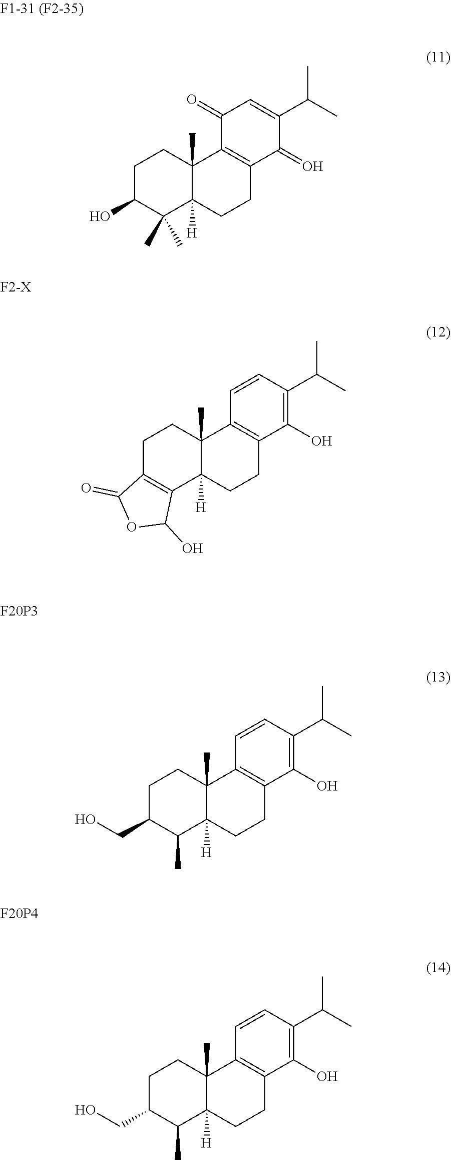

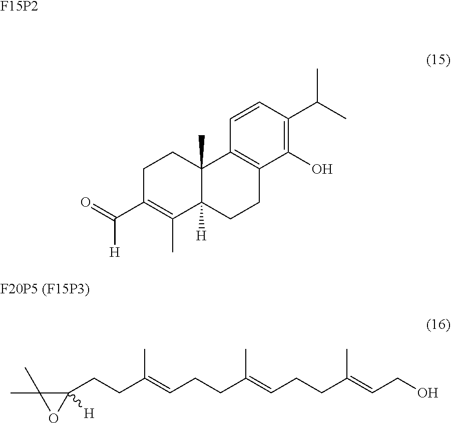

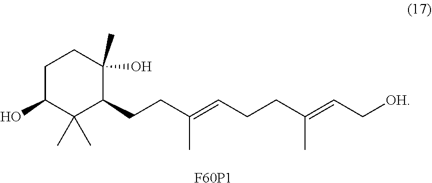

35. The compound according to claim 34, wherein said compound is the compound according to formula (6) (F20P2).

36. The compound according to claim 34, wherein said compound is the compound according to formula (10) (F15P1).

37. The compound according to claim 34, wherein said compound is the compound according to formula (15) (F15P2).
